(19) United States
(12) Patent Application Publication
(10) Pub. No.: US 2025/0027116 A1
(43) Pub. Date: Jan. 23, 2025

(54) METHODS FOR LARGE-SIZE CHROMOSOMAL TRANSFER AND MODIFIED CHROMOSOMES AND ORGANISIMS USING SAME

(71) Applicant: IMMUNOCAN BIOTECH CO. LTD., Shanghai (CN)

(72) Inventors: Jiwei ZHANG, Shanghai (CN); Yu WEI, Shanghai (CN)

(73) Assignee: IMMUNOCAN BIOTECH CO. LTD., Shanghai (CN)

(21) Appl. No.: 18/694,660

(22) PCT Filed: Sep. 23, 2022
(86) PCT No.: PCT/CN2022/120692

§ 371 Date: Mar. 22, 2024

(30) Foreign Application Priority Data
Sep. 24, 2021 (WO) PCT/CN2021/120126

(51) Int. Cl. C12N 15/90; A01K 67/0278; C07K 16/00; C12N 5/073; C12N 5/26; C12N 9/22; C12N 15/11

(52) U.S. Cl. CPC C12N 15/907; A01K 67/0278; C07K 16/00; C12N 5/0603; C12N 5/166; C12N 9/22; C12N 15/11; A01K 2207/15; A01K 2217/072; A01K 2227/105; C12N 2310/20; C12N 2510/00; C12N 2800/80

(57) ABSTRACT

Methods of transferring large sequence fragments between chromosomes and generating chromosomal rearrangements using double strand break repair pathways and homology directed repair. Further relates to chromosomes produced by these methods, and cells and transgenic animals comprising these chromosomes.

### INCORPORATION BY REFERENCE OF SEQUENCE LISTING

This application contains a Sequence Listing which has been submitted in ASCII format via EFS-WEB and is hereby incorporated by reference in its entirety.

### BACKGROUND

Manipulation of large fragments of genes or chromosomes is a powerful tool for basic and translational research as well as development of therapies. Human genes range in size from a few hundred bases, to at least 2,300 kilobases (KB), and human chromosomes range in size from 38 Megabasepairs (MB) to nearly 250 MB. Thus, the effective study of large genes, regions spanning multiple genes, and parts of chromosomes requires manipulating large sequence fragments. However, large fragment manipulation remains one of the most significant challenges in the gene editing field. The disclosure provides methods for manipulating large sequences.

### SUMMARY

The disclosure provides methods of generating an engineered chromosome, comprising: (a) providing a cell comprising a target chromosome comprising a target sequence and a template chromosome comprising a template sequence; (b) contacting the cell with (i) a first nucleic acid molecule comprising from 5′ to 3′, a 5′ homology arm comprising a nucleotide sequence upstream of the 5′ end of the target sequence, at least a first marker, and a 3′ homology arm comprising a nucleotide sequence upstream of the 5′ end of the template sequence; and (ii) a second nucleic acid molecule comprising from 5′ to 3′, a 5′ homology arm comprising a nucleotide sequence downstream of the 3′ end of the template sequence, at least a second marker, and a 3′ homology arm comprising a nucleotide sequence downstream of the 3′ end of the target sequence; (c) generating a double strand break at or on both sides of the target sequence, and at the 5′ and 3′ ends of the template sequence, whereby the template sequence and the first and second markers are inserted into the target chromosome; and (d) selecting a cell or cells expressing the first and second markers.

In some embodiments, the first marker is located at the 5′ end of the template sequence and the second marker is located at the 3′ end of the template sequence following insertion of the template sequence.

In some embodiments, the 5′ and 3′ homology arms of the first and second nucleic acid molecules are between about 20 and 2,000 base pairs (bp), between about 50 and 1,500 bp, between about 100 and 1,400 bp, between about 150 and 1,300 bp, between about 200 and 1,200 bp, between about 300 and 1,100 bp, between about 400 and 1,000 bp, or between about 500 and 900 bp, or between about 600 bp and 800 bp in length. In some embodiments, the 5′ and 3′ homology arms of the first and second nucleic acid molecules are between about 400 and 1,500 bp in length, between about 500 and 1,300 bp in length, or between about 600 and 1,000 bp in length. In some embodiments, the 5′ and 3′ homology arms of the first and second nucleic acid molecules are between about 600 and 1,000 bp in length.

In some embodiments, the template sequence is at least 25 kilobasepairs (KB), at least 50 KB, at least 100 KB, at least 200 KB, at least 400 KB, at least 500 KB, at least 600 KB, at least 700 KB, at least 800 KB, at least 900 KB, at least 1 megabasepairs (MB), at least 2 MB, at least 3 MB, at least 4 MB, at least 5 MB, at least 6 MB, at least 7 MB, at least 8 MB, at least 9 MB, at least 10 MB, at least 15 MB, at least 20 MB, at least 25 MB, at least 30 MB, at least 40 MB, at least 50 MB, at least 60 MB, at least 70 MB, at least 80 MB, at least 90 MB, at least 100 MB, at least 120 MB, at least 140 MB, at least 160 MB, at least 180 MB, at least 200 MB, at least 220 MB, or at least 250 MB in length. In some embodiments, the template sequence is between 50 KB and 250 MB, 50 KB and 100 MB, 50 KB and 50 MB, 50 KB and 20 MB, 50 KB and 10 MB, 50 KB and 5 MB, 50 KB and 3 MB, 50 KB and 2 MB, 50 KB and 1 MB, 100 KB and 200 MB, 100 KB and 100 MB, 100 KB and 50 MB, 100 KB and 20 MB, 100 KB and 10 MB, 100 KB and 5 MB, 100 KB and 3 MB, 100 KB and 2 MB, 100 KB and 1 MB, 100 KB and 500 KB, 200 KB and 100 MB, 200 KB and 50 MB, 200 KB and 20 MB, 200 KB and 10 MB, 200 KB and 5 MB, 200 KB and 3 MB, 200 KB and 2 MB, 200 KB and 1 MB, 200 KB and 500 KB, 500 KB and 100 MB, 500 KB and 50 MB, 500 KB and 20 MB, 500 KB and 10 MB, 500 KB and 5 MB, 500 KB and 3 MB, 500 KB and 2 MB, 500 KB and 1 MB, 1 MB and 100 MB, 1 MB and 50 MB, 1 MB and 20 MB, 1 MB and 10 MB, 1 MB and 5 MB, 1 MB and 3 MB, 1 MB and 2 MB, 3 MB and 100 MB, 3 MB and 50 MB, 3 MB and 20 MB, 3 MB and 10 MB, 3 MB and 5 MB, 5 MB and 100 MB, 5 MB and 501 MB, 5 MB and 20 MB, 5 MB and 10 MB, 10 MB and 100 MB, 10 MB and 50 MB, or 10 MB and 20 MB, in length. In some embodiments, the template sequence is between 200 KB and 50 MB, between 1 MB and 20 MB, between 1 MB and 10 MB, between 1 MB and 5 MB, between 1 MB and 3 MB, between 3 MB and 20 MB, between 3 MB and 10 MB, between 3 MB and 7 MB, or between 3 MB and 5 MB in length.

In some embodiments, generating the double strand breaks at (c) comprises using a CRISPR/Cas endonuclease and one or more guide nucleic acids (gNAs), one or more zine finger nucleases, one or more Transcription Activator-Like Effector Nucleases (TALENs), or one or more CRE recombinase, to induce the double strand breaks. In some embodiments, the CRISPR/Cas endonuclease comprises CasI, CasIB, Cas2, Cas3, Cas4, Cas5, Cas6, Cas7, Cas8, Cas9, Cas10, CasX, CasY, Cpf1 (Cas12a), Cas12b, Cas13a, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, CsbI, Csb2, Csb3, Csx17, Csx14, Csx10, Csx16, CsaX, Csx3, Csx1, Csx15, Csf1, Csf2, Csf3, Csf4, Cms1, C2c1, C2c2, or C23, or a homolog, ortholog or modified version thereof. In some embodiments, the CRISPR/Cas endonuclease comprises Cas9, Cpf1 (Cas12a), Cas12b, CasX, CasY, C2c1, or C2c3, or a homolog, ortholog, or modified version thereof. In some embodiments, the CRISPR/Cas endonuclease comprises Cas9. In some embodiments, the gNA comprises a single guide RNA (sgRNA).

In some embodiments, the target chromosome comprises, from 5′ to 3′, the sequence of the 5′ homology arm of the first nucleic acid molecule, the target sequence, and the sequence of 3′ homology arm of the second nucleic acid molecule. In some embodiments, the template chromosome comprises, from 5′ to 3′, the sequence of the 3′ homology arm of the first nucleic acid molecule, the template sequence, and the sequence of the 5′ homology arm of the second nucleic acid molecule.

In some embodiments, the target sequence comprises at least 1 gene, at least 2 genes, at least 3 genes, at least 5 genes, at least 10 genes, at least 20 genes, at least 30 genes, at least 40 genes, at least 50 genes, at least 100 genes, or at least 200 genes. In some embodiments, the target sequence comprises one or more genes that are homologous to one or more genes of the template sequence.

In some embodiments, the template sequence comprises a naturally occurring sequence. In some embodiments, the template sequence comprises at least 1 gene, at least 2 genes, at least 3 genes, at least 5 genes, at least 10 genes, at least 20 genes, at least 30 genes, at least 40 genes, at least 50 genes, at least 100 genes, or at least 200 genes. In some embodiments, the template sequence comprises one or more modifications to the naturally occurring sequence. In some embodiments, the template sequence comprises an artificial sequence. In some embodiments, the artificial sequence comprises a sequence encoding one or more antibodies or antigen binding fragments thereof. In some embodiments, the one or more antibodies or antigen binding fragments thereof comprise an scFv, a bi-specific antibody, or a multi-specific antibody.

In some embodiments, the target sequence is deleted by the insertion of the template sequence. In some embodiments, (a) the target chromosome comprises, from 5′ to 3′, the sequence of the 5′ homology arm of the first nucleic acid molecule, a first sgRNA target sequence, the target sequence, a second sgRNA target sequence, and the sequence of 3′ homology arm of the second nucleic acid molecule; and (b) the template chromosome comprises, from 5′ to 3′, a third sgRNA target sequence, the sequence of the 3′ homology arm of the first nucleic acid molecule, the template sequence, the sequence of the 5′ homology arm of the second nucleic acid molecule, and a fourth sgRNA target sequence. In some embodiments, generating the double stranded breaks comprises contacting the cell with a CRISPR/Cas endonuclease, and the first, second, third, and fourth sgRNAs. In some embodiments, the first, second, third, and fourth sgRNAs comprising targeting sequences specific to the first, second, third, and fourth sgRNA target sequences.

In some embodiments, contacting the cell with the CRISPR/Cas endonuclease and the sgRNAs comprises transfecting the cell with one or more nucleic acid molecules encoding the CRISPR/Cas endonuclease and the sgRNAs.

In some embodiments, inserting the template sequence comprises little or no deletion of a sequence of the target sequence. In some embodiments, inserting the template sequence disrupts one or more functions of the target sequence. In some embodiments, inserting the template sequence disrupts a gene in the target sequence. In some embodiments (a) the target chromosome comprises, from 5′ to 3′, the sequence of the 5′ homology arm of the first nucleic acid molecule, a first sgRNrA target sequence, and the sequence of 3′ homology arm of the second nucleic acid molecule; and (b) the template chromosome comprises, from 5′ to 3′, a second sgRNA target sequence, the sequence of the 3′ homology arm of the first nucleic acid molecule, the template sequence, the sequence of the 5′ homology arm of the second nucleic acid molecule, and a third sgRNA target sequence. In some embodiments, generating the double stranded breaks comprises contacting the cell with a CRISPR/Cas endonuclease, and a first, second, and third sgRNA. In some embodiments, the first, second, and third sgRNAs comprising targeting sequences specific to the first, second, and third sgRNA target sequences. In some embodiments, contacting the cell with the CRISPR/C as endonuclease and the sgRNAs comprises transfecting the cell with one or more nucleic acid molecules encoding the CRISPR/Cas endonuclease and the sgRNAs.

In some embodiments, the first or second marker comprises a fluorescent protein operably linked to a promoter capable of expressing the fluorescent protein in the cell. In some embodiments, the fluorescent protein comprises green fluorescent protein (GFP), yellow fluorescent protein (YFP), red fluorescent protein (RFP), cyan fluorescent protein (CFP), blue fluorescent protein (BFP), dsRed, mCherry, or tdTomato. In some embodiments, the fluorescent protein comprises GFP. In some embodiments, the first marker further comprises a selectable marker. In some embodiments, the second marker further comprises a selectable marker. In some embodiments, the selectable marker is selected from the group consisting of Dihydrofolate reductase (DHFR), Glutamine synthase (GS), Puromycin acetyltransferase, Blasticidin deaminase, Histidinol dehydrogenase, Hygromycin phosphotransferase (hph), Bleomycin resistance gene and Arninoglycoside phosphotransferase (Neomycin resistance). In some embodiments, the first and second markers are not the same selectable marker. In some embodiments, the first marker comprises GFP operably linked to a promoter capable of expressing the GFP in the cell and Puromycin acetyltransferase, and the second marker comprises Hygromycin phosphotransferase.

In some embodiments, the methods further comprise (e) deleting all or a part of the first or second marker after step (d). In some embodiments, deleting the first or second marker comprises inducing a deletion with a CRISPR/Cas endonuclease and a gNA comprising a targeting sequence specific to the sequence encoding the marker.

In some embodiments, the cells comprise hybrid cells, embryonic hybrid stem (EHS) cells or zygotes. In some embodiments, the EFIS cells are generated by fusing ES cells from any two species selected from the group consisting of mouse, rat, rabbit, guinea pig, hamster, sheep, goat, donkey, cow, horse, camel, chicken and monkey. In some embodiments, the EHS cells are generated by fusing human embryonic stem cells to embryonic stem cells from a non-human species. In some embodiments, the non-human species is mouse, rat, rabbit, guinea pig, hamster, sheep, goat, donkey, cow, horse, camel, chicken or monkey. In some embodiments, the EHS cells are generated by fusing EH cells from any two different species selected from the group consisting of mouse, rat, rabbit, guinea pig, hamster, sheep, goat, donkey, cow, horse, camel, chicken and monkey. In some embodiments, the fusion comprises electrofusion, viral induced fusion, or chemically induced fusion.

In some embodiments, the cells comprise hybrid cells. In some embodiments, generating the hybrid cells comprises: (a) generating micronucleated human cells; and (b) fusing the micronucleated human cells with a cell from a non-human species, thereby generating a hybrid cell. In some embodiments, the micronucleated human cells are generated by exposing human cells colcemid under conditions sufficient to induce micronucleation and collecting the micronucleated cells using centrifugation. In some embodiments, the non-human species is mouse, rat, rabbit, guinea pig, hamster, sheep, goat, donkey, cow, horse, camel, chicken or monkey. In some embodiments, the cell from the non-human species is an ES cell, and the hybrid cell is an EHS cell.

In some embodiments, the target sequence comprises a gene encoding an immunoglobulin or a T cell receptor subunit. In some embodiments, the target chromosome comprises mouse chromosome 12 and the template chromosome comprises human chromosome 14. In some embodiments, the target sequence comprises a mouse Igh variable region sequence.

In some embodiments, the mouse Igh variable region sequence comprises a sequence encoding mouse VH, DH and JH1-6 gene segments and intervening non-coding sequences. In some embodiments, the template sequence comprises a human IGH variable region sequence. In some embodiments, the human IGH variable region sequence comprises a sequence encoding human VH, DH and JH1-6 gene segments and intervening non-coding sequences. In some embodiments, the target sequence comprises a mouse Igl variable region sequence. In some embodiments, the target sequence comprises a mouse Igk variable region sequence. In some embodiments, the template sequence comprises a human IGL variable region sequence. In some embodiments, the template sequence comprises a human IGK variable region sequence. In some embodiments, the mouse Igk variable region sequence comprises a sequence encoding mouse Vₖ, and Jₖ₁₋₅ gene segments and intervening non-coding sequences. In some embodiments, the template sequence comprises a human IGK variable region sequence. In some embodiments, the human IGK variable region sequence comprises a sequence encoding human Vₖ, and Jₖ₁₋₅ gene segments and intervening non-coding sequences.

In some embodiments, the methods further comprise recovering the engineered chromosome from the cells selected at step (d). In some embodiments, recovering the engineered chromosome comprises exposing the cells to colcemid under conditions sufficient to induce micronucleation and collecting micronucleated cells using centrifugation.

In some embodiments, the first and second nucleic acid molecules are plasmids.

The disclosure provides engineered chromosomes produced by the methods of the disclosure.

In some embodiments, the engineered chromosome is a mouse chromosome 12 comprising a sequence of a human IGH variable region in place of a mouse Igh variable region. In some embodiments, the mouse Igh variable region comprises VH, DH and JH1-6 gene segments and intervening non-coding sequences. In some embodiments, the human IGH variable region comprises VH, DH and JH1-6 gene segments and intervening non-coding sequences. In some embodiments, the engineered chromosome is a mouse chromosome 6 comprising a sequence of a human IGK variable region in place of a mouse Igk variable region. In some embodiments, the mouse Igk variable region sequence comprises a sequence encoding mouse Vₖ, and Jₖ₁₋₅ gene segments and intervening non-coding sequences. In some embodiments, the template sequence comprises a human IGK variable region sequence. In some embodiments, the human IGK variable region sequence comprises a sequence encoding human Vₖ, and Jₖ₁₋₅ gene segments and intervening non-coding sequences.

The disclosure provides cells comprising the engineered chromosomes of the disclosure.

In some embodiments, the cells are capable of hybridizing with a mouse ES cell. In some embodiments, the cells are embryonic stem (ES) cells, embryonic hybrid stem (EHS) cells, or zygotic cells. In some embodiments, the EHS cell is a hybrid of human and mouse ES cells. In some embodiments, the ES cell is a mouse ES cell. In some embodiments, the cell is a micronucleated cell.

The disclosure provides methods comprising generating a mouse embryonic stem cell, comprising: (a) fusing micronucleated cells comprising the engineered chromosome produced by the methods of any one of methods of the disclosure to mouse ES cells, wherein: (i) the mouse ES cells comprise a chromosome homologous to the engineered chromosome, the homologous chromosome comprising a first fluorescent protein operably linked to a promoter capable of expressing the fluorescent protein in the ES cells, and (ii) at least a subset of the micronucleated cells comprise the engineered chromosome, and wherein the engineered chromosome comprises a second fluorescent protein different from the first fluorescent protein, the second fluorescent protein operably linked to a promoter capable of expressing the fluorescent protein in the ES cells; (b) selecting ES cells that express both the first and second fluorescent proteins; (c) culturing the ES cells selected in step (c) until the homologous chromosome is lost by at least a subset of the ES cells; and (d) selecting ES cells that express the second fluorescent protein and do not express the first fluorescent protein.

In some embodiments, culturing the cells at step (c) comprises culturing the cells for at least 5 days, at least 7 days, at least 10 days, or at least 14 days. In some embodiments, selecting the cells at steps (b) and (d) comprises fluorescence activated cell sorting (FACS).

The disclosure provides mouse ES cells produced by the methods of the disclosure.

The disclosure provides a transgenic mouse produced from the mouse ES cells of the disclosure.

In some embodiments, producing the transgenic mouse comprises injecting the ES cell into a diploid blastocyst, nuclear transfer from the ES cell to an enucleated mouse embryo, or tetraploid embryo complementation. In some embodiments, mouse chromosome 12 comprises a sequence of a human IGH variable region in place of a mouse Igh variable region. In some embodiments, the mouse Igh variable region comprises VH, DH and JH1-6 gene segments and intervening non-coding sequences. In some embodiments, the human IGH variable region comprises VH, DH and 11-6 gene segments and intervening non-coding sequences. In some embodiments, mouse chromosome 6 comprises a sequence of a human IGK variable region in place of a mouse Igk variable region. In some embodiments, the mouse Igk variable region sequence comprises a sequence encoding mouse Vₖ, and Jₖ₁₋₅ gene segments and intervening non-coding sequences. In some embodiments, the template sequence comprises a human IGK variable region sequence. In some embodiments, the human IGK variable region sequence comprises a sequence encoding human Vₖ, and Jₖ₁₋₅ gene segments and intervening non-coding sequences.

The disclosure provides methods of generating an antibody comprising: (a) challenging the transgenic mouse of the disclosure with an antigen, whereby the transgenic mouse generates a plurality of antibodies comprising human V, D, and J segments from the human IGH variable region; and (b) isolating an antibody specific to the antigen.

The disclosure provides methods of generating an antibody comprising: (a) challenging the transgenic mouse of the disclosure with an antigen, whereby the transgenic mouse generates a plurality of antibodies comprising human V, and J segments from the human IGK or IGL variable region; and (b) isolating an antibody specific to the antigen.

The disclosure provides antibodies derived from the antibody produced by the transgenic mouse of the disclosure. In some embodiments, the antibody comprises a single chain variable fragment (scFv)., bispecific antibody or multi-specific antibody.

The disclosure provides methods of generating a chromosomal rearrangement, comprising: (a) providing a cell comprising a target chromosome comprising a target location and a template chromosome comprising a template sequence; (b) contacting the cell with a nucleic acid molecule comprising from 5′ to 3′, a 5′ homology arm comprising a nucleotide sequence upstream of the 5′ end of the target location, a marker, and a 3′ homology arm comprising a nucleotide sequence upstream of the 5′ end of the template sequence; (c) generating double strand breaks at the target location, and at the 5′ end of the template sequence, whereby the marker is inserted in the target chromosome 3′ of the sequence of the 5′ homology arm, followed by the template sequence, thereby generating a chromosomal rearrangement; and (d) selecting a cell or cells expressing the marker.

In some embodiments, the 5′ and 3′ homology arms of the nucleic acid molecule are between about 20 and 2,000 bp, between about 50 and 1,500 bp, between about 100 and 1,400 bp, between about 150 and 1,300 bp, between about 200 and 1,200 bp, between about 300 and 1,100 bp, between about 400 and 1,000 bp, or between about 500 and 900 bp, or between about 600 bp and 800 bp in length. In some embodiments, the 5′ and 3′ homology arms of the nucleic acid molecule are between about 400 and 1,500 bp in length, between about 500 and 1,300 bp in length, or between about 600 and 1,000 bp in length. In some embodiments, 5′ and 3′ homology arms of the nucleic acid molecule are between about 600 and 1,000 bp in length.

In some embodiments, generating the double strand breaks at (c) comprises using a CRISPR/Cas endonuclease and at least one sgRNA, one or more zinc finger nucleases, one or more Transcription Activator-Like Effector Nucleases (TALENs), or one or more CRE recombinase to induce the double strand breaks. In some embodiments, the CRISPR/Cas endonuclease comprises CasI, CasIB, Cas2, Cas3, Cas4, Cas5, Cas6, Cas7, Cas8, Cas9, Cas10, CasX, CasY, Cas12a (Cpf1), Cas12b, Cas13a, Csy1, Csy2, Csy3, Cse1, Cse2, CscI, Csc2, Csa5, Csn2, Csm2, Csm3, Csm4, Csm5, Csm6, Cmr1, Cmr3, Cmr4, Cmr5, Cmr6, CsbI, Csb2, Csb3, Csx17, Csx14, Csx10, Csx16, CsaX, Csx3, Csx1, Csx15, Csf1, Csf2, Csf3, Csf4, Cms1, C2c1, C2c2, or C2c3, or a homolog, ortholog, or modified version thereof. In some embodiments, the CRISPR/Cas endonuclease comprises Cas9, Cpf1, CasX, CasY, C2c1, or C2c3, or a homolog, ortholog or modified version thereof. In some embodiments, the CRISPR/Cas endonuclease comprises Cas9. In some embodiments, generating the double stranded breaks comprises contacting the cell with a CRISPR/Cas endonuclease, at least a first gNA comprising a targeting sequence specific to the target location, such that the CRISPR/Cas endonuclease cleaves the target location, and a second gNA comprising a targeting sequence specific to the 5′ end of the template sequence. In some embodiments, contacting the cell with the CRISPR/Cas endonuclease and the sgRNAs comprises transfecting the cell with one or more nucleic acid molecules encoding the CRISPR/Cas endonuclease and the sgRNAs. In some embodiments, the one or more nucleic acid molecules are plasmids.

In some embodiments, the marker comprises a fluorescent protein operably linked to a promoter capable of expressing the fluorescent protein in the cell. In some embodiments, the fluorescent protein comprises GFP, YFP, RFP, CFP, BFP, dsRed, mCherry, or tdTomato. In some embodiments, the marker further comprises a selectable marker. In some embodiments, the selectable marker is selected from the group consisting of Dihydrofolate reductase (DHFR), Glutamine synthase (GS), Puromycin acetyltransferase, Blasticidin deaminase, Histidinol dehydrogenase, Hygromycin phosphotransferase (hph), Bleomycin resistance gene, and Aminoglycoside phosphotransferase (Neomycin resistance).

In some embodiments, the cells comprise embryonic stem (ES) cells.

In some embodiments, the nucleic acid molecule is a plasmid.

The disclosure provides cells comprising the chromosomal rearrangement produced by the methods of the disclosure. In some embodiments, the cell is a mouse ES cell.

The disclosure provides transgenic mice, from the mouse ES cell produced by the methods of the disclosure.

### BRIEF DESCRIPTION OF THE DRAWINGS

A better understanding of the features and advantages of the present disclosure will be obtained by reference to the following detailed description that sets forth illustrative embodiments, and the accompanying drawings, of which:

FIG. 1 is a diagram that shows, from top to bottom, the mouse immunoglobulin heavy chain complex (Igh), human Igh, and a mouse Igh in which the variable domains (V_{H}, DH and J_{H}1-6) have been humanized. Chro: chromosome.

FIG. 2 is a diagram showing the hybridization of engineered mouse and human embryonic stem (ES) cells via electrofusion. Mouse ES cells express the marker neomycin, and human ES express mCherry. Embryonic hybrid stem cells (hybridoma cells) are resistant to G418 and positive for mCherry.

FIG. 3A is a diagram showing the placement of three pairs of PCR primers (shown as arrows) in the human Igh gene V_{H}, DH and JH1-6 regions, which were used to genotype embryonic hybrid stem (EHS) cells.

FIG. 3B is an exemplary gel showing PCR results for 12 embryonic hybrid stem (EHS) cell clones that were genotyped using the primers shown in FIG. 3A.

FIGS. 4A-4B are diagrams showing the pipeline for establishing an engineered, humanized chromosome in an EHS cell (FIG. 4A) via HDR-mediated chromosomal rearrangement (HCMR) HDR: homology directed repair. EHS cells were co-transfected with a 5′ HMCR plasmid containing a 5′ arm homologous to the 5′ of the mouse Igh gene, a 3′ am-homologous to the 5′ of the human Igh gene, and a pCMV-EGFP-polyA-PGK-Puromycin-polyA cassette; a 3′ HMCR plasmid containing a 5′ arm homologous to the 3′ end of the human Igh variable loci, a 3′ arm homologous to the 3′ of mouse Igh variable loci and a PGK-Hygromycin-polyA cassette, and four plasmids containing Cas9 and sgRNAs targeting the 5′ and 3′ variable domains of mouse Igh and human Igh, as shown by ( [symbol] ). Or (FIG. 4B) via CRE-Loxp mediated chromosome rearrangement (CMCR): Four plasmids were designed to mediate the CMCR process. The mouse Igh 5′ (pCMV-GFP-BGH PolyA-Loxp) and 3′ (BGH polyA-Loxp-511-Hygromycin-BGH polyA-PGK-BSD-BGH PolyA) plasmids were designed to insert into 5′ and 3′ end of the mouse Igh variable loci, respectively. Simultaneously, the human IGH 5′ (BGH polyA-Loxp-Puro-BGH PolyA-PGK-Neomycin-BGH PolyA) and 3′ (pCMV-BGP-BGH PolyA-PGK-Loxp-511) plasmids were designed to insert into 5′ and 3′ end of the human IGH variable loci, respectively. Cre was transfected into the successfully integrated EHS cells for CMCR.

FIG. 5A is a diagram showing the placement of PCR primers (shown as arrows) used to validate the engineered human chromosome.

FIG. 5B shows the PCR results using the four pairs of primers listed in FIG. 5A. Results for 192 single clones are shown.

FIG. 6 is a diagram showing replacement of a mouse chromosome with the engineered human chromosome in mouse ES cells. EHS cells carrying the engineered human chromosome marked with GFP are micronized through exposure to colcemid, the microcells are collected by centrifugation, and electrofused to mouse ES cells in which the corresponding mouse chromosome has been marked with mCherry. GFP+ mCherry+ cells are isolated by fluorescence activated cell sorting (FACS). Cells are then cultured, and GFP+ mCherry-cells that have lost the mouse chromosome are isolated by FACS.

FIG. 7A shows the placement of PCR primers (shown as arrows) used to validate Igh humanized mice.

FIG. 7B shows the PCR results for an exemplary Igh humanized mouse using the 7 pairs of primers shown in FIG. 7A.

FIG. 8A shows fluorescent in situ hybridization (FISH) results for an Igh humanized mouse.

FIG. 8B shows G-banding karyotype analysis for an Igh humanized mouse.

FIG. 9A shows whole genome sequencing (WGS) analysis of TGH-V of Igh humanized mice. Copy numbers of WGS sequences for each variable (V) gene segment located on the VH region of human Igh are shown.

FIG. 9B shows WGS analysis of IGH-D and IGH-J of Igh humanized mice. Copy numbers of WGS sequences for each Diversity (D) gene segment and the 6 joining (J) segments located in the located on the D_{H} and J_{H}1-6 regions of human Igh are shown.

FIG. 10 shows humanization of the variable domains of mouse Igk gene.

FIGS. 11A-11B show PCR validation result of Igk humanized mice. FIG. 11A, Location of the design primers used for PCR experiments. FIG. 11B, PCR result using 5 pairs of primers listed in panel A for Igk humanized mice.

FIG. 12 shows WGS analysis result of Igk humanized mice. Copy numbers from WGS sequences for each antibody genes located on the V_{K} and Jₖ segments of human IGK gene.

### DETAILED DESCRIPTION

The present disclosure provides methods for engineering chromosomes comprising transferring large fragments of sequence between chromosomes. Using the methods disclosed herein, sequences of at least 5 Megabasepairs (MB) can be transferred to a target chromosome from a chromosomal template. The methods disclosed herein can also be used to generate chromosomal rearrangements, such as inversions and translocations. Also provided herein are engineered chromosomes produced by the methods of the disclosure, as well as cells and animals comprising these engineered chromosomes, and methods of using same.

Manipulation of large fragments of genes or chromosomes holds great promise for both basic and translational research as well as development of therapies. Genetic humanization is one of the most popular applications, where genes of a model organism, such as a mouse, are replaced with their human counterparts. For example, mice carrying humanized Ig genes provide as a powerful platform for the production of human antibodies in a mouse background. However, large fragment manipulation remains one of the most significant challenges in the gene editing field, as delivery vectors able to carry large fragments of chromosome up to million base pairs (MBs) are not available. The payload of conventional delivery vectors, such as adeno-associated viral vectors or other viral vectors, are limited by the size viral genome from which the vector is derived.

The methods disclosed herein allow for the efficient in situ replacement of large sequences between chromosomes. These methods, termed Massive fragment Across Species In situ Replacement Technology (MASIRT), can be used to replace large portions of chromosomes in a single editing step, in some cases up to megabasepairs (MB) of sequence. These methods can be used to efficiently transfer large sequences between species, or between chromosomes within a single species. In one example, MASIRT was used to obtain mice humanized for the variable domains of the mouse Igh gene. Human and mice show high similarity in the arrangement and expression of antibody genes, and the genomic organization of the heavy chains are also similar between these species. Therefore, a humanized mouse Igh gene was obtained using MASIRT to replace approximately 3 MB of mouse genomic sequences containing all V_{H}, D_{H}, and J_{H} gene segments with the approximately 1 MB of contiguous human genomic sequence containing the equivalent human gene fragments.

Unlike other methods that only work on embryonic stem cells, the methods of the instant disclosure can advantageously be used to replace large sequences in zygotes. Embryonic stem cell lines are not generally available for species other than mice. In contrast, zygotes are available to many mammals, therefore the methods of the instant disclosure can be used to obtain animals such as rabbits or cow with humanized for genes or gene fragments. In addition, the methods disclosed herein can be used to replace large sequence fragments, e.g., sequences up to at least 5 MB, at one time, about five times larger than the methods used by other methods known in the art. This increases the efficiency, and reduces the time and cost needed to create animals with humanized genes. For example, Igh humanized mice can be made with only 3 rounds of replacement. A further advantage is that, when used in mice, each replacement takes only 1-3 months, which is only one-half or one-third the amount of time needed for other methods known in the art.

### Definitions

A chromosome is a long DNA molecule that contains all or part of the genetic material of an organism. Most eukaryotic chromosomes include packaging proteins called histones which, aided by chaperone proteins, bind to and condense the DNA molecule to maintain its integrity. Eukaryotic chromosomes consist of a long linear DNA molecule associated with proteins, forming a compact complex of proteins and DNA called chromatin. Each chromosome has one centromere, with one or two arms projecting from the centromere. The arms of the chromosome end in telomeres, which are region of repetitive nucleotide sequences associated with specialized proteins, and which protect the terminal regions of chromosomal DNA from progressive degradation and ensure the integrity of linear chromosomes by preventing DNA repair systems from mistaking the very ends of the DNA strand for a double strand break.

A “gene” includes a DNA region encoding a gene product (e.g., a protein, or a non-coding RNA), as well as all DNA regions which regulate the production of the gene product, whether or not such regulatory sequences are adjacent to coding and/or transcribed sequences. Accordingly, a gene may include regulatory element sequences including, but not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Coding sequences encode a gene product upon transcription or transcription and translation. The coding sequences of the disclosure may comprise fragments and need not contain a full-length open reading frame. A gene can include both the strand that is transcribed as well as the complementary strand containing the anticodons. A gene may also include exons, which can include protein coding sequences and untranslated regions, as well as introns, which are removed from the final RNA product by splicing.

The term “promoter” as used herein can refer to a DNA sequence that is located adjacent to a DNA sequence that encodes a recombinant product. A promoter is preferably linked operatively to an adjacent DNA sequence. A promoter typically increases an amount of protein or RNA product expressed from a DNA sequence as compared to an amount expressed when no promoter exists. A promoter from one organism can be utilized to enhance protein expression from a DNA sequence that originates from another organism. For example, a vertebrate promoter may be used for the expression of jellyfish GFP in vertebrates. In addition, one promoter element can increase an amount of recombinant products expressed for multiple DNA sequences attached in tandem. Hence, one promoter element can enhance the expression of one or more recombinant products. Multiple promoter elements are well-known to persons of ordinary skill in the art.

The term “enhancer” as used herein can refer to a DNA sequence that is located adjacent to the DNA sequence that encodes the protein or RNA product, or that is located distal from the DNA sequence that encodes the protein or RNA product. Enhancer elements are typically located upstream of a promoter element, but can be located downstream of or within a coding DNA sequence, such as within an intron. In some cases, an enhancer can be located kilobases or even tens or hundreds of kilobases from the gene whose expression it regulates. Enhancer elements can increase an amount of protein or RNA product expressed from a DNA sequence above increased expression afforded by a promoter element. Multiple enhancer elements are readily available to persons of ordinary skill in the art.

As used herein, the term “exogenous chromosome” or “exogenous sequence” refers to a foreign chromosome or foreign sequence with respect to the genome of an animal. For example, in a mouse cell, in which all chromosomes are mouse chromosomes except for a single human chromosome, the human chromosome is an exogenous chromosome. Similarly, in a mouse chromosome in which a portion of the mouse sequence has been replaced with human sequence, the human sequence is referred to as exogenous sequence. Similarly, “endogenous” refers to a chromosome or sequence originating from the organism, such as the mouse chromosomes or sequences described supra.

As used herein, the term “homologous recombination” refers to a type of genetic recombination in which nucleotide sequences are exchanged between two similar or identical molecules of DNA known as homologous sequences or homology arms. Homologous recombination often involves the following basic steps: after a double-strand break (DSB) occurs on both strands of DNA, sections of DNA around the 5′ ends of the DSB are cut away in a process called resection. In the strand invasion step that follows, an overhanging 3′ end of the broken DNA molecule “invades” a similar or identical (or homologous) DNA molecule, e.g., a homology arm, that is not broken. After strand invasion, the further sequence of events may follow either of two pathways the DSBR (double-strand break repair) pathway or the SDSA (synthesis-dependent strand annealing) pathway.

“DNA repair pathway,” as used herein, refers to the cellular mechanisms that allow a cell to maintain genome integrity function, in response to the detection of DNA damage, such as single or double-stranded breaks in the DNA. Depending on the type and extent of DNA damage, and cell cycle phase, the DNA repair pathway can include, but is not limited to, pathways such as resection, canonical homology directed repair (canonical HDR), homologous recombination (HR), alternative homology directed repair (alt-HDR), double-strand break repair (DSBR), single-strand annealing (SSA), synthesis-dependent strand annealing (SDSA), break-induced replication (BIR), alternative end-joining (alt-EJ), microhomology mediated end-joining (MMEJ), DNA synthesis-dependent microhomology-mediated end-joining (SD-MMEJ), non-homologous end joining (NIHEJ) pathways such as canonical non-homologous end-joining (C-NHEJ) repair, alternative non-homologous end joining (A-NHEJ) pathway, translesion DNA synthesis (TLS) repair, base excision repair (BER), nucleotide excision repair (NER), mismatch repair (MMR), DNA damage responsive (DDR), Blunt End Joining, single strand break repair (SSBR), interstrand crosslink repair (ICL) and Fanconi Anemia pathway (FA).

As used herein, homology directed repair (HDR) refers to the process of repairing DNA damage using a homologous nucleic acid (e.g., a sister chromatid or an exogenous nucleic acid). In a normal cell, HDR typically involves a series of steps such as recognition of the break, stabilization of the break, resection, stabilization of single stranded DNA, formation of a DNA crossover intermediate, resolution of the crossover intermediate, and ligation.

As used herein a “homolog” refers a protein in a group of proteins that perform the same biological function, e.g. proteins that belong to the same protein family and that provide a common trait or perform the same or a similar biological function. Homologs are expressed by homologous genes. Homologous genes are genes which encode proteins with the same or similar biological function to the protein encoded by the second gene. Homologous genes can be generated by the event of speciation (orthologs) or by the event of genetic duplication (paralogs). “Orthologs” refer to a set of homologous genes in different species that evolved from a common ancestral gene by speciation. Normally, orthologs retain the same function in the course of evolution. “Paralogs” refer to a set of homologous genes in the same species that have diverged from each other as a consequence of genetic duplication. Thus, homologous genes can be from the same or a different organism. Homologous genes include naturally occurring alleles and artificially-created variants. The percent identity between homologous proteins will depend on the source of the proteins, and the degree to which the species from which the proteins are derived have diverged. Homologous proteins from more closely related species (e.g., two mammals such as human and mouse) will generally be more similar than proteins from more distantly related species (e.g., chicken and mouse). When optimally aligned, homologous proteins have typically at least about 40% identity, about 50% identity, about 60% identity, in some instances at least about 70%, for example about 80% and even at least about 90% identity over the full length of the protein. In other cases, for example when comparing proteins from highly divergent species, homologous proteins will have at least about 40% identity, about 50% identity, about 60% identity, about 70%, about 80% identity or about 90% identity over the length of a conserved protein domain, such as a DNA binding domain.

Homologous genes or proteins are identified by comparison of DNA or amino acid sequence, e.g. manually or by use of a computer-based tool using known homology-based search algorithms such as those commonly known and referred to as BLAST, FASTA, and Smith-Waterman. A local sequence alignment program, e.g. BLAST, can be used to search a database of sequences to find similar sequences, and the summary Expectation value (E-value) used to measure the sequence base similarity. Because a protein hit with the best E-value for a particular organism may not necessarily be an ortholog, i.e. have the same function, or be the only ortholog, a reciprocal query can be used to filter hit sequences with significant-values for ortholog identification. The reciprocal query entails search of the significant hits against a database of amino acid sequences from the base organism that are similar to the sequence of the query protein. A hit can be identified as an ortholog, when the reciprocal query's best hit is the query protein itself or a protein encoded by a duplicated gene after speciation.

As used herein, “percent identity” means the extent to which two optimally aligned DNA or protein segments are invariant throughout a window of alignment of components, for example nucleotide sequence or amino acid sequence. An “identity fraction” for aligned segments of a test sequence and a reference sequence is the number of identical components that are shared by sequences of the two aligned segments divided by the total number of sequence components in the reference segment over a window of alignment which is the smaller of the full test sequence or the full reference sequence. “Percent identity” (“% identity”) is the identity fraction times 100. Such optimal alignment is understood to be deemed as local alignment of DNA sequences. For protein alignment, a local alignment of protein sequences should allow introduction of gaps to achieve optimal alignment. Percent identity can be calculated over the aligned length not including the gaps introduced by the alignment per se.

As used herein, “specific to”, when used in reference to a nucleotide sequence such as a homology arm or targeting sequence of a guide RNA, refers to a sequence that is identical, or substantially identical to, another nucleotide sequence or the reverse complement of the other nucleotide sequence. A sequence that is “specific to” another sequence is capable of hybridizing to the other sequence or its reverse complement through Watson-Crick base-pairing. Thus, the skilled artisan will appreciate that a sequence that is specific to another sequence is highly similar to the other sequence or its reverse complement, but need not be perfectly identical. For example, a sequence that is at least 80%, at least 85%, at least 90%, at least 95%, at least 97% or at least 99% identical to another sequence, is still specific to that sequence if it is capable of hybridizing to the other sequence. As a further example, a guide nucleic acid targeting sequence may comprise 1, 2, 3, or more mismatches to a target sequence, depending on the location of the mismatches in the targeting sequence, and yet is still specific to the target sequence if it is capable of targeting a ribonucleoprotein complex comprising the gNA and an endonuclease to the target sequence.

“Selecting,” as used herein, refers to separating two populations of distinct products using any methods known in the art. Selecting, as it applies to cells, chromosomes, or sequences, can be done on the basis of a marker, such as a selectable marker. Selecting cells expressing the selectable marker involves culturing a mixed population of cells including cells that express the marker and cell that do not express the marker in a selective medium, such that the cells that do not express the marker are killed or their growth is inhibited. Sequences or chromosomes comprising the marker can similarly be selected for by placing them within cells and applying a selective regimen. Similarly, selection can be done on the basis of a detectable marker, such a fluorescent protein. Cells expressing the detectable marker can be physically removed from a mixed population of cells on the basis of the detectable marker using methods known in the art, such as fluorescence activated cell sorting (FACS). Alternatively, or in addition, Alternatively, the mixed population of cells can be diluted such that single cells can be cultured in isolation, and clones derived from isolated cells assayed for the presence of one or more traits, such as a marker.

“Derived from”, as used herein, refers to the source or origin of a molecular entity, e.g., a nucleic acid or protein. The source of a molecular entity may be naturally-occurring, recombinant, unpurified, or a purified molecular entity. For example, a polypeptide that is derived from a second polypeptide may comprise an amino acid sequence that is identical or substantially similar, e.g., is more than 50% homologous to, the amino acid sequence of the second protein. The derived molecular entity, e.g., a nucleic acid or protein, can comprise one or more modifications, e.g., one or more amino acid or nucleotide changes.

“Isolated from” refers to a molecular entity that has been purified, removed or isolated from its source or origin.

A “naturally occurring” sequence is one that is found in at least one species found in nature.

An “artificial sequence” refers to a sequence that is not found in nature. Artificial sequences may be similar to naturally occurring sequences, but contain one or more alterations relative to their naturally occurring counterpart. Alternatively, artificial sequences may bear little or no similarity to any naturally occurring sequence. Chimeric, or recombinant sequences, in which two sequences from disparate sources, or which are never found adjacent to each other, are operably linked together, are a type of artificial sequence.

“Operatively linked” or “operably linked” refers to a juxtaposition of genetic elements, wherein the elements are in a relationship permitting them to operate in the expected manner. For instance, a promoter is operatively linked to a coding region if the promoter helps initiate transcription of the coding sequence. There may be intervening residues between the promoter and coding region so long as this functional relationship is maintained.

The following classifications are used herein to refer to stein cells. The most pluripotent and earliest in terms of developmental stage, are the “embryonic stem (ES) cells” or “ES cells.” ES cells may be freshly derived primary cells, or from an ES cell-line. All other stem cells from somatic tissue (every tissue excluding germ cell tissue) are defined in general terms as “somatic stem cells”, but might be commonly known as any or all of the following: “adult stem cells”, “mature stem cells”, “progenitor cells”, “progenitor stem cells”, “precursor cells” and “precursor stem cells.” The other class of non-ES cell is defined as “germ line stem cells”. Finally, non-stem cells are herein described as “mature cells”, but are also known as “differentiated cells”, “mature differentiated cells”, “terminally differentiated cells” and “somatic cells.” Mature cells may also be primary isolated cells derived from tissue or an immortal cell line or a tumor-derived cell-line. The present disclosure further encompasses “precursor forms of a mature cell” which includes all cells that do not fulfil commonly used scientific definitions for either stem cells or mature cells. An ES cell can be cultured for an extended period in vitro, and, before it is inserted/injected into the cavity of a normal blastocyst, be induced to resume a normal program of embryonic development to differentiate into all cell types of an adult animal, including germ cells.

As used herein, a “hybrid cell” refers to a cell that contains elements from two genomes. The skilled artisan will appreciate that a hybrid cell can contain two complete, or nearly complete genomes from separate sources. Alternatively, a hybrid cell may contain a complete genome from one source, and only a few chromosomes, a single chromosome, or part of a single chromosome, from a second source. A cell containing any mixture of elements from two genomes between the two extremes described supra is still considered a hybrid cell. The two genomes in the hybrid can come from different individuals, different strains of the same species, or different species. Hybrid cells can be generated by any method known in the art. These include, but are not limited to cell fusion, and microcell-mediated chromosome transfer (MMCT), which transfers small numbers of chromosomes from one cell to another.

As used herein, a “hybrid embryonic stem (EHS)” cell refers to a hybrid cell with embryonic stem cell properties. EHS cells can be generated by the fusion of ES cells from two different species, or through MMCT mediated chromosomal transfer of chromosomes from a cell of one species to a stem cell of another species.

“Cancer” as used herein refers to a disease, condition, trait, genotype or phenotype characterized by unregulated cell growth or replication as is known in the art. Cancers include both solid tumors and liquid tumors. Exemplary cancers include, but are not limited to, leukemias, breast cancers, bone cancers, brain cancers, cancers of the head and neck, cancers of the retina, cancers of the esophagus, gastric cancers, multiple myeloma, ovarian cancer, uterine cancer, thyroid cancer, testicular cancer, endometrial cancer, melanoma, colorectal cancer, lung cancer, bladder cancer, prostate cancer, lung cancer (including both small cell and non-small cell lung cancers), pancreatic cancer, sarcomas, carcinomas cervical cancer, head and neck cancers, and skin cancers.

All publications, patents, and patent applications mentioned in this specification are herein incorporated by reference to the same extent as if each individual publication, patent, or patent application was specifically and individually indicated to be incorporated by reference.

### Methods of Engineering Chromosomes

The disclosure provides methods of engineering chromosomes using a template chromosome, a target chromosome, one or more nucleic acid molecules such as vectors or plasmids, and homology directed repair. Nucleases are used to generating double stranded breaks flanking a template sequence in the template chromosome, and flanking the target sequence or at a target location in the target chromosome. One or more nucleic acid molecules comprising markers and homology arms comprising sequences of the target and template chromosomes are used to direct replacement of the target sequence with the template sequence, insertion of the template sequence at the target location, or creation of a chromosomal rearrangement by joining the target and template sequences at the site of the double strand break.

In some embodiments, the methods comprise replacing a target sequence with a template sequence, i.e. the target sequence is deleted by insertion of the template sequence.

In some embodiments, the methods comprise replacing a target sequence with a template sequence. Any suitable template sequence, and any suitable target sequence, may be used in the method described herein. For example, the methods can be used to replace part of a chromosome from a model organism with the homologous human sequence, thereby humanizing that part of the model organism's genome. Alternatively, a large sequence may be inserted at a target location with little or no deletion of the target sequence.

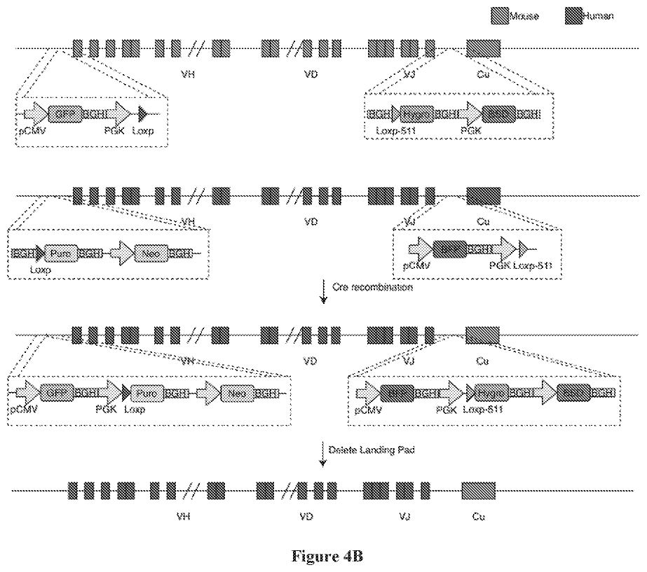

In some embodiments, the disclosure provides methods of generating an engineered chromosome, comprising: (a) providing a cell comprising a target chromosome comprising a target sequence and a template chromosome comprising a template sequence; (b) contacting the cell with (i) a first nucleic acid molecule comprising from 5′ to 3′, a 5′ homology arm comprising a nucleotide sequence upstream of the 5′ end of the target sequence, at least a first marker, and a 3′ homology arm comprising a nucleotide sequence upstream of the 5′ end of the template sequence; and (ii) a second nucleic acid molecule comprising from 5′ to 3′, a 5′ homology arm comprising a nucleotide sequence downstream of the 3′ end of the template sequence, at least a second marker, and a 3′ homology arm comprising a nucleotide sequence downstream of the 3′ end of the target sequence; (c) generating double strand breaks at or on either side of the target sequence, and at the 5′ and 3′ ends of the template sequence, whereby the template sequence and the first and second markers are inserted into the target chromosome; and (d) selecting a cell or cells expressing the first and second markers. In some embodiments, the first and/or second nucleic acid molecules are plasmids. The arrangement of template sequence, target sequence, and the homology arms of the first and second nucleic acid molecules for some embodiments of the methods described herein are shown in FIGS. 4A-4B. In some embodiments, the first marker is located at the 5′ end of the template sequence and the second marker is located at the 3′ end of the template sequence following insertion of the template sequence. For example, the engineered chromosome produced by the methods described herein comprises, from 5′ to 3′, after insertion of the template sequence and deletion of the target sequence, the target chromosomal sequence upstream of the target sequence, the first marker, the template sequence, the second marker, and the target chromosomal sequence downstream of the target sequence.

The skilled artisan will appreciate that template sequences of many lengths are suitable for the methods described herein. A suitable template sequence may be as small as a few hundred base pairs, or comprise most of a chromosome, and thus be up to several hundred megabasepairs in length. In some embodiments of the methods described herein, the template sequence is at least 25 KB, at least 50 KB, at least 100 KB, at least 200 KB, at least 400 KB, at least 500 KB, at least 600 KB, at least 700 KB, at least 800 KB, at least 900 KB, at least 1 MB, at least 2 MB, at least 3 MB, at least 4 MB, at least 5 MB, at least 10 MB, at least 15 MB, at least 20 MB, at least 50 MB, at least 100 MB, at least 150 MB, at least 200 MB, or at least 250 MB in length. In some embodiments, the template sequence is between 50 KB and 250 MB, 100 KB and 200 MB, 200 KB and 50 MB, 500 KB and 50 MB, 1 MB and 100 MB, 1 MB and 10 MB, 1 MB and 5 MB, 1 MB and 3 MB, 5 MB and 50 MB, 5 MB and 10 MB, 3 MB and 10 MB, or 5 MB and 50 MB, in length.

In some embodiments of the methods described herein, the template chromosome comprises, from 5′ to 3′, the sequence of the 3′ homology arm of the first nucleic acid molecule, the template sequence, and the sequence of the 5′ homology arm of the second nucleic acid molecule. In some embodiments, the template chromosome comprises, from 5′ to 3′, the sequence of the 3′ homology arm of the first nucleic acid molecule, a third endonuclease site, the template sequence, a fourth endonuclease site, and the sequence of the 5′ homology arm of the second nucleic acid molecule.

The skilled artisan will appreciate that target sequences of many lengths are suitable for the methods described herein. A suitable target sequence may be as small an endonuclease site used to generate double strand break (a target location), or comprise most of a chromosome, and thus be up to several hundred megabasepairs in length. In some embodiments of the methods described herein, the target sequence is at least 25 KB, at least 50 KB, at least 100 KB, at least 200 KB, at least 400 KB, at least 500 KB, at least 600 KB, at least 700 KB, at least 800 KB, at least 900 KB, at least 1 M13, at least 2 MB, at least 3 MB, at least 4 MB, at least 5 MB, at least 10 MB, at least 15 MB, at least 20 MB, at least 50 MB, at least 100 MB, at least 150 MB, at least 200 MB, or at least 250 MB in length. In some embodiments, the target sequence is between 50 KB and 250 MB, 100 KB and 200 MB, 200 KB and 50 MB, 500 KB and 50 MB, 1 MB and 100 MB, 1 MB and 10 MB, 1 MB and 5 MB, 1 MB and 3 MB, 5 MB and 50 MB, 5 MB and 10 MB, 3 MB and 10 MB, or 5 MB and 50 MB, in length.

In some embodiments of the methods described herein, the target chromosome comprises, from 5′ to 3′, the sequence of the 5′ homology arm of the first nucleic acid molecule, the target sequence, and the sequence of 3′ homology arm of the second nucleic acid molecule. In some embodiments, the target chromosome comprises, from 5′ to 3′, the sequence of the 5′ homology arm of the first nucleic acid molecule, a first endonuclease site, the target sequence, a second endonuclease site, and the sequence of 3′ homology arm of the second nucleic acid molecule.

In some embodiments, the nucleic acid molecules used in the methods described herein are DNA molecules. In some embodiments, the nucleic acid molecules used in the methods described herein are circular, for example plasmids. Alternatively, additional endonuclease sites can be used linearize the nucleic acid molecules of the disclosure. Exemplary endonuclease sites include, but are not limited to restriction endonucleases, as well as the CRISPR/Cas endonucleases, ZFNs and TALENs described herein. The skilled artisan will be able to incorporate suitable endonuclease sites into the nucleic acid molecules, for example adjacent to or near to either or both homology arms of the nucleic acid molecule. The skilled artisan will be able to incorporate suitable CRE recombinase sites into the nucleic acid molecules.

In some embodiments, the target sequence is deleted by the insertion of the template sequence, and the template and target chromosomes are cut on either side of the template and target sequences by CRISPR/Cas ribonucleoproteins. In some embodiments, (a) the target chromosome comprises, from 5′ to 3′, the sequence of the 5′ homology arm of the first nucleic acid molecule, a first sgRNA target sequence, the target sequence, a second sgRNA target sequence, and the sequence of 3′ homology arm of the second nucleic acid molecule and (b) the template chromosome comprises, from 5′ to 3′, a third sgRNA target sequence, the sequence of the 3′ homology arm of the first nucleic acid molecule, the template sequence, the sequence of the 5′ homology arm of the second nucleic acid molecule, and a fourth sgRNA target sequence. In some embodiments, the first, second, third and fourth sgRNAs comprise different targeting sequences. For example the first sgRNA comprises a targeting sequence specific to the first sgRNA target sequence on the target chromosome, the second sgRNA comprises a targeting sequence specific to the second sgRNA target sequence on the target chromosome, the third sgRNA comprises a targeting sequence specific to the third sgRNA target sequence on the template chromosome, and the fourth sgRNA comprises a targeting sequence specific to the fourth sgRNA target sequence on the target chromosome. Alternatively, one or more of the sgRNA target sequences, and corresponding sgRNA targeting sequences, may be the same sequence.

In some embodiments, inserting the template sequence comprises little or no deletion of a sequence of the target sequence. The person of ordinary skill in the art will appreciate that in many mechanisms of double strand break repair involve resection of the ends of the break and will thus generate deletions around the endonuclease sites described herein. For example, deletions of about 5 bp, 10 bp, 15 bp, 20 bp, 25 bp, 30 bp, 35 bp, 40 bp, 45 bp, or 50 bp around the target location, or the endonuclease sites flanking the target sequence, may be generated by the methods described herein.

In some embodiments, for example those embodiments where little or no target sequence is deleted by the methods described herein, (a) the target chromosome comprises, from 5′ to 3′, the sequence of the 5′ homology arm of the first nucleic acid molecule, a first sgRNA target sequence, and the sequence of 3′ homology arm of the second nucleic acid molecule; and (b) the template chromosome comprises, from 5′ to 3′, a second sgRNA target sequence, the sequence of the 3′ homology arm of the first nucleic acid molecule, the template sequence, the sequence of the 5′ homology arm of the second nucleic acid molecule, and a third sgRNA target sequence. In some embodiments, the first, second, and third sgRNAs comprise different targeting sequences. For example the first sgRNA comprises a targeting sequence specific to the first sgRNA target sequence on the target chromosome, the second sgRNA comprises a targeting sequence specific to the second sgRN target sequence on the target chromosome, and the third sgRNA comprises a targeting sequence specific to the third sgRNA target sequence on the template chromosome.

In some embodiments, inserting the template sequence disrupts one or more functions of the target sequence. For example, insertion of the template sequence into coding sequence ofa gene can prevent expression of a proper gene product through the creation of a premature stop codon, a mutation in the protein coding sequence, abnormal splice products and the like. Similarly, insertion of the template sequence into a regulatory sequence of a gene, such as an enhancer or promoter, can prevent the gene from being expressed.

In some embodiments, the methods of the disclosure comprise deleting the first and/or second marker following insertion of the target sequence. Markers can be deleted by any suitable methods known in the art. For example, cells comprising the engineered chromosome can be contacted with a CRISPR/Cas ribonucleoprotein comprising a gNA targeting sequence specific for the sequence encoding the marker, thereby inducing deletion of all or part of the marker sequence.

The methods of the disclosure can be used to generate chromosomal rearrangements, such as inversions and translocations. Many chromosomal rearrangements play a role in human diseases or disorder, such as cancer. Re-creating such rearrangements in a model organism, such as a mouse, can facilitate study of these diseases or disorders. Chromosomal aberrations implicated will be known to persons of skill in the art, and are described in the Mitelman database, available at mitelmandatabase.isb-cgc.org/. Further information about chromosomal aberrations implicated in human diseases is also available at rarediseases.info.nih.gov/diseases/diseases-by-category/36/chromosome-disorders.

Accordingly, the disclosure provides methods of generating a chromosomal rearrangement comprising: (a) providing a cell comprising a target chromosome comprising a target location and a template chromosome comprising a template sequence; (b) contacting the cell with a nucleic acid molecule comprising from 5′ to 3′, a 5′ homology arm comprising a nucleotide sequence upstream of the 5′ end of the target location, a marker, and a 3′ homology arm comprising a nucleotide sequence upstream of the 5′ end of the template sequence; (c) generating double strand breaks at the target location, and at the 5′ end of the template sequence, whereby the marker is inserted in the target chromosome 3′ of the sequence of the 5′ homology arm, followed by the template sequence, thereby generating a chromosomal rearrangement; and (c) selecting a cell or cells expressing the marker. Alternatively, the methods comprise (a) providing a cell comprising a target chromosome comprising a target location and a template chromosome comprising a template sequence; (b) contacting the cell with a nucleic acid molecule comprising from 5′ to 3′, a 5′ homology arm comprising a nucleotide sequence downstream of the 3′ end of the template sequence, a marker, and a 3′ homology arm comprising a nucleotide sequence downstream of the 3′ end of the target sequence; (c) generating double strand breaks at the target location, and at the 3′ end of the template sequence, whereby the marker is inserted in the target chromosome 3′ of the sequence of the 5′ homology arm, followed by the template sequence, thereby generating a chromosomal rearrangement; and (c) selecting a cell or cells expressing the marker. In some embodiments, generating the double stranded breaks comprises contacting the cell with a CRISPR/Cas endonuclease, at least a first gNA comprising a targeting sequence specific to the target location, such that the CRISPR/Cas endonuclease cleaves the target location, and a second gNA comprising a targeting sequence specific to the 5′ end of the template sequence. In some embodiments, generating the double stranded breaks comprises contacting the cell with a CRISPR/Cas endonuclease, at least a first gNA comprising a targeting sequence specific to the target location, such that the CRISPR/Cas endonuclease cleaves the target location, and a second gNA comprising a targeting sequence specific to the 3′ end of the template sequence. In some embodiments, the nucleic acid molecule comprises DNA. In some embodiments, the nucleic acid molecule comprises a plasmid.

Suitable methods known in the art may be used to generate double strand breaks in the target and template chromosomes. This can be accomplished, inter alia, through the selection of homology arm sequences for the nucleic acid molecules (e.g., plasmids) used guide the HDR-mediated chromosomal rearrangement that overlap or comprise the endonuclease sites on the target and template chromosomes. In some embodiments, generating the double strand breaks at (c) comprises using a CRISPR/Cas endonuclease and one or more guide nucleic acids (gNAs), one or more zinc finger nucleases, one or more Transcription Activator-Like Effector Nucleases (TALENs), or one or more CRE recombinase to induce the double strand breaks. For example, Cre recombinase induced an inversion of the chromosomal region between the two LoxP sites, whereby the template sequence and the first and second markers are inserted into the target chromosome. In some embodiments, the CRISPR/Cas endonuclease comprises CasI, CasIB, Cas2, Cas3, Cas4, Cas5, Cas6, Cas7, Cas8, Cas9, Cas10, CasX, CasY, Cas12a (Cpf1), Cas13a, Csy1, Csy2, Csy3, Cse1, Cse2, CscI, Csc2, Csa5, Csn2, Csm2, Csm3, Csm4, Csm5, Csm6, Cmr1, Cmr3, Cmr4, Cmr5, Cmr6, CsbI, Csb2, Csb3, Csx17, Csx14, Csx10, Csx16, CsaX, Csx3, Csx1, Csx15, Csf1, Csf2, Csf3, Csf4, Cms1, C2c1, C2c2, or C23, or a homolog, ortholog, or modified version thereof. In some embodiments, the CRISPR/Cas endonuclease comprises Cas9, Cas12a (Cpf1), Cas13a, CasX, CasY, C2c1, or C2c3. In some embodiments, the CRISPR/Cas endonuclease comprises Cas9. In some embodiments, the gNA comprises a single guide RNA (sgRNA).

Any suitable methods known in the art may be used to contact the cell with the endonucleases described herein. For example, nucleic acid molecules (e.g., plasmids or the like) comprising the endonucleases, and sequences encoding gNAs, for CRISPR/Cas endonucleases, may be used to transfect the cells. Alternatively, endonucleases, or nucleic acid molecules encoding endonucleases, may be introduced into the cells by electroporation, lipofection, transduction, and the like.

The cells used to carry out the methods described herein may be any suitable cells known in the art. In some embodiment the cells comprise embryonic stem (ES) cells. In some embodiment the cells comprise embryonic hybrid stem (EHS) stem cells. EHS cells can be created by fusing ES cells from two different species, for example human and mouse, human and rat, or mouse and monkey. All methods of fusion known in the art are envisaged as within the scope of the instant disclosure, including, but not limited to, electrofusion, viral-induced fusion and chemically induced fusion. In some embodiments, the methods comprise fusing a human EH cell to an EH cell selected from the group consisting of mouse, rat, rabbit, guinea pig, hamster, sheep, goat, donkey, cow, horse, camel, chicken and monkey. In some embodiments, the method comprises fusing EH cells from any two different species selected from the group consisting of mouse, rat, rabbit, guinea pig, hamster, sheep, goat, donkey, cow, horse, camel, chicken and monkey.

In some embodiment the cells comprise zygotes. As used herein, the term “zygote” refers to a eukaryotic cell formed by a fertilization event between two gametes, e.g., an egg and a sperm from a mammal. Zygotes at the single cell, 2 cell, 4 cell, 8 cell or further stages may be suitable for the methods described herein.

Following generating the engineered chromosomes as described herein, any suitable methods may be used to recover the engineered chromosomes. In some embodiments, recovering the engineered chromosomes of the disclosure comprises micro-cell mediated chromosome transfer (MMCT). Recovered chromosomes transferred to any suitable cell type for downstream applications by fusion of micronucleated cells comprising the engineered chromosome to a target cell, such as an ES cell. These methods are described in more detail below.

### Template Chromosome

The disclosure provides template chromosomes, comprising template sequences, for use in the methods described herein.

As used herein, a “template chromosome” refers to a chromosome containing a “template sequence.” The template sequence refers to the sequence to be introduced into the target chromosome, or target location, using the methods of the disclosure.

The template chromosome can be isolated or derived from any suitable source. In some embodiments, the template chromosome is from a eukaryote. In some embodiments, the eukaryote is a vertebrate, such as a bird, reptile or mammal. In some embodiments, the template chromosome is from a mouse, rat, rabbit, guinea pig, hamster, sheep, goat, donkey, cow, horse, camel, monkey or chicken. In some embodiments, the template chromosome is from a human.

In some embodiments, the template chromosome is an exogenous chromosome, and the template sequence is an exogenous sequence. For example, the target chromosome is a mouse chromosome, and the template chromosome and corresponding template sequence are from a non-mouse species, such as a human.

In some embodiments, the template chromosome is an endogenous chromosome, and the template sequence is an endogenous sequence. For example, the template chromosome is a mouse chromosome, and the target chromosome is a second, different, mouse chromosome.

In some embodiments, the template chromosome is an artificial chromosome.

In some embodiments, the template chromosome is a naturally occurring chromosome.

In some embodiments, the template chromosome comprises one or modifications to a naturally occurring chromosome. Modifications include, inter alia, insertions of sequences, deletions, and rearrangements. Examples of sequences inserted in a template chromosome include, inter alia, markers, promoters, cDNA sequences, non-coding and the like.

In some embodiments, the template chromosome comprises an endonuclease site located 5′ of the template sequence. In some embodiments, the template chromosome comprises an endonuclease site located 3′ of the template sequence. In some embodiments, the endonuclease site is located immediately adjacent to the template sequence. In some embodiments, the endonuclease site is located near the template sequence

In some embodiments, the template chromosome comprises an endonuclease site on either side of the template sequence. For example, the template chromosome comprises a first endonuclease site located 5′ of the template sequence and a second endonuclease site located 3′ of the template sequence. In some embodiments, both the first and second endonuclease sites are recognized and cleaved by the same endonuclease. For example, both the first and second endonuclease sites comprise the same DNA sequence, that is recognized by the same endonuclease. In some embodiments, the first endonuclease site is cleaved by a first endonuclease, and the second endonuclease site is cleaved by a second endonuclease. For example, the first and second endonuclease sites comprise different DNA sequences that recognized by two different zinc finger nucleases (ZFNs), or two different CRISPR/Cas target sequences that are recognized by CRISPR/Cas ribonucleoprotein complexes comprising guide nucleic acids (gNAs) comprising different targeting sequences. In some embodiments, the first and/or second endonuclease site is located immediately adjacent to the template sequence. In some embodiments, the first and/or second endonuclease site is located near the template sequence.

A sequence that is within 5 basepairs (bp), within 10 bp, within 15 bp, within 20 bp, within 30 bp, within 40 bp, within 50 bp, within 70 bp, within 80 bp, within 90 bp, within 100 bp, within 120 bp, within 140 bp, within 160 bp, within 180 bp, within 200 bp, within 250 bp, within 300 bp, within 400 bp or within 500 bp of the template sequence can be considered to be near the template sequence.

In some embodiments, the template chromosome comprises one or more sequences of homology arms of nucleic acid molecules used to facilitate homology directed repair. In some embodiments, the template chromosome comprises a sequence of a homology arm located at or near the 5′ end of the template sequence. In some embodiments, the homology arm is located upstream, i.e. 5′ of, the template sequence. In some embodiments, the template chromosome comprises, from 5′ to 3′, an endonuclease site, a homology arm sequence, and the template sequence. In some embodiments, the template chromosome comprises a sequence ofa homology arm located at or near the 3′ end of the template sequence. In some embodiments, the homology arm is located downstream, i.e. 3′ of, the template sequence. In some embodiments, the template chromosome comprises, from 5′ to 3′, the template sequence, the homology arn sequence, and an endonuclease site. In some embodiments, the homology arm sequence is located between endonuclease site and the template sequence.

In some embodiments, the template chromosome comprises a first homology arm sequence located at or near the 5′ of the template sequence, and a second homology arm sequence located at or near the 3′ of the template sequence. I.e., the template chromosome comprises homology arms upstream and downstream of the template sequence. In some embodiments, the first homology arm is a 3′ homology arm of a first nucleic acid molecule comprising from 5′ to 3′, a 5′ homology arm comprising a nucleotide sequence upstream of the 5′ end of the target sequence, a sequence of at least a first marker, and the first homology arm sequence. In some embodiments, the second homology arm is a 5′ homology arm of a second nucleic acid molecule comprising from 5′ to 3′, the second homology arm sequence, a sequence of at least a second marker, and a 3′ homology arm comprising a nucleotide sequence downstream of the 3′ end of the target sequence. In some embodiments, the template chromosome comprises, from 5′ to 3′, the first endonuclease site, the first homology arm sequence, the template sequence, the second homology arm sequence, and the second endonuclease site.

In some embodiments, the first and/or second homology arm sequence is located immediately adjacent to the first and/or second endonuclease site. In some embodiments, the first homology arm sequence is located immediately adjacent to the first endonuclease site, and the second homology arm sequence is located immediately adjacent to the second endonuclease site, wherein the first homology arm is between the first endonuclease site and the template sequence, and the second homology arm is between the template sequence and the second template sequence. In some embodiments, the first homology arm is between the first endonuclease site and the template sequence, and the second homology arm is between the template sequence and the second template sequence.

In some embodiments, the first and/or second homology arm sequence is located near the template sequence. An homology arm that is within 0 bp, 5 basepairs (bp), within 10 bp, within 15 bp, within 20 bp, within 30 bp, within 40 bp, within 50 bp, within 70 bp, within 80 bp, within 90 bp, within 100 bp, within 120 bp, within 140 bp, within 160 bp, within 180 bp, within 200 bp or within 250 bp of the template sequence can be considered to be near the template sequence.

In some embodiments, the template chromosome comprises, from 5′ to 3′, the first endonuclease site, the first homology arm, the template sequence, the second homology arm, and the second endonuclease site.

In some embodiments, the first and/or second homology sequences of the template chromosome are between about 20 and 2,000 bp, between about 50 and 1,500 bp, between about 100 and 1,400 bp, between about 150 and 1,300 bp, between about 200 and 1,200 bp, between about 300 and 1,100 bp, between about 400 and 1,000 bp, or between about 500 and 900 bp, or between about 600 bp and 800 bp in length. In some embodiments, the homology sequences of the template chromosome are between about 400 and 1,500 bp in length. In some embodiments, the homology sequences of the template chromosome are between about 500 and 1,300 bp in length. In some embodiments, the homology sequences of the template chromosome are between about 600 and 1,000 bp in length.

### Template Sequence

The template chromosome comprises the template sequence, and serves as the source of the template sequence in the engineered chromosomes and the methods described herein. The template sequence can be located at any suitable location on the template chromosome. For example, and without wishing to be bound by theory, a template sequence may be located in a region of the template chromosome characterized by euchromatin.

The template sequence can be isolated or derived from any suitable source. In some embodiments, the template sequence comprises an endogenous sequence, for example a sequence endogenous to the template chromosome, or a sequence endogenous to the species that gave rise to the target chromosome. In some embodiments, the template sequence is an exogenous sequence. For example, the template sequence is from a sequence exogenous to the species that gave rise to the target chromosome. In some embodiments, the template sequence comprises a naturally occurring sequence. In some embodiments, the template sequence comprises one or modifications to a naturally occurring sequence. Modifications include, inter alia, insertions of sequences such as artificial sequences or markers, deletions, and rearrangements. In some embodiments, the template sequence comprises an artificial sequence. In some embodiments, the template sequence comprises both naturally occurring and artificial sequences. Exemplary artificial sequences include, inter alia, markers, cDNA sequences, promoters, and recombinant sequences. Exemplary markers include, but are not limited to, the selectable markers disclosed in Table 3 below, as well as detectable markers such as green fluorescent protein (GFP), mCherry and the like.

In some embodiments, the template sequence is from a eukaryote. In some embodiments, the eukaryote is a vertebrate, such as a bird, reptile or mammal. In some embodiments, the template sequence comprises a mouse, rat, rabbit, guinea pig, hamster, sheep, goat, donkey, cow, horse, camel, monkey or chicken sequence. In some embodiments, the template sequence comprises a human sequence.

In some embodiments, the template sequence is at least 25 KB, at least 50 KB, at least 100 KB, at least 200 KB, at least 400 KB, at least 500 KB, at least 600 KB, at least 700 KB, at least 800 KB, at least 900 KB, at least 1 MB, at least 2 MB, at least 3 MB, at least 4 MB, at least 5 MB, at least 6 MB, at least 7 MB, at least 8 MB, at least 9 MB, at least 10 MB, at least 15 MB, at least 20 MB, at least 25 MB, at least 30 MB, at least 40 MB, at least 50 MB, at least 60 MB, at least 70 MB, at least 80 MB, at least 90 MB, at least 100 MB, at least 120 MB, at least 140 MB, at least 160 MB, at least 180 MB, at least 200 MB, at least 220 MB, or at least 250 MB in length. In some embodiments, the template sequence is at least 50 KB, at least 100 KB, at least 200 KB, at least 500 KB, at least 700 KB, at least 1 MB, at least 2 MB, at least 3 MB, at least 4 MB, at least 5 MB, at least 6 MB, at least 71 MB, at least 8 MB, at least 9 MB, at least 10 MB, at least 20 MB, at least 30 MB, at least 40 MB, or at least 50 M1B in length. In some embodiments, the template sequence is at least 1 MB in length. In some embodiments, the template sequence is at least 2 MB in length. In some embodiments, the template sequence is at least 3 MB in length. In some embodiments, the template sequence is at least 4 MB in length. In some embodiments, the template sequence is at least 5 MB in length. In some embodiments, the template sequence is at least 10 MB in length. In some embodiments, the template sequence is at least 20 MB in length.

In some embodiments, the template sequence is between 50 KB and 250 MB, 50 KB and 100 MB, 50 KB and 50 MB., 50 KB and 20 MB., 50 KB and 10 MB, 50 KB and 5 MB, 50 KB and 3 MB, 50 KB and 2 MB, 50 KB and 1 MB, 100 KB and 200 MB, 100 KB and 100 MB, 100 KB and 50 MB, 100 KB and 20 MB, 100 KB and 10 MB, 100 KB and 5 MB, 100 KB and 3 MB, 100 KB and 2 MB, 100 KB and 1 MB, 100 KB and 500 KB, 200 KB and 100 MB, 200 KB and 50 MB, 200 KB and 20 MB, 200 KB and 10 MB, 200 KB and 5 MB, 200 KB and 3 MB, 200 KB and 2 MB, 200 KB and 1 MB, 200 KB and 500 KB, 500 KB and 100 113, 500 KB and 50 MB, 500 KB and 20 MB, 500 KB and 10 MB, 500 KB and 5 MB, 500 KB and 3 MB, 500 KB and 2 MB, 500 KB and 1 MB, 1 MB and 100 MB, 1 MB and 50 MB, 1 MB and 20 MB, 1 MB and 10 MB, 1 MB and 5 MB, 1 MB and 3 MB, 1 MB and 2 MB, 3 MB and 100 MB, 3 MB and 50 MB, 3 MB and 20 MB, 3 MB and 10 MB, 3 MB and 5 MB, 5 MB and 100 MB, 5 MB and 50 MB, 5 MB and 20 MB, 5 MB and 10 MB, 10 MB and 100 MB, 10 MB and 50 MB, or 10 MB and 20 MB, in length. In some embodiments, the template sequence is between 50 KB and 250 MB in length. In some embodiments, the template sequence is between 500 KB and 200 MB in length. In some embodiments, the template sequence is between 200 KB and 50 MB, between 1 MB and 20 MB, between 1 MB and 10 MB, between 1 MB and 5 MB, between 1 MB and 3 MB, between 3 MB and 20 MB, between 3 MB and 10 MB, between 3 MB and 7 MB, or between 3 MB and 5 MB in length. In some embodiments, the template sequence is between 1 MB and 10 MB in length. In some embodiments, the template sequence is between 1 MB and 5 MB in length. In some embodiments, the template sequence is between 3 MB and 5 MB in length.

In some embodiments, the template sequence comprises sequences of one or more genes. In some embodiments, the template sequence comprises sequences of multiple genes. In some embodiments, the template sequence comprises the sequence of at least 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900, 1000, 1500 or 2000 genes.

In some embodiments, the template sequence comprises a human sequence, such as a sequence of one or more human genes. In some embodiments, the template sequence comprises a subsequence of a human gene. In some embodiments, the template sequence comprises a subsequence of a human gene and an artificial sequence, such as a marker or a fusion protein. In some embodiments, the template sequence comprises sequences of one or more human genes and an artificial sequence.

In some embodiments, the template sequence comprises a sequence of a human gene. All human genes are envisaged within the scope of the instant disclosure. Without wishing to be bound by theory, transfer of human genes involved in disease pathogenesis, or that are potential therapeutic targets, to a model organism such as a mouse can facilitate research into the disease and development of suitable therapies.

Exemplary genes for inclusion in the template sequence include, but are not limited to, immunoglobulin genes, T cell receptor (TCR) genes, immune checkpoint genes, cytokines, chernokines, receptors, transcription factors, cytoskeletalgenes, cell cycle check genes, oncogenes, and genes involved in development, immunology or neurobiology. Exemplary immune checkpoint genes include BTLA, CTLA-4, TIM-3, PD-1 and PD-L1. Exemplary cytokines include interleukins (CTNF, IL-16, IL-1B, IL-6, IL-12, IL-17F, IL-2, IL-3, IL-9, IL-12B, IL18BP, TL-21, 1L33, Leptin, IL-13, IL1A, IL-23, IL-4), interferons (IFNA10, IFN-alpha7, IFNa4Fc, IFN beta, IFNA alpha 4, IFN gamma, IFNA alpha 5, IFN omega), and tumor necrosis factors (TNFs, e.g. BAFF, TNF beta, CD30 ligand, TNF alpha, CD40 ligand, TNFSF10, CD27 ligand). Exemplary chemokines include CXC, CC CX3C′ and C family chemokines. Exemplary receptors include G protein coupled receptors, ligand-gated ion channels (ionotropic receptors), kinase-linked receptors and related receptors, and nuclear receptors. Exemplary transcription factors include, but are not limited to, helix-turn-helix transcription factors (e.g. Oct-1), helix-loop-helix transcription factors (e.g. E2A), zinc finger transcription factors (e.g. glucocorticoid receptors, GATA proteins), basic protein-leucine zipper transcription factors (e.g. cyclic AMP response element-binding factor (CRE), and activator protein-1 (API)), and β-sheet motif transcription factors (e.g. nuclear factor-κB (NF-κB)). Exemplary cell cycle regulator genes include, but are net limited to cyclins, cyclin dependent kinases, and cell cycle checkpoint gene.

In some embodiments, the template sequence comprises an oncogene or tumor suppressor gene. Exemplary oncogenes and tumor suppressor genes suitable for inclusion in the template sequence are presented in Table 1 below

TABLE 1
Oncogenes and tumor suppressors

| Symbol | Gene Name |
|---|---|
| ABL1 | c-abl oncogene 1, non-receptor tyrosine kinase |
| ABL2 | ABL proto-oncogene 2, non-receptor tyrosine |
| AKAP13 | A-kinase anchoring protein 13 |
| AKT2 | AKT serine/threonine kinase 2 |
| APC | adenomatous polyposis coli |
| ARAF | A-Raf proto-oncogene, serine/threonine kinase |
| ATM | ataxia telangiectasia mutated |
| ATR | ataxia telangiectasia and Rad3 related |
| AXL | AXL receptor tyrosine kinase |
| BAX | BCL2-associated X protein |
| BCL2 | B-cell CLL/lymphoma 2 |
| BCL3 | B-cell CLL/lymphoma 3 |
| BCL6 | B-cell CLL/lymphoma 6 |
| BCR | breakpoint cluster region |
| BIN1 | bridging integrator 1 |
| BRAF | B-Raf proto-oncogene, serine/threonine |
| BRCA1 | BRCA1 DNA repair associated |
| BRCA2 | BRCA2 DNA repair associated |
| CCDC6 | coiled-coil domain containing 6 |
| CCNA2 | cyclin A2 |
| CCNE1 | cyclin E1 |
| CD82 | CD82 molecule |
| CDC25A | cell division cycle 25A |
| CDH1 | cadherin 1, type 1, E-cadherin |
| CDK4 | cyclin-dependent kinase 4 |
| CDK6 | cyclin-dependent kinase 6 |
| CDKN1A | cyclin-dependent kinase inhibitor 1A (p21, Cip1) |
| CDKN1C | cyclin-dependent kinase inhibitor 1C (p57, Kip2) |
| CDKN2A | cyclin-dependent kinase inhibitor 2A |
| CDKN2B | cyclin-dependent kinase inhibitor 2B |
| CDKN2C | cyclin-dependent kinase inhibitor 2C |
| CEACAM7 | carcinoembryonic antigen-related cell adhesion molecule 7 |
| COL4A3 | collagen, type IV, alpha 3 |
| CSF1 | colony stimulating factor 1 |
| CSNK2A1 | casein kinase 2, alpha 1 polypeptide |
| CTNNB1 | catenin (cadherin-associated protein), beta 1 |
| CXCL1 | chemokine (C-X-C motif) ligand 1 |
| CXCL2 | chemokine (C-X-C motif) ligand 2 |
| CXCL3 | chemokine (C-X-C motif) ligand 3 |
| CYP19A1 | cytochrome P450, family 19, subfamily A, polypeptide 1 |
| DCC | deleted in colorectal carcinoma |
| DDX6 | DEAD (Asp-Glu-Ala-Asp) box polypeptide 6 |
| E2F1 | E2F transcription factor 1 |
| EGFR | epidermal growth factor receptor |
| EIF1AX | eukaryotic translation initiation factor 1A, X-linked |
| EIF2AK2 | eukaryotic translation initiation factor 2-alpha kinase 2 |
| EIF4E | eukaryotic translation initiation factor 4E |
| ELK1 | ELK1, member of ETS oncogene family |
| ELL | elongation factor RNA polymerase II |
| EMP1 | epithelial membrane protein 1 |
| EPHA1 | EPH receptor A1 |
| EPOR | erythropoietin receptor |
| ERBB2 | erb-b2 receptor tyrosine kinase 2 |
| ERBB3 | erb-b2 receptor tyrosine kinase 3 |
| ERBB4 | erb-b2 receptor tyrosine kinase 4 |
| ETS2 | ETS proto-oncogene 2, transcription facto |
| ETV3 | ets variant 3 |
| ETV6 | ets variant 6 |
| EWSR1 | Ewing sarcoma breakpoint region 1 |
| FABP3 | fatty acid binding protein 3 |
| FAT2 | FAT tumor suppressor homolog 2 |
| FES | FES proto-oncogene, tyrosine kinase |
| FGF3 | fibroblast growth factor 3 |
| FGF4 | fibroblast growth factor 4 |
| FGF5 | fibroblast growth factor 5 |
| FGF6 | fibroblast growth factor 6 |
| FGF8 | fibroblast growth factor 8 |
| FHIT | fragile histidine triad gene |
| FLT1 | fms-related tyrosine kinase 1 |
| FOSL1 | FOS-like antigen 1 |
| FOSL2 | FOS-like antigen 2 |
| FYN | FYN proto-oncogene, Src family tyrosine kinase |
| GSTM1 | glutathione S-transferase mu 1 |
| GSTT1 | glutathione S-transferase theta 1 |
| HIC1 | hypermethylated in cancer 1 |
| HOXB8 | homeobox B8 |
| IGF2R | insulin-like growth factor 2 receptor |
| ING3 | inhibitor of growth family, member 3 |
| JUN | jun proto-oncogene |
| LCK | lymphocyte-specific protein tyrosine kinase |
| LMO1 | LIM domain only 1 (rhombotin 1) |
| LMO2 | LIM domain only 2 (rhombotin-like 1) |
| LTA | lymphotoxin alpha (TNF superfamily, member 1) |
| MAFG | MAF bZIP transcription factor G |
| MAS1 | MAS1 oncogene |
| MCC | mutated in colorectal cancers |
| MDM2 | MDM2 proto-oncogene |
| MEN1 | multiple endocrine neoplasia I |
| MERTK | c-mer proto-oncogene tyrosine kinase |
| MET | met proto-oncogene |
| MFHAS1 | malignant fibrous histiocytoma amplified sequence 1 |
| MLH1 | mutL homolog 1 |
| MLL | myeloid/lymphoid or mixed-lineage leukemia |
| MOS | MOS proto-oncogene, serine/threonine kinase |
| MPL | MPL proto-oncogene, thrombopoietin receptor |
| MSH2 | mutS homolog 2 |
| MXI1 | MAX interactor 1 |
| MYCL1 | MYCL proto-oncogene, bHLH transcription factor |
| NBL1 | NBL1, DAN family BMP antagonist |
| NCK1 | NCK adaptor protein 1 |
| NF2 | neurofibromin 2 |
| NOTCH4 | notch 4 |
| NOV | nephroblastoma overexpressed gene |
| NTRK1 | neurotrophic tyrosine kinase, receptor, type 1 |
| PBX2 | pre-B-cell leukemia homeobox 2 |
| PDGFB | platelet-derived growth factor beta polypeptide |
| PDGFRL | platelet-derived growth factor receptor-like |
| PLA2G2A | phospholipase A2, group IIA |
| PML | promyelocytic leukemia |
| PRDM2 | PR domain containing 2, with ZNF domain |
| PRKCDBP | protein kinase C, delta binding protein |
| PRLR | prolactin receptor |
| PTCH1 | patched 1 |
| PTEN | phosphatase and tensin homolog |
| PVT1 | Pvt1 oncogene (non-protein coding) |
| RAB8A | RAB8A, member RAS oncogene family |
| RAF1 | Raf-1 proto-oncogene, serine/threonine kinase |
| RB1 | retinoblastoma 1 |
| RB1CC1 | RB1-inducible coiled-coil 1 |
| RELA | v-rel reticuloendotheliosis viral oncogene homolog A |
| RET | ret proto-oncogene |
| RHOA | ras homolog gene family, member A |
| RHOB | ras homolog gene family, member B |
| RHOC | ras homolog gene family, member C |
| ROS1 | c-ros oncogene 1, receptor tyrosine kinase |
| SHC1 | SHC adaptor protein 1 |
| SKI | v-ski sarcoma viral oncogene homolog |
| SKIL | SKI-like oncogene |
| SKP2 | S-phase kinase-associated protein 2 (p45) |
| SMAD2 | SMAD family member 2 |
| SMAD4 | SMAD family member 4 |
| SMARCBI | SWI/SNF related, matrix associated, actin dependent regulator of |
| | chromatin, subfamily b, member 1 |
| SRC | SRC proto-oncogene, non-receptor tyrosine kinase |
| TP53 | tumor protein p53 |
| WT1 | WT1 transcription factor |
| WNT2 | Wnt family member 2 |
| WNT10B | Wnt family member 10B |
| WNT5A | Wnt family member 5A |
| WNT3 | Wnt family member 3 |
| WNT1 | Wnt family member 1 |
| VHL | von Hippel-Lindau tumor suppressor |
| USP4 | ubiquitin specific peptidase 4 |
| TNF | tumor necrosis factor |
| TERT | telomerase reverse transcriptase |
| TGFBR2 | transforming growth factor beta receptor 2 |
| TGFBR1 | transforming growth factor beta receptor 1 |
| TAL1 | TAL bHLH transcription factor 1, erythroid differentiation factor |
| TP73 | tumor protein p73 |
| TSG101 | tumor susceptibility 101 |
| EIF3E | eukaryotic translation initiation factor 3, subunit E |
| FOXG1 | forkhead box G1 |

In some embodiments, the template sequence comprises a sequence of a human gene associated with a genetic disease or disorder. In some embodiments, the template sequence comprises a sequence of a human chromosomal region associated with a genetic disease or disorder. Non-limiting examples of genes, and chromosomal regions, that are associated with diseases or disorders are presented in Table 2 below.

TABLE 2
Genetic diseases or disorders, and associated genes or genomic regions

| Disease or Disorder | Gene(s) or Chromosomal Region |
|---|---|
| Aceruloplasminemia | ceruloplasmin (CP) |
| Acheiropodia | limb development membrane protein 1 (LMBR1) |
| Achondrogenesis type II | collagen type II alpha 1 chain (COL2A1) |
| achondroplasia | fibroblast growth factor receptor 3 (FGFR3) |
| Acute intermittent porphyria | hydroxymethylbilane synthase (HMBS) |
| Adrenoleukodystrophy | ATP binding cassette subfamily D member 1 (ABCD1) |
| Alagille syndrome | JAG1 - jagged canonical Notch ligand 1 (JAG1), notch |
| | receptor 2 (NOTCH2) |
| Alexander disease | glial fibrillary acidic protein (GFAP) |
| Alport syndrome | collagen type IV alpha 3 chain (COL4A3), COL4A4, |
| | and COL4A5 |
| Amyotrophic lateral sclerosis | C9orf72-SMCR8 complex subunit (C9orf72), superoxide |
| | dismutase 1 (SOD1), FUS RNA binding protein |
| | (FUS), TAR DNA binding protein (TARDBP), coiled-coil- |
| | helix-coiled-coil-helix domain containing 10 |
| | (CHCHD10), microtubule associated protein tau (MAPT) |
| Alström syndrome | ALMS1 centrosome and basal body associated (ALMS1) |
| Aminolevulinic acid | aminolevulinate dehydratase (ALAD) |
| dehydratase deficiency | |
| porphyria | |
| Angelman syndrome | ubiquitin protein ligase E3A (UBE3A) |
| Apert syndrome | fibroblast growth factor receptor 2 (FGFR2) |
| Ataxia telangiectasia | ATM serine/threonine kinase (ATM) |
| Axenfeld syndrome | paired like homeodomain 2 (PITX2), forkhead box O1 |
| | (FOXO1A), forkhead box C1 (FOXC1), paired box 6 |
| | (PAX6) |
| biotinidase deficiency | biotinidase (BTD) |
| Brody myopathy | ATPase sarcoplasmic/endoplasmic reticulum Ca2+ |
| | transporting 1 (ATP2A1) |
| Brunner syndrome | monoamine oxidase A (MAOA) |
| CADASIL syndrome | notch receptor 3 (NOTCH3) |
| Campomelic dysplasia | X 17q24.3-q25.1 |
| Carpenter Syndrome | RAB23, member RAS oncogene family (RAB23) |
| CDKL5 deficiency disorder | cyclin dependent kinase like 5 (CDKL5) |
| Cystic fibrosis | CF transmembrane conductance regulator (CFTR) |
| Charcot-Marie-Tooth disease | peripheral myelin protein 22 (PMP22), mitofusin 2 |
| | (MFN2) |
| Chondrodysplasia, Grebe type | growth differentiation factor 5 (GDF5) |
| Coffin-Lowry syndrome | ribosomal protein S6 kinase A3 (RPS6KA3) |
| collagenopathy, types II and | collagen type XI alpha 1 chain (COL11A1), collagen type |
| XI | XI alpha 2 chain (COL11A2), collagen type II alpha 1 |
| | chain (COL2A1) |
| Congenital insensitivity to | neurotrophic receptor tyrosine kinase 1 (NTRK1) |
| pain with anhidrosis (CIPA) | |
| Cranio-lenticulo-sutural | 14q13-q21 |
| dysplasia | |
| Crouzon syndrome | FGFR2, FGFR3 |
| Dent's disease | chloride voltage-gated channel 5 (CLCN5), OCRL inositol |
| | polyphosphate-5-phosphatase (OCRL) |
| De Grouchy syndrome | 18q |
| Duchenne muscular dystrophy | Dystrophin |
| Dravet syndrome | sodium voltage-gated channel alpha subunit 1 |
| | (SCN1A), SCN2A |
| Fanconi anemia (FA) | FA complementation group A |
| | (FANCA), FANCB, FANCC, FANCD1, FANCD2, FANCE, |
| | FANCF, FANCG, FANCI, FANCJ, FANCL, FANCM, |
| | FANCN, FANCP, FANCS |
| Fabry disease | galactosidase alpha (GLA) |
| Fatal familial insomnia | prion protein (PRNP) |
| Familial adenomatous | APC |
| polyposis | |
| Familial dysautonomia | elongator acetyltransferase complex subunit 1 (IKBKAP) |
| Fragile X syndrome | FMRP translational regulator 1 (FMR1) |
| Friedreich's ataxia | frataxin (FXN) |
| Gaucher disease | glucosylceramidase beta (GBA) |
| Gillespie syndrome | PAX6 |
| Hemochromatosis type 1 | homeostatic iron regulator (HFE) |
| Hemochromatosis type 2A | HFE2A |
| Hemochromatosis type 2B | HFE2B |
| Haemochromatosis type 3 | HFE3 |
| Hemochromatosis type 4 | HFE4 |
| Hemochromatosis type 5 | ferritin heavy chain 1 (FTH1) |
| Hemophilia | coagulation factor VIII (FVIII) |
| Hepatoerythropoietic | uroporphyrinogen decarboxylase (UROD) |
| porphyria | |
| Hereditary coproporphyria | 3q12 |
| Hereditary neuropathy with | PMP22 |
| liability to pressure | |
| palsies (HNPP) | |
| Huntington's disease | Huntingtin (HTT) |
| Hunter syndrome | iduronate 2-sulfatase (IDS) |
| Hurler syndrome | alpha-L-iduronidase (IDUA) |
| Hyperphenylalaninemia | 12q |
| Hypochondrogenesis | COL2A1 |
| Hypochondroplasia | FGFR3 |
| Immunodeficiency- | 20q11.2 |
| centromeric instability-facial | |
| anomalies syndrome (ICF | |
| syndrome) | |
| Incontinentia pigmenti | inhibitor of nuclear factor kappa B kinase regulatory |
| | subunit gamma (IKBKG) |
| Jackson-Weiss syndrome | FGFR2 |
| Kleefstra syndrome | 9q34 |
| Kniest dysplasia | COL2A1 |
| Krabbe disease | galactosylceramidase (GALC) |
| Maroteaux-Lamy syndrome | arylsulfatase B (ARSB) |
| McCune-Albright syndrome | 20 q13.2-13.3 |
| Mediterranean fever, familial | MEFV innate immunity regulator, pyrin (MEFV) |
| Menkes disease | ATPase copper transporting alpha (ATP7A) |
| Microcephaly | assembly factor for spindle microtubules (ASPM) |
| Miller-Dieker syndrome | 17p13.3 |
| Mowat-Wilson syndrome | zinc finger E-box binding homeobox 2 (ZEB2) |
| Muenke syndrome | FGFR3 |
| Multiple endocrine neoplasia | menin 1 (MEN1) |
| type 1 (Wermer's syndrome) | |
| myotonic dystrophy | DM1 protein kinase (DMPK), CCHC-type zinc finger |
| | nucleic acid binding protein (CNBP) |
| Natowicz syndrome | hyaluronidase 1 (HYAL1) |
| Neurofibromatosis type I | 17q11.2 |
| Neurofibromatosis type II | neurofibromin 2 (NF2) |
| Noonan syndrome | protein tyrosine phosphatase non-receptor type 11 |
| | (PTPN11), SOS Ras/Rac guanine nucleotide exchange |
| | factor 1 (SOS1), Raf-1 proto-oncogene, serine/threonine |
| | kinase (RAF1), Ras like without CAAX 1 (RIT1) |
| Omenn syndrome | recombination activating 1 (RAG1), RAG2 |
| Osteogenesis imperfecta | COL1A1, COL1A2, interferon induced transmembrane |
| | protein 5 (IFITM5) |
| Porphyria cutanea tarda (PCT) | uroporphyrinogen decarboxylase (UROD) |
| Pfeiffer syndrome | FGFR1, FGFR2 |
| Phelan-McDermid syndrome | 22q13 |
| Phenylketonuria | phenylalanine hydroxylase (PAH) |
| Pitt-Hopkins syndrome | transcription factor 4 (TCF4) |
| Polycystic kidney disease | PKD1, PKD2 |
| Protein C deficiency | PROC |
| Protein S deficiency | PROS1 |
| Proximal 18q deletion | 18q |
| syndrome | |
| Retinitis pigmentosa | Rhodopsin (RHO) |
| Rett syndrome | methyl-CpG binding protein 2 (MECP2) |
| Sanfilippo syndrome | N-sulfoglucosamine sulfohydrolase (SGSH), N-acetyl- |
| | alpha-glucosaminidase (NAGLU), heparan-alpha- |
| | glucosaminide N-acetyltransferase |
| | (HGSNAT), glucosamine (N-acetyl)-6-sulfatase (GNS) |
| Spondyloepiphyseal dysplasia | COL2A1 |
| congenita (SED) | |
| Sickle cell anemia | 11p15 |
| Sideroblastic anemia | ABCB7, SLC25A38, GLRX5 |
| Sly syndrome | glucuronidase beta (GUSB) |
| Smith-Magenis syndrome | 17p11.2 |
| Snyder-Robinson syndrome | Xp21.3-p22.12 |
| Spinal muscular atrophy | 5q |
| Spinocerebellar ataxia | Ataxin 1 |
| | (ATXN1), ATXN2, ATXN3, ATXN7, ATXN8OS, |
| | ATXN10, pleckstrin homology and RhoGEF domain |
| | containing G4 (PLEKHG4), spectrin beta, non- |
| | erythrocytic (SPTBN2), calcium voltage-gated channel |
| | subunit alpha1 A (CACNA1A), tau tubulin kinase |
| | 2 (TTBK2), protein phosphatase 2 regulatory subunit |
| | Bbeta (PPP2R2B), potassium voltage-gated channel |
| | subfamily C member 3 (KCNC3), protein kinase C gamma |
| | (PRKCG), inositol 1,4,5-trisphosphate receptor type 1 |
| | (ITPR1), TATA-box binding protein (TBP), potassium |
| | voltage-gated channel subfamily D member 3 |
| | (KCND3), FGF14 |
| SSB syndrome (SADDAN) | FGFR3 |
| Stargardt disease (macular | ATP binding cassette subfamily A member 4 (ABCA4) |
| degeneration) | |
| Tay-Sachs disease | hexosaminidase subunit alpha (HEXA) |
| Thanatophoric dysplasia | FGFR3 |
| Treacher Collins syndrome | 5q32-q33.1 |
| Usher syndrome | usherin (USH2A), clarin 1 (CLRN1) |
| Variegate porphyria | protoporphyrinogen oxidase (PPOX) |
| von Willebrand disease | von Willebrand factor (VWF) |
| Weissenbacher-Zweymüller | COL11A2 |
| syndrome | |
| Williams syndrome | 7q11.23 |
| Wilson disease | ATPase copper transporting beta (ATP7B) |
| Woodhouse-Sakati syndrome | C2ORF37 |
| Wolf-Hirschhorn syndrome | 4p16.3 |
| Xeroderma pigmentosum | ERCC excision repair 4, endonuclease catalytic subunit |
| | (ERCC4) |

In some embodiments, the template sequence comprises an immunoglobulin sequence Both surface and secreted immunoglobulins are envisaged as within the scope of the instant disclosure. Immunoglobulins recognize foreign antigens and initiate immune responses. In humans, each immunoglobulin molecule consists of two identical heavy chains, encoded by the IGH locus on chromosome 14, and two identical light chains, which are encoded by the immunoglobulin kappa locus (IGK) on chromosome 2 and the immunoglobulin lambda locus (IGL) on chromosome 22. The IGH locus includes V (variable), D (diversity), J (joining), and C (constant) regions. The V, D and J regions each contain multiple different gene segments, and are referred to collectively herein as the IGH variable regions. During B cell development, a recombination event at the DNA level joins a single D segment with a J segment; the fused D-J exon of this partially rearranged D-J region is then joined to a V segment. The rearranged V-D-J region containing a fused V-D-J exon is then transcribed and fused to the constant region by RNA splicing. This transcript encodes a mu heavy chain. Later in development B cells generate V-D-J-Cmu-Cdelta pre-messenger RNA, which is alternatively spliced to encode either a mu or a delta heavy chain. Mature B cells in the lymph nodes undergo switch recombination, so that the fused V-D-J gene segment is brought in proximity to one of the IGHG, IGHA, or IGHE gene segments and each cell expresses either the gamma, alpha, or epsilon heavy chain. Potential recombination of many different V segments with several J segments provides a wide range of antigen recognition. Additional diversity is attained by junctional diversity, resulting from the random addition of nucleotides by terminal deoxynucleotidyl transferase, and by somatic hypermutation. Each light chain is composed of two tandem immunoglobulin domains, the constant domain (C_{L}) and the variable domain (V_{L}). For the light chain, the V domain is encoded by two separate DNA segments. The first segment is termed a V gene segment because it encodes most of the V domain. The second segment encodes the remainder of the V domain and is termed a joining or J gene segment. Like the heavy chain, the light chain undergoes rearrangement to join a V segment to a J gene segment, and bring the V gene close to a Constant region sequence, which is then separated by only an intron. IGH sequences of any of IGHV, IGHD, IGHJ, IGHG or IGHA, or any combination thereof, are envisaged as within the scope of the template sequences of the disclosure. Light chain sequences of either IGK or IGL, or a combination thereof, are envisaged as within the scope of the template sequences of the disclosure.

In some embodiments, the engineered chromosome comprises a mouse chromosome in which one or more non-coding sequence may have been introduced into said chromosome. For example, one or more non-coding sequence that is capable of regulating antibody generating, maturing and/or diversifying may have been introduced into said chromosome. For example, the one or more non-coding sequence that is capable of regulating antibody diversifying may have been introduced into said chromosome. For example, the one or more non-coding sequence that is capable of regulating antibody class switching may have been introduced into said chromosome. For example, the one or more non-coding sequence within switch region may have been introduced into said chromosome. For example, when the one or more non-coding sequence have been introduced into said chromosome, the class switch recombination, somatic hypermutation and/or activation-induced cytidine deaminase may be regulated. For example, when the one or more non-coding sequence have been introduced into said chromosome, the diversity of repertoire of Ig sequences may be regulated. For example, the variable region of about 2 kb that contains rearranged genes on the heavy, κ light, and λ light chain loci, and/or the switch region of about 4 kb that contains an extensive stretch of G:C rich DNA on the heavy chain locus may have been introduced into said chromosome.

In some embodiments, the template sequence comprises a human IGH sequence. Human IGH spans nucleotide positions 105,586,437 to 106,87⁹,844 chromosome 14 of the GRCh38.p13 assembly of the human genome. The skilled artisan will appreciate that human IGH sequences with 5′ and 3′ boundaries that deviate from those described supra, for example by at least 100 bp, 500 bp, 1,000 bp, 2,000 bp, 5,000 bp, 10,000 bp or more are suitable template sequences.

In some embodiments, the template sequence comprises a human IGH variable region sequence. In some embodiments, the human IGH variable region sequence comprises a sequence encoding human VH, Dt and J1-6 gene segments and intervening non-coding sequences. In some embodiments, the human IGH variable region sequence comprises nucleotide positions 105,862,994 to 106,811,028 of chromosome 14 of the GRCh38.p13 assembly of the human genome. In some embodiments, the human IGH variable region sequence comprises nucleotide positions 105,862,994 to 106,811,028 of chromosome 14 of the GRCh38.p13 assembly of the human genome, minus at least about 50 bp, 100 bp, 500 bp, 1,000 bp, 2,000 bp, 5,000 bp, 7,000 bp, 10,000 bp, 15,000 bp, 20,000 bp or 50,000 bp from the 5′ end the 3′ end, or both. In some embodiments, the human IGH variable region sequence comprises nucleotide positions 105,862,994 to 106,811,028 of chromosome 14 of the GRCh38.p13 assembly of the human genome, and at least about 50 bp, 100 bp, 500 bp, 1,000 bp, 2,000 bp, 5,000 bp, 7,000 bp, 10,000 bp, 15,000 bp, 20,000 bp or 50,000 bp of additional flanking sequence at the 5′ end the 3′ end, or both. In some embodiments, the human IGH variable region sequence comprises nucleotide positions 105,862,994 to 106,811,028 of chromosome 14 of the GRCh38.p13 assembly of the human genome, and one or modifications thereto. Exemplary modifications include, but are not limited to, deletions such as the deletion of one or more V, D or J segments, insertions, such as the insertion of a marker, rearrangements, or a combination thereof.

In some embodiments, the template sequence comprises a sequence of a T cell receptor subunit (TCR). The T-cell receptor (TCR) is a protein complex found on the surface of T cells, or T lymphocytes,[1] that is responsible for recognizing fragments of antigen as peptides bound to major histocompatibility complex (MI-C) molecules. The TCR comprises a disulfide-linked membrane bound heterodimeric protein, which in most cases is composed of highly variable a and β chains expressed as part of a complex with the invariant CD3 chain molecules (CD3δ, CD3ε, CD3γ and CD3ζ. T cells expressing these two chains are referred to as α:β (or αβ) T cell. A small number of T cells express an alternate receptor, formed by variable y and a chains, referred as γσ T cells. TCR development occurs through a lymphocyte specific process of gene recombination, which assembles a final sequence from a large number of potential segments, which occurs through recombination of TCR gene segments in T cells in the thymus. The TCRα gene locus contains variable (V) and joining (J) gene segments (Vβ and Jβ), whereas the TCR locus contains a D gene segment in addition to Vα and Jα segments. Accordingly, the α chain is generated from VJ recombination and the β chain is involved in VDJ recombination. This is similar for the development of γδ TCRs, in which the TCRγ chain is involved in VJ recombination and the TCRδ gene is generated from VDJ recombination. The TCR α chain gene locus consists of 46 variable segments, 8 joining segments and the constant region. The TCR β chain gene locus consists of 48 variable segments followed by two diversity segments, 12 joining segments and two constant regions. A template sequence comprising a sequence of any of the TCR subunits described herein, a subsequence thereof, or a combination thereof, is envisaged as within the scope of the instant disclosure. In some embodiments, the template sequence comprises a TCR alpha chain variable region sequence (encoded by the T-cell receptor alpha locus, or TRA), a TCR beta chain variable region sequence (encoded by the T-cell receptor beta locus, or TRB), a TCR gamma variable region sequence (encoded by the T-cell receptor gamma locus, or TRG), or a TCR delta variable region sequence (encoded by the T-cell receptor delta locus, or TRD).

In some embodiments, the template sequence comprises a sequence encoding an antibody, or an antigen binding fragment.

As used herein, the term “antibody” refers to an immunoglobulin molecule that specifically binds to, or is immunologically reactive with, a particular antigen, and includes polyclonal, monoclonal, genetically engineered, and otherwise modified forms of antibodies, including but not limited to chimeric antibodies, humanized antibodies, heteroconjugate antibodies (e.g., bi- tri- and quad-specific antibodies, diabodies, triabodies, and tetrabodies), and antigen binding fragments of antibodies, including, for example, Fab′, F(ab′)₂, Fab, Fv, rlgG, and scFv fragments. Unless otherwise indicated, the term “monoclonal antibody” (mAb) is meant to include both intact molecules, as well as antibody fragments (including, for example, Fab and F(ab′)₂ fragments) that are capable of specifically binding to a target protein. As used herein, the Fab and F(ab′)₂ fragments refer to antibody fragments that lack the Fc fragment of an intact antibody. Examples of these antibody fragments are described herein.

The term “antigen-binding fragment,” as used herein, refers to one or more fragments of an antibody that retain the ability to specifically bind to a target antigen. The antigen-binding function of an antibody can be performed by fragments of a full-length antibody. The antibody fragments can be, for example, a Fab, F(ab′)2, seFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the V_{L}, VII, CL, and CH1 domains; (ii) a F(ab′)2 fragment, a bivalent fragment containing two Fab fragments linked by a disulfide bridge at the hinge region; (iii) a Fd fragment consisting of the VH and CL domains; (iv) a Fv fragment consisting of the V_{L} and VI domains of a single arm of an antibody, (v) a dAb including VH and V_{L} domains; (vi) a dAb fragment that consists of a VH domain (see, e.g., Ward et al., Nature 341:544-546, 1989); (vii) a dAb which consists of a VH or a V_{L} domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, V_{L} and VH, are coded for by separate genes, they can be joined, using recombinant methods, by a linker that enables them to be made as a single protein chain in which the V_{L}, and VH regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art.

As used herein, the term “complementarity determining region” (CDR) refers to a hypervariable region found both in the light chain and the heavy chain variable domains of an antibody. The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each contain four framework regions that primarily adopt a sheet configuration, connected by three CDRs, which form loops that connect, and in some cases form part of, the P-sheet structure. The CDRs in each chain are held together in close proximity by the framework regions in the order FR1-CDRI-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, Md., 1987). As used herein, numbering of immunoglobulin amino acid residues is performed according to the immunoglobulin amino acid residue numbering system of Kabat et al., unless otherwise indicated.

In some embodiments, the antibody, or antigen binding fragment, comprises a human antibody or antigen binding fragment. In some embodiments, the antibody or antigen binding fragment is humanized.

The person of ordinary skill in the art will understand that the template sequence can also include sequences necessary for the expression of a gene, such as an antibody, in a particular tissue, cell type or organism. Such sequences include, but are not limited to, promoters, enhancers, untranslated sequences such as the 5′ and 3′ untranslated regions of a messenger RNA (mRNA), polyadenylation (polyA) sequences, introns, internal ribosome entry sites (IRES) and the like. The selection of appropriate sequences will be apparent to the person of ordinary skill in the art.

In some embodiments, the template sequence comprises a promoter. In some embodiments, the promoter comprises an endogenous promoter, i.e. the promoter is the promoter normally associated with a gene contained within the template sequence. In some embodiments, the promoter is not an endogenous promoter, for example a promoter isolated or derived from another gene or organism than the gene in the template sequence to which the promoter is operably linked. For example, the template sequence comprises a sequence encoding an antibody or antigen binding fragment operably linked to a promoter that is not an immunoglobulin promoter. In some embodiments, the promoter is a constitutive promoter, an inducible promoter, or a tissue specific promoter. In some embodiments, the promoter is isolated or derived from a mammalian gene, for example a gene expressed in a lymphocyte.

Exemplary promoters which can be used to express a gene of the template sequence include, but are not limited to, the SV40 early promoter region, the promoter contained in the 3′ long terminal repeat of Rous sarcoma virus, the regulatory sequences of the metallothionein gene, the tetracycline (Tet) promoter, the promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: the elastase I gene control region which is active in pancreatic acinar cells; the insulin gene control region which is active in pancreatic beta cells, the immunoglobulin gene control region which is active in lymphoid cells, the mouse mammary tumor virus control region which is active in testicular, breast, lymphoid and mast cells, the albumin gene control region which is active in liver, the alpha-fetoprotein gene control region which is active in liver, the alpha I-antitrypsin gene control region which is active in the liver, the beta-globin gene control region which is active in myeloid cells, the myelin basic protein gene control region which is active in oligodendrocyte cells in the brain, the myosin light chain-2 gene control region which is active in skeletal muscle, the neuronal-specific enolase (NSE) which is active in neuronal cells, the brain-derived neurotrophic factor (BDNF) gene control region which is active in neuronal cells, the glial fibrillary acidic protein (GFAP) promoter which is active in astrocytes the and gonadotropic releasing hormone gene control region which is active in the hypothalamus.

### Target Chromosome

The disclosure provides target chromosomes, comprising target sequences, for use in the methods described herein

As used herein, a “target chromosome” refers to a chromosome containing a “target sequence,” or, in those cases where there is no significant deletion of target sequence by insertion of the template sequence, a “target location.” The target sequence refers to the sequence of the target chromosome which is deleted by insertion of the template sequence using the methods described herein. The target location refers to the location in the target chromosome at which the template sequence is inserted (for insertions) or joined thereto (for chromosomal translocations or rearrangements).

The target chromosome can be isolated or derived from any suitable source. In some embodiments, the target chromosome is from a eukaryote. In some embodiments, the eukaryote is a vertebrate, such as a bird, reptile or mammal. In some embodiments, the target chromosome is from a mouse, rat, rabbit, guinea pig, hamster, sheep, goat, donkey, cow, horse, camel, monkey or chicken. In some embodiments, the target chromosome is from a mouse. In some embodiments, the target chromosome is from a rat. In some embodiments, the target chromosome is from a monkey.

In some embodiments, the template chromosome and the target chromosome are from different species. For example, the template chromosome is from a human, and the target chromosome is from a mouse. In some embodiments, template chromosome and the target chromosome are from the same species.

In some embodiments, the target chromosome is an artificial chromosome.

In some embodiments, the target chromosome is a naturally occurring chromosome.

In some embodiments, the target chromosome comprises one or modifications to a naturally occurring chromosome. Modifications include, inter alia, insertions of sequences, deletions, and rearrangements. Examples of sequences inserted in a target chromosome include, inter alia, markers, promoters, cDNA sequences, non-coding and the like. Suitable makers include selectable markers such as those disclosed in Table 3, as well as detectable markers such as GFP, mCherry and the like.

In some embodiments, the target chromosome comprises an endonuclease site located 5′ of the template sequence. In some embodiments, the target chromosome comprises an endonuclease site located 3′ of the target sequence. In some embodiments, the endonuclease site is located immediately adjacent to the target sequence. In some embodiments, the endonuclease site is located near the target sequence.

In some embodiments, the target chromosome comprises an endonuclease site on either side of the target sequence. For example, the target chromosome comprises a first endonuclease site located 5′ of the target sequence and a second endonuclease site located 3′ of the target sequence. In some embodiments, both the first and second endonuclease sites are recognized and cleaved by the same endonuclease. For example, both the first and second endonuclease sites comprise the same DNA sequence, that is recognized by the same endonuclease. In some embodiments, the first endonuclease site is cleaved by a first endonuclease, and the second endonuclease site is cleaved by a second endonuclease. For example, the first and second endonuclease sites comprise different DNA sequences that recognized by two different zinc finger nucleases (ZFNs), or two different CRISPR/Cas target sequences that are recognized by CRISPR/Cas ribonucleoprotein complexes comprising guide nucleic acids (gNAs) comprising different targeting sequences. In some embodiments, the first and/or second endonuclease site is located immediately adjacent to the target sequence. In some embodiments, the first and/or second endonuclease site is located near the target sequence.

An endonuclease site that is within 5 basepairs (bp), within 10 bp, within 15 bp, within 20 bp, within 30 bp, within 40 bp, within 50 bp, within 70 bp, within 80 bp, within 90 bp, within 100 bp, within 120 bp, within 140 bp, within 160 bp, within 180 bp, within 200 bp, within 250 bp, within 300 bp, within 400 bp or within 500 bp of the template sequence can be considered to be near the target sequence.

In some embodiments, the target chromosome comprises one or more sequences of homology arms of nucleic acid molecules used to facilitate homology directed repair. In some embodiments, the target chromosome comprises a sequence of a homology arm located 5′ of the target sequence. In some embodiments, the target chromosome comprises, from 5′ to 3′, a homology arm sequence, an endonuclease site, and the target sequence. In some embodiments, the target chromosome comprises a sequence of a homology arm located 3′ of the target sequence. In some embodiments, the target chlorosome comprises, from 5′ to 3′, the target sequence, an endonuclease site, and the homology arm sequence. In some embodiments, the endonuclease site is located between the homology arm sequence and the target sequence.

In some embodiments, the target chromosome comprises a first homology arm sequence 5′ of the target sequence, and a second homology arm sequence 3′ of the target sequence. I.e., the target chromosome comprises homology arms both upstream and downstream of the target sequence. In some embodiments, the first homology arm is a 5′ homology arm of a first nucleic acid molecule comprising from 5′ to 3′, the first homology arm, a sequence of at least a first marker, and 3′ homology arm comprising a nucleotide sequence upstream of the 5′ end of the template sequence. In some embodiments, the second homology arm is a 3′ homology arm of a second nucleic acid molecule comprising from 5′ to 3′, 5′ homology arm comprising a nucleotide sequence downstream of the 3′ end of the template sequence, a sequence of at least a second marker, and the second homology arm. In some embodiments, the target chromosome comprises, from 5′ to 3′, the first homology arm sequence, the first endonuclease site, the target sequence, the second endonuclease site, and the second homology arm sequence.

In some embodiments, the first and/or second homology arm sequence of the target chromosome is located immediately adjacent to the first and/or second endonuclease site. In some embodiments, the first homology arm sequence is located immediately adjacent to the first endonuclease site, and the second homology arm sequence is located immediately adjacent to the second endonuclease site, wherein the first endonuclease site is between the first homology arm and the target sequence, and the second endonuclease site is between the target sequence and the second homology arm.

In some embodiments, the first and/or second homology arm sequence is located near the target sequence. An endonuclease site that is within 5 bp, within 10 bp, within 15 bp, within 20 bp, within 30 bp, within 40 bp, within 50 bp, within 70 bp, within 80 bp, within 90 bp, within 100 bp, within 120 bp, within 140 bp, within 160 bp, within 180 bp, within 200 bp or within 250 bp of the target sequence can be considered to be near the target sequence.

In some embodiments, the target chromosome comprises, from 5′ to 3′, the first homology arm, the first endonuclease site, the target sequence, the second endonuclease site, and the second homology arm.

In some embodiments, little or no sequence of the target chromosome is deleted when the template sequence is inserted, and the target sequence is referred to interchangeably herein as a “target site” or “target location.” The person of ordinary skill will appreciate that, in these cases, the arrangement of homology arms and endonuclease sites is similar to those described supra, except that the homology arms flank an endonuclease site at a target location, rather than a target sequence itself flanked by endonuclease sites. In some embodiments, the target chromosome comprises, from 5′ to 3′, a sequence of a first homology arm, an endonuclease site, and a sequence of a second homology arm. In some embodiments, the first homology arm is a 5′ homology arm of a first nucleic acid molecule comprising from 5′ to 3′, the first homology arm, a sequence of at least a first marker, and 3′ homology arm comprising a nucleotide sequence upstream of the 5′ end of the template sequence. In some embodiments, the second homology arm is a 3′ homology arm of a second nucleic acid molecule comprising from 5′ to 3′, 5′ homology arm comprising a nucleotide sequence downstream of the 3′ end of the template sequence, a sequence of at least a second marker, and the second homology arm.

In some embodiments, the template sequence is joined to the target sequence to generate a chromosomal rearrangement or translocation. In some embodiments, the target chromosome comprises, from 5′ to 3′, a target chromosome homology arm sequence, and endonuclease site. In some embodiments, the target chromosome homology arm comprises a 5′ homology arm of a nucleic acid molecule comprising, from 5′ to 3′, the target sequence homology arm, at least one marker, and 3′ homology arm comprising a nucleotide sequence upstream of the 5′ end of the template sequence. In some embodiments, the target chromosome comprises, from 5′ to 3′, an endonuclease site and a target chromosome homology arm sequence. In some embodiments, the target chromosome homology arm comprises the 3′ homology arm of a nucleic acid molecule comprising, from 5 to 3′, a 5′ homology arm comprising a nucleotide sequence downstream of the 3′ end of the template sequence, at least a first marker, and the target sequence homology arm.

In some embodiments, the first and/or second homology arm sequences of the target chromosome are between about 20 and 2,000 bp, between about 50 and 1,500 bp, between about 100 and 1,400 bp, between about 150 and 1,300 bp, between about 200 and 1,200 bp, between about 300 and 1,100 bp, between about 400 and 1,000 bp, or between about 500 and 900 bp, or between about 600 bp and 800 bp in length. In some embodiments, the homology sequences of the target chromosome are between about 400 and 1,500 bp in length. In some embodiments, the homology sequences of the target chromosome are between about 500 and 1,300 bp in length. In some embodiments, the homology sequences of the target chromosome are between about 600 and 1,000 bp in length.

### Target Sequence or Target Location

The target chromosome comprises the target sequence or location into which the template sequence is inserted, or to which the template sequence is joined by the methods described herein. The target sequence can be located at any suitable location on the target chromosome.

The target sequence can be isolated or derived from any suitable source. In some embodiments, the target sequence and the template sequence are from different species. For example, the template sequence is from a human, and the target sequence is from a mouse. In some embodiments, target sequence and the template sequence are from the same species.

In some embodiments, the target sequence comprises a naturally occurring sequence. In some embodiments, the target sequence comprises one or modifications to a naturally occurring sequence. Modifications include, inter alia, insertions of sequences such as artificial sequences or markers, deletions, and rearrangements. In some embodiments, the target sequence comprises an artificial sequence. In some embodiments, the target sequence comprises both naturally occurring and artificial sequences. Exemplary artificial sequences include, inter alia, markers, cDNA sequences, promoters, and recombinant sequences. Exemplary markers include, but are not limited to, the selectable markers disclosed in Table 3 below, as well as detectable markers such as green fluorescent protein (GFP), mCherry and the like.

In some embodiments, the target sequence is from a eukaryote. In some embodiments, the eukaryote is a vertebrate, such as a bird, reptile or mammal. In some embodiments, the template sequence comprises a mouse, rat, rabbit, guinea pig, hamster, sheep, goat, donkey, cow, horse, camel, monkey or chicken sequence. In some embodiments, the target sequence comprises a mouse sequence. In some embodiments, the target sequence comprises a rat sequence. In some embodiments, the target sequence comprises a monkey sequence.

In some embodiments, the target sequence is at least 25 KB, at least 50 KB, at least 100 KB, at least 200 KB, at least 400 KB, at least 500 KB, at least 600 KB, at least 700 KB, at least 800 KB, at least 900 KB, at least 1 MB, at least 2 MB, at least 3 MB, at least 4 MB, at least 5 MB, at least 6 MB, at least 7 MB, at least 8 MB, at least 9 MB, at least 10 MB, at least 15 MB, at least 20 MB, at least 25 MB, at least 30 MB, at least 40 MB, at least 50 MB, at least 60 MB, at least 70 MB, at least 80 MB, at least 90 MB, at least 100 MB, at least 120 MB, at least 140 ^{M}B, at least 160 MB, at least 180 MB, at least 200 MB, at least 220 MB, or at least 250 MB in length. In some embodiments, the target sequence is at least 50 KB, at least 100 KB, at least 200 KB, at least 500 KB, at least 700 KB, at least 1 MB, at least 2 MB, at least 3 MB, at least 4 MB, at least 5 MB, at least 6 MB, at least 7 MB, at least 8 MB, at least 9 MB, at least 10 MB, at least 20 MB, at least 30 MB, at least 40 MB, or at least 50 MB in length. In some embodiments, the target sequence is at least 1 M1B in length. In some embodiments, the target sequence is at least 2 MB in length. In some embodiments, the target sequence is at least 3 MB in length. In some embodiments, the target sequence is at least 4 MB in length. In some embodiments, the target sequence is at least 5 MB in length. In some embodiments, the target sequence is at least 10 MB in length. In some embodiments, the target sequence is at least 20 MB in length.

In some embodiments, the target sequence is between 50 KB and 250 M3, 50 KB and 100 MB, 50 KB and 50 MB, 50 KB and 20 MB, 50 KB and 10 MB, 50 KB and 5 MB, 50 KB and 3 MB, 50 KB and 2 MB, 50 KB and 1 MB, 100 KB and 200 MB, 100 KB and 100 MB, 100 KB and 50 MB, 100 KB and 20 MB, 100 KB and 10 MB, 100 KB and 5 MB, 100 KB and 3 MB, 100 KB and 21 MB, 100 KB and 1 MB, 100 KB and 500 KB, 200 KB and 100 MB, 200 KB and 50 MB, 200 KB and 20 MB, 200 KB and 10 MB, 200 KB and 5 MB, 200 KB and 3 MB, 200 KB and 2 MB, 200 KB and 1 MB, 200 KB and 500 KB, 500 KB and 100 MB, 500 KB and 50 MB, 500 KB and 20 MB, 500 KB and 10 MB, 500 KB and 5 MB, 500 KB and 3 MB, 500 KB and 2 MB, 500 KB and 1 MB, 1 MB and 100 MB, 1 MB and 50 MB, 1 MB and 20 MB, 1 MB and 10 MB, 1 MB and 5 MB, 1 MB and 3 MB, 1 MB and 2 MB, 3 MB and 100 MB, 3 MB and 50 MB, 3 MB and 20 MB, 3 MB and 10 MB, 3 MB and 5 MB, 5 MB and 100 MB, 5 MB and 50 MB. 5 MB and 20 MB, 5 MB and 10 MB, 10 MB and 100 MB, 10 MB and 50 MB, or 10 MB and 20 MB, in length. In some embodiments, the target sequence is between 200 KB and 50 MB, between 1 MB and 20 MB, between 1 MB and 10 MB, between 1 MB and 5 MB, between 1 MB and 3 MB, between 3 MB and 20 MB, between 3 MB and 10 MB, between 3 MB and 7 MB, or between 3 MB and 5 MB in length. In some embodiments, the target sequence is between 1 MB and 10 MB in length. In some embodiments, the target sequence is between 1 13 and 5 MB in length. In some embodiments, the target sequence is between 3 MB and 5 MB in length.

In some embodiments, the target sequence comprises sequences of one or more genes. In some embodiments, the target sequence comprises sequences of multiple genes. In some embodiments, the target sequence comprises the sequence of at least 2, 3, 4, 5, 6, 7, 8, 9, 10, 15, 20, 25, 30, 35, 40, 45, 50, 60, 70, 80, 90, 100, 150, 200, 250, 300, 350, 400, 450, 500, 600, 700, 800, 900, 1000, 1500 or 2000 genes.

In some embodiments, the target sequence comprises a sequence homologous to the template sequence. For example, the template chromosome is a human chromosome comprising a human template sequence comprising one or more of the genes described in Tables 1 and 2, supra, while the target chromosome is a mouse chromosome comprising a mouse target sequence, and the mouse target sequence comprises the mouse sequence homologous to the human template sequence. As a further example, the template chromosome is a human chromosome comprising a human IGH sequence, while the target chromosome is a mouse chromosome, and the target sequence comprises the homologous mouse Igh sequence. As a yet further example the template chromosome is a human chromosome comprising a human TCR sequence, while the target chromosome is a mouse chromosome, and the target sequence comprises the homologous mouse TCR sequence.

In some embodiments, the target chromosome is from a mouse, rat, rabbit, guinea pig, hamster, sheep, goat, donkey, cow, horse, camel, monkey or chicken, and the target sequence comprises the mouse, rat, rabbit, guinea pig, hamster, sheep, goat, donkey, cow, horse, camel, monkey or chicken homolog of the template sequence.

In some embodiments, the target sequence comprises a sequence of a mouse, rat, rabbit, guinea pig, hamster, sheep, goat, donkey, cow, horse, camel, monkey or chicken gene. All mouse, rat, rabbit, guinea pig, hamster, sheep, goat, donkey, cow, horse, camel, monkey or chicken genes are envisaged within the scope of the instant disclosure. Without wishing to be bound by theory, transfer of human genes involved in disease pathogenesis, or that are potential therapeutic targets, to a model organism such as a mouse, rat, rabbit, guinea pig, hamster, sheep, goat, donkey, cow, horse, camel, monkey or chicken can facilitate research into the disease and development of suitable therapies. In some embodiments, the target sequence comprises a mouse sequence homologous to a human template sequence. In some embodiments, the target sequence comprises a rat sequence homologous to a human template sequence. In some embodiments, the target sequence comprises a monkey sequence homologous to a human template sequence.

In some embodiments, the target sequence comprises an immunoglobin sequence, such as a mouse immunoglobulin sequence. In some embodiments, the target sequence comprises a mouse Igh sequence. Mouse Igh spans nucleotide positions 1112,947,269 to 116,248,693 of chromosome 12 the GRCm39 assembly of the mouse genome. The skilled artisan will appreciate that mouse Igh sequences with 5′ and 3′ boundaries that deviate from those described supra, for example by at least 100 bp, 500 bp, 1,000 bp, 2,000 bp, 5,000 bp, 10,000 bp or more are suitable template sequences.

In some embodiments, the target sequence comprises a mouse Igh variable region sequence. In some embodiments, the mouse Igh variable region sequence comprises a sequence encoding mouse homologs of the V_{H}, D_{H} and J_{H}1-6 gene segments and intervening non-coding sequences. In some embodiments, the mouse Igh variable region sequence comprises nucleotide positions 113,391,842 to 115,973,952 of chromosome 12 of the GRCm39 assembly of the mouse genome. In some embodiments, the mouse Igh variable region sequence comprises nucleotide positions 113,391,842 to 115,973,952 of chromosome 12 of the GRCm39 assembly of the mouse genome, minus at least about 50 bp, 100 bp, 500 bp, 1,000 bp, 2,000 bp, 5,000 bp, 7,000 bp, 10,000 bp, 15,000 bp, 20,000 bp or 50,000 bp from the 5′ end the 3′ end, or both. In some embodiments, the human IGH variable region sequence comprises nucleotide positions 113,391,842 to 115,973,952 of chromosome 12 of the GRCm39 assembly of the mouse genome, and at least about 50 bp, 100 bp, 500 bp, 1,000 bp, 2,000 bp, 5,000 bp, 7,000 bp, 10,000 bp, 15,000 bp, 20,000 bp or 50,000 bp of additional flanking sequence at the 5′ end the 3′ end, or both. In some embodiments, the mouse Igh variable region sequence comprises nucleotide positions 113,391,842 to 115,973,952 of chromosome 12 of the GRCm39 assembly of the mouse genome, and one or modifications thereto. Exemplary modifications include, but are not limited to, deletions such as the deletion of one or more V, D or J segments, insertions, such as the insertion of a marker, rearrangements, or a combination thereof. In some embodiments, the target sequence comprises a mouse Igl variable region sequence. In some embodiments, the target sequence comprises a mouse Igk variable region sequence. In some embodiments, the template sequence comprises a human IGL variable region sequence. In some embodiments, the template sequence comprises a human IGK variable region sequence.

In some embodiments, for example those embodiments where little or no target chromosomal sequence is deleted by the methods described herein, the target chromosome comprises a target location. The target location is the location into which the template sequence is inserted, or to which the template sequence is joined. Any location on the target chromosome may be a suitable location. In some embodiments, the target location comprises an endonuclease site for generating a double stranded break at the target location.

### Engineered Chromosomes

The disclosure provides engineered chromosomes produced by the methods described herein.

In some embodiments, the engineered chromosomes comprise a mouse chromosome comprising one or more humanized sequences. In some embodiments, the humanized sequence comprises one or more genes linked to a disease or disorder in humans, such as a gene linked to a genetic disease or disorder, or an oncogene. In some embodiments, the engineered chromosomes comprise a rat chromosome comprising one or more humanized sequences. In some embodiments, the engineered chromosomes comprise a monkey chromosome comprising one or more humanized sequences.

In some embodiments, the engineered chromosome comprises a mouse chromosome in which one or more immunoglobulin sequences have been humanized. In some embodiments, the immunoglobulin sequence comprises an IGH sequence, such as the IGH variable regions. In some embodiments, the engineered chromosome comprises mouse chromosome 12, wherein mouse Igh variable regions have been replaced with the human IGH variable regions from chromosome 14. In some embodiments, the mouse Igh variable region comprises V_{H}, D_{H} and J_{H}1-6 gene segments and intervening non-coding sequences. In some embodiments, the human IGH variable region comprises V_{H}, D_{H} and J_{H}1-6 gene segments and intervening non-coding sequences. In some embodiments, the engineered chromosome comprises mouse chromosome 12, wherein mouse Igh variable regions comprising approximately a nucleotide sequence of 113,391,842 to 115,973,952 of chromosome 12 of the GRCm39 assembly of the mouse genome has been replaced with human IGH variable regions comprising approximately a nucleotide sequence of 105,862,994 to 106,811,028 of chromosome 14 of the GRCh38.p13 assembly of the human genome. In some embodiments, the engineered chromosome is a mouse chromosome 6 comprising a sequence of a human IGK variable region in place of a mouse Igk variable region.

In some embodiments, the mouse Igk variable region sequence comprises a sequence encoding mouse Vₖ, and Jₖ₁₋₅ gene segments and intervening non-coding sequences. In some embodiments, the template sequence comprises a human IGK variable region sequence. In some embodiments, the human ICK variable region sequence comprises a sequence encoding human Vₖ, and Jₖ₁₋₅ gene segments and intervening non-coding sequences.

### Nucleic Acid Molecules, Plasmids and Vectors

The disclosure provides nucleic acid molecules for use in the methods described herein. Nucleic acid molecules, sometimes referred to as polynucleotides, refer to chains of linked nucleotides that make up a single molecule. The nucleic acid molecules of the disclosure can be deoxyribonucleic acids (DNA), or ribonucleic acids (RNA). Exemplary nucleic acid molecules of the disclosure comprise homology arms specific to or adjacent to both the target and template sequences in order to facilitate insertion of the template sequence into the target sequence, or joining of the template and target sequences by double strand break repair.

The disclosure provides nucleic acid molecules comprising the homology arms specific to the target and template chromosomes, which facilitate the HDR-mediated chromosomal rearrangements described herein. In some embodiments, the nucleic acid molecule comprises, from 5′ to 3′, a 5′ homology arm comprising a nucleotide sequence upstream of the 5′ end of the target sequence, at least a first marker, and a 3′ homology arm comprising a nucleotide sequence upstream of the 5′ end of the template sequence. In some embodiments, the nucleic acid molecule comprises, from 5′ to 3′, a 5′ homology arm comprising a nucleotide sequence downstream of the 3′ end of the template sequence, at least a second marker, and a 3′ homology arm comprising a nucleotide sequence downstream of the 3′ end of the target sequence.

The disclosure provides vectors comprising the nucleic acid molecules described herein. A vector, according to the present disclosure, is a nucleic acid molecule capable of transporting other nucleic acids to which it has been linked. A plasmid is, e.g., a type of vector. Vector sequences include, inter alia, sequences necessary for the production of the vector from a host cell such as a bacterium, such as an origin or replication, and selectable markers.

In some embodiments, the vector is a plasmid. In some embodiments, the plasmid comprises, from 5′ to 3′, a 5′ homology arm comprising a nucleotide sequence upstream of the 5′ end of the target sequence, at least a first marker, and a 3′ homology arm comprising a nucleotide sequence upstream of the 5′ end of the template sequence. In some embodiments, the plasmid comprises, from 5′ to 3′, a 5′ homology arm comprising a nucleotide sequence downstream of the 3′ end of the template sequence., at least a second marker, and a 3′ homology arm comprising a nucleotide sequence downstream of the 3′ end of the target sequence.

In some embodiments, the vector comprises a sequence of a homology arm located at or near the 5′ end of the template sequence. In some embodiments, the homology arm is located upstream, i.e. 5′ of, the template sequence. In some embodiments, the vector comprises a sequence of a homology arm located at or near the 3′ end of the template sequence. In some embodiments, the homology arm is located downstream, i.e. 3′ of, the template sequence. In some embodiments, the sequence of the template homology arm in the vector is identical to, or substantially identical to, the sequence of the homology arm in the template sequence.

In some embodiments, the vector comprises a sequence of a homology arm located 5′ of the target sequence or location, i.e. upstream of the target sequence or location. In some embodiments, the vector comprises a sequence of a homology arm located 3′ of the target sequence or location, i.e. downstream of the target sequence or location.

The skilled artisan will understand that there can be some degree of mismatch between the homology arm sequence in the vector, and the equivalent sequence in the template or target chromosome, and the vector will still facilitate repair of the double strand break in the template or target chromosome from the vector. For example, a vector homology arm sequence that is at least 95% identical, at least 96% identical, at least 97% identical, at least 98% identical or at least 99% identical or is identical to the equivalent sequence in the template chromosome will be suitable for the methods of the disclosure.

In some embodiments, the nucleic acid molecules, plasmids, or vectors described herein comprise one or more endonuclease sites.

In some embodiments, the disclosure provides (i) a first nucleic acid molecule comprising from 5′ to 3′, a 5′ homology arm comprising a nucleotide sequence upstream of the 5′ end of the target sequence, at least a first marker, and a 3′ homology arm comprising a nucleotide sequence upstream of the 5′ end of the template sequence; and (ii) a second nucleic acid molecule comprising from 5′ to 3′, a 5′ homology arm comprising a nucleotide sequence downstream of the 3′ end of the template sequence, at least a second marker, and a 3′ homology arm comprising a nucleotide sequence downstream of the 3′ end of the target sequence. In some embodiments, the first and second nucleic acid molecules are plasmids. In some embodiments, the first nucleic acid molecule comprises, from 5′ to 3′, the 5′ homology arm comprising a nucleotide sequence upstream of the 5′ end of the target sequence, an first endonuclease site, at least a first marker, a second endonuclease site, and the 3′ homology arn comprising a nucleotide sequence upstream of the 5′ end of the template sequence, wherein the first and second endonuclease sites overlap the homology arms such that the first and second endonuclease sites on the nucleic acid molecule, and the corresponding endonuclease sites on the template and target chromosomes are cut by the same endonucleases. In some embodiments, the second nucleic acid molecule comprises from 5′ to 3′, the 5′ homology arm comprising a nucleotide sequence downstream of the 3′ end of the template sequence, a third endonuclease site, at least a second marker, a fourth endonuclease site, and the 3′ homology arm comprising a nucleotide sequence downstream of the 3′ end of the target sequence, wherein the second and third endonuclease sites overlap the homology arms such that the third and fourth endonuclease sites on the nucleic acid molecule, and the corresponding endonuclease sites on the template and target chromosomes are cut by the same endonucleases. In some embodiments, the first and second markers are not the same marker. In some embodiments, the first marker on the first nucleic acid molecule comprises a combination of a selectable marker and a detectable marker.

In some embodiments, the first marker comprises eGFP and Puromycin resistance. In some embodiments, the second marker comprises a selectable marker. In some embodiments, the second marker comprises Hygromycin resistance.

In some embodiments, the homology arm sequence on the nucleic acid molecule corresponds to a sequence that is located near the template sequence, the target sequence or the target location. An homology arm that is within 0 bp, 5 basepairs (bp), within 10 bp, within 15 bp, within 20 bp, within 30 bp, within 40 bp, within 50 bp, within 70 bp, within 80 bp, within 90 bp, within 100 bp, within 120 bp, within 140 bp, within 160 bp, within 180 bp, within 200 bp or within 250 bp of the template sequence, target sequence, or target location can be considered to be near said sequence.

In some embodiments, the nucleic acid molecule homology sequences corresponding to template or target chromosome sequence are between about 20 and 2,000 bp, between about 50 and 1,500 bp, between about 100 and 1,400 bp, between about 150 and 1,300 bp, between about 200 and 1,200 bp, between about 300 and 1,100 bp, between about 400 and 1,000 bp, or between about 500 and 900 bp, or between about 600 bp and 800 bp in length. In some embodiments, the nucleic acid molecule homology sequences are between about 400 and 1,500 bp in length. In some embodiments, the nucleic acid molecule homology sequences are between about 500 and 1,300 bp in length. In some embodiments, the nucleic acid molecule homology sequences are between about 600 and 1,000 bp in length.

In some embodiments, the nucleic acid molecule comprises a marker suitable for expression in a mammalian cell. In some embodiments, the marker is between the homology arms in the nucleic acid molecule, whereby the marker is inserted into the target sequence. In some embodiments, the marker is a selected able marker. Suitable selected markers include Dihydrofblate reductase (DHFR), Glutamine synthase (GS), Puromycin aceetylransferase, Blasticidin deaminase, Histidinol dehydragenase, Hygromycin phasphoiransferase (hph), Bleamycin resistance gene, Aminoglycosidase phosphorransferase (neomycin resistance gene), and are described in further detail in Table 3 below.

In some embodiments, the marker comprises an detectable marker (or reporter). Detectable markers include, but are not limited to, enzymes that mediate luminescence reactions (luxA, luxB, luxAB, luc, rue, nluc), enzymes that mediate colorimetric reactions (lacZ, HRP), and fluorescent proteins such as green fluorescent protein (GFP), eGFP, yellow fluorescent protein (YFP), red fluorescent protein (RFP), cyan fluorescent protein (CFP), blue fluorescent protein (BFP), dsRed, mCherry, tdTomato, near-infrared fluorescent proteins, and the like. Selection of a suitable detectable marker will be known to persons of ordinary skill in the art.

Markers can be expressed using any suitable promoter known in the art, including, but not limited to, the cytomegalovirus early (CMV) promoter, the PGK promoter, and the EF1a promoter.

TABLE 3
Selectable Markers

| Selectable Marker | Selective Reagent |
|---|---|
| Dihydrofolate reductase (DHFR) | Methionine |
| | sulphoximine (MSX) |
| Glutamine synthase (GS) | Methotrexate (MTX) |
| Puromycin acetyltransferase | Puromycin |
| Blasticidin deaminase | Blasticidin |
| Histidinol dehydrogenase | Histidinol |
| Hygromycin | Hygromycin |
| phosphotransferase (hph) | |
| Bleomycin resistance gene | Bleomycin |
| Aminoglycosidase phosphotransferase | Neomycin (G418) |

In some embodiments, for example those embodiments of the methods where two nucleic acid molecules are used, a first nucleic acid molecule with a first marker and a second nucleic acid molecule with a second marker, the first or second marker comprises a fluorescent protein operably linked to a promoter capable of expressing the fluorescent protein in the cell. In some embodiments, the fluorescent protein comprises green fluorescent protein (GFP). In some embodiments, the first marker further comprises a selectable marker. In some embodiments, the second marker further comprises a selectable marker. In some embodiments, the selectable marker is selected from the group consisting of Dihydro/late reductase (DHFR), Giutamine synthase (GS), Puromycin acetyltransferase, Blasticidin deaminase, Histidinol dehydrogenase, Hygromycin phosphotransferase (hph), Bleomycin resistance gene and Aminoglycoside phosphotransferase. In some embodiments, the first and second markers are not the same selectable marker. In some embodiments, the first marker comprises GFP operably linked to a promoter capable of expressing the GFP in the cell and Puromycin acetyltransferase, and the second marker comprises Hygromycin phosphotransfQrase.

### Methods of Generating Double Strand Breaks

Provided herein are methods of generating double strand breaks in a template and a target chromosome. The methods provided herein use repair pathways for double strand break repair in a cellular environment to facilitate the transfer of large sequences between chromosomes.

Any methods of generating double strand breaks in DNA sequence known in the art, and any repair pathways that repair those double strand breaks, are envisaged as within the scope of the instant disclosure.

In some embodiments, double strand breaks in the template and target chromosomes are generated using one or more endonucleases. In some embodiments, the endonucleases also cut the one or more nucleic acid molecules comprising homology arms used in the methods described herein. In some embodiments, the one or more endonucleases are selected from the group consisting of a CRISPR/Cas endonuclease and one or more guide nucleic acids (gNAs), one or more zinc finger nucleases (ZFNs), or one or more Transcription Activator-Like Effector Nucleases (TALENs). In some embodiments, double strand breaks in the template and target chromosomes are generated using one or more CRE recombinase to generate chromosomal rearrangement.

Different molecules are able to introduce double and/or single strand breaks into genomic nucleic acids. The nucleases of the present disclosure include, but not limited to, homing endonucleases, restriction enzymes, zinc-finger nucleases or zinc-finger nickases, meganucleases or meganickases, transcription activator-like effector (TALE) nucleases guided, in particular nucleic acid guided nucleases or nickases, such as a RNA-guided nucleases, DNA-guided nucleases, a mega TAL nuclease, a BurrH-nuclease, a modified or chimeric version or variant thereof, and combinations thereof. The RNA-guided nuclease or the RNA-guided nickase are optionally part of a CRISPR-based system.

Nucleases are capable of cleaving phosphodiester bonds between monomers of nucleic acids. Many nucleases participate in DNA repair by recognizing damage sites and cleaving them from the surrounding DNA. These enzymes may be part of complexes. Endonucleases are nucleases that act on central regions of the target molecules. Deoxyribonuclease act on DNAs. Many nucleases involved in DNA repair are not sequence-specific. In the present context, however, sequence-specific nucleases are preferred. In some embodiments, sequence-specific nuclease(s) is/are specific for fairly large strings of nucleotides in the target genome, such as 10 or more nucleotides, or 15, 20, 25, 30, 35, 40, 45 or even 50 or more nucleotides, the ranges of 5-50, 10-50, 15-50, 15-40, 15-30 as target sequences in the target genome are preferred. The larger such a “recognition sequence” the fewer target sites are in a genome and the more specific the cut the nucleases make into the genome is, ergo the cuts become site specific. A site-specific nuclease has generally less than 10, 5, 4, 3, 2 or just a single (1) target site in a genome. Nucleases that have been engineered for altering genomic nucleic acid(s), including by cutting specific genomic target sequences, are referred to herein as engineered nucleases. CRISPR-based systems are one type of engineered nuclease(s). However, such an engineered nuclease can be based on any nuclease described herein.

Endonucleases recognizing sequences larger than 12 base pairs are called meganucleases. Meganucleases/-nickases are endodeoxyribonucleases characterized by a large recognition site (double-stranded DNA sequences of, e.g., 12 to 40 base pairs, such as 20-40 or 30-40 base pairs); as a result this site might only occur once in any given genome.

“Homing endonuclease” are a form of meganucleases and are double stranded DNases that have large, asymmetric recognition sites and coding sequences that are usually embedded in either introns or inteins. Homing endonuclease recognition sites are extremely rare Within the genome so that they cut at very few locations, sometimes a singular location within in the genome (WO2004067736, see also U.S. Pat. No. 8,697,395 B2).

Zinc-finger nucleases/-nickases (ZFNs) are artificial restriction enzymes generated by fusing zinc finger DNA-binding domains to a DNA-cleavage domain. Zinc finger domains can be engineered to target specific desired DNA sequences.

RNA-guided nucleases/-nickases, in particular endonucleases include, for example Cas9 or Cpf1. The CRISPR system has been described in detail. Any CRISPR based system is part of the instant disclosure. In case another RNA-guided endonuclease(s) is/are used, an appropriate guide-RNA, sgRNA or crRNA or other suitable RNA sequences that interacts with the RNA-guided endonuclease and targets to a genomic target site in the genomic nucleic acid can be used.

As used herein, the term “CRISPR associated protein” or “CRJSPPJCas” protein refers to an nucleic acid-guided DNA endonuclease associated with the CRISPR (Clustered Regularly Interspaced Short Palindromic Repeats) type II adaptive immunity system found in certain bacteria, such as Streptococcus pyogenes and other bacteria. CRISPR/Cas proteins, such as Cas9, are not limited to the wild-type (wt) proteins found in bacteria. CRISPR/Cas proteins encompassing mutations to or derivatives of wild type CRISPR/Cas sequences are envisaged as within the scope of the instant disclosure. The original type II CRISPR system from Streptococcus pyogenes comprises the Cas9 protein and a guide RNA composed of two RNAs: a mature CRISPR RNA (crRNA) and a partially complementary trans-acting RNA (tracrRNA). Cas9 unwinds foreign DNA and checks for sites complementary to a 20 base pair spacer region of the guide RNA. Cas9 targeting has been simplified and most Cas-based systems have been engineered to require only one or two chimeric guide RNA(s) or single guide RNA(s) (chiRNA, often also just referred to as guide RNA or gRNA or sgRNA), resulting from the fusion of the crRNA and the tracrRNA. The spacer region may be engineered as required.

As used herein, the term “Cas9 coding sequence” refers to a polynucleotide capable of being transcribed and/or translated, according to a genetic code functional in a host cell/host mammal, to produce a Cas9 protein. The Cas9 coding sequence may be a DNA (such as a plasmid) or an RNA (such as an mRNA).

As used herein, the term CRISPR/Cas ribonucleoprotein refers to a protein/nucleic acid complex consisting of CRISPR/Cas protein and an associated guide nucleic acid. For example, the Cas9 ribonucleoprotein refers to Cas9 in a complex with its associated guide RNA.

In some embodiments, the nuclease is a RNA-guided nuclease. Non-limiting examples of RNA-guided nucleases, including nucleic acid-guided nucleases, for use in the present disclosure include, but are not limited to, CasT, CasIB, Cas2, Cas3, Cas4, Cas5, Cas6, Cas7, Cas8, Cas9, Cas10, CasX, CasY, Cas12a (Cpf1), Cas12b, Cas13a, Csy1, Csy2, Csy3, Cse1, Cse2, CscI, Csc2, Csa5, Csn2, Csm2, Csmn3, Csm4, Csm5, Csm6, Cmr1, Cmr3, Cmr4, Cmr5, Cmr6, CsbI, Csb2, Csb3, Csx17, Csx14, Csx10, Csx16, CsaX, Csx3, Csx1, Csx15, Csf1, Csf2, Csf3, Csf4, Cms1, C2c1, C2c2, C2c3, or a homolog, ortholog, or modified version thereof.

A “megaTAL nuclease/-nickase” refers to an engineered nuclease comprising an engineered TALE DNA-binding domain and an engineered meganuclease or an engineered homing endonuclease. TALE DNA-binding domains can be designed for binding DNA at almost any locus of a nucleic acid sequence in a genome, and cleave the target sequence if such a DNA-binding domain is fused to an engineered meganuclease. Illustrative examples of megaTAL nuclease and design of TALE DNA-binding domains are disclosed in described, for instance by Boissel et al. (MegaTALs: a rare-cleaving nuclease architecture for therapeutic genome engineering (2013), Nucleic Acids Research 42 (4):2591-2601), and references cited therein, all of which are incorporated herein by reference in their entireties. A megaTAL nuclease optionally comprises one or more linkers and/or additional functional domains, e.g. a C-terminal domain (CTD) polypeptide, a N-terminal domain (NTD) polypeptide, an end-processing enzymatic domain of an end-processing enzyme that exhibits 5-3′ exonuclease or 3-5′ exonuclease, or other non-nuclease domains, e.g. a helicase domain.

Transcription activator-like effector (TALE) nucleases/-nickases are restriction enzymes that can be engineered to cut specific sequences of DNA. Transcription activator-like effectors (TALEs) can be engineered to bind to practically any desired DNA sequence, so when combined with a DNA-cleavage domain, DNA can be cut at specific locations.

A “TALE DNA binding domain” is the DNA binding portion of transcription activator-like effectors (TALE or TAL-effectors), which mimics plant transcriptional activators to manipulate the plant transcriptome. TALE DNA binding domains contemplated in some embodiments are engineered de novo or from naturally occurring TALEs, and include, but are not limited to, AvrBs3 from Kanthomonas campestris pv, vesicatoria, Xanthononas gardneri, Xanthoimonas translucens, Kanthoinonas axonopodis, Xanthomonas perforans, Xanthomonas alfalfa, Xanthomonas citri, Xanthomonas euvesicatoria, and Xanthomonas oryzae and brg11 and hpxl7 from Raistonia solanacearurn. Illustrative examples of TALE proteins for deriving and designing DNA binding domains are disclosed in U.S. Pat. No. 9,017,967, and references cited therein, all of which are incorporated herein by reference in their entireties.

A “Burnri-nuclease” refers to a fusion protein having nuclease activity, that comprises modular base-per-base specific nucleic acid binding domains (MBBBD). These domains are derived from proteins from the bacterial intracellular symbiont Burkholderia Rhizoxinica or from other similar proteins identified from marine organisms. By combining together different modules of these binding domains, modular base-per-base binding domains can be engineered for having binding properties to specific nucleic acid sequences, such as DNA-binding domains. Such engineered MBBBD can thereby be fused to a nuclease catalytic domain to cleave DNA at almost any locus of a nucleic acid sequence in a genome. Illustrative examples of BurrH-nucleases and design of MBBBDs are disclosed in WO 2014/018601 and US)2015225465 A1, and references cited therein, all of which are incorporated herein by reference in their entireties.

A related aspect of the present disclosure provides a nucleic acid molecule, such as a vector, suitable for generating a CRISPR/Cas-mediated double-stranded break (DSB) in a cell. In some embodiments, the vector comprises a sequence encoding the CRISPR/Cas protein, e.g. Cas9, and the guide nucleic acid (the Cas9 single guide RNA, or sgRNA), operably linked to suitable promoters for their expression in the cell, as well as other vector components such as an origin or replication and a selectable marker. In some embodiments, the cell is an embryonic stem cell or embryonic hybrid stem cell as described herein.

In accordance with the present disclosure, homologous recombination is facilitated by double strand breaks (DSBs) created by endonucleases. In some embodiments, the endonuclease comprises CRISPR/Cas9 and one or more single guide RNA(s) (“sgRNA” or “gRNA” for short). The person or ordinary skill in the art will be able to select guide RNAs with targeting sequences flanking the template sequence and target sequence, or at the target location, as described for endonuclease sites supra.

In some embodiments, the enzyme can be introduced by introducing nucleic acid molecules, such as vector(s) or coding sequence encoding the CRISPR/Cas protein, and one or more sgRNA(s). In some embodiments, the vector or coding sequence encoding the CRISPR/Cas protein is a CRISPR/Cas mRNA. In some embodiments, the vector or coding sequence encoding the CRISPR/Cas protein is a vector such as plasmid, comprising a DNA sequence encoding the CRISPR/Cas protein and the gRNA. In some embodiments, CRISPR/Cas protein is Cas9. In certain embodiments, isolated CRISPR/Cas protein can be introduced into the cell (e.g., a zygote or an ES cell, through microinjection or electroporation) directly. The CRISPR/Cas protein may be in the form of a CRISPR/Cas ribonucleoprotein, which is a CRISPR/Cas protein/gNA (guide nucleic acid) complex. Or the CRISPR/Cas protein may be without any gNA, such that the CRISPR/Cas protein and the one or more gNAs are co-introduced into the zygote or ES cell to allow the formation of the CRISPR/Cas protein/gNA complex in situ inside the cell. In some embodiments, the CRISPR/Cas protein and the gNA are encoded by a vector, which is introduced into the cell by transfection, electroporation or transduction. In some embodiments, CRISPR/Cas protein is Cas9.

In order to function as an endonuclease for use in the methods of the disclosure, CRISPR/Cas proteins are required to form a functional complex with a gRNA.

According to some embodiments, multiple gNAs are used, each targeting a specific CRISPR/Cas cleavage site. For example, the four gNAs may be used, two with targeting sequences specific to gNA target sequences on either side of the template sequence, and two with targeting sequences specific to gNA target sequences on either side of the target sequence. Alternatively, three gNAs may be used, one with a targeting sequence specific to a gNA target sequence at the target location and two with targeting sequences specific to gNA target sequences on either side of the template sequence. As a yet further example, two gNAs may be used, one with a targeting sequence specific to a gNA target sequence adjacent to the template sequence, and one with a targeting sequence specific to a gNA target sequence adjacent to the target sequence.

Preferably, independent of the number of gNAs used to create the DSBs, in certain embodiments, each of the gNA is independently selected based on their proximity to the 5′ and 3′ ends of the template and target sequences, or the target location.

The selection and design of gNA can be performed using well-known principles or online tools, based on user input such as target genome and sequence type. In general, for Cas9, the gRNA is a short synthetic RNA composed of a “scaffold” sequence necessary for Cas9-binding and a user-defined 20 nucleotide “spacer” or “targeting” sequence which defines the genornic target to be bound or modified by the targeting sequence. For simplicity, “gRNA targets a Cas9 cleavage site” refers to the fact that the spacer or targeting sequence of the gRNA is designed to bind to a genomic target sequence and cleave it at the cleavage site.

Guide nucleic acids, including gRNAs and gDNAs according to the present disclosure may be anywhere from 10 nucleotides in length, including 10-50 nucleotides, 10-40, 10-30, 10-20, 15-25, 16-24, 17-23, 18-22, 19-21 and 20 nucleotides.

Preferably, the targeting sequence is sufficiently unique such that in theory it binds to a unique (compared to the rest of the genome) genomic target sequence. The target should be present immediately upstream (or 5′) of a Protospacer Adjacent Motif (or “PAM” sequence). The PAM sequence is absolutely necessary for target binding and the exact sequence is dependent upon the species of Cas9. In the most widely used Streptococcus pyogenes Cas9, the PAM sequence is 5′-NGG-3′ (“N” denotes any of the 4 standard nucleotides). Other PAM sequences for additional Cas9 in different species are known in the art. See exemplary PAM sequences listed in Table 4 below.

TABLE 4
PAM Sequences

| Species/Variant of Cas9 | PAM Sequence |
|---|---|
| Streptococcus pyogenes (SP); SpCas9 | NGG |
| SpCas9 D1135E variant | NGG (reduced NAG binding) |
| SpCas9 VRER variant | NGCG |
| SpCas9 EQR variant | NGAG |
| SpCas9 VQR variant | NGAN or NGNG |
| Staphylococcus aureus (SA); SaCas9 | NNGRRT or NNGRR(N) |
| Neisseria meningitidis (NM) | NNNNGATT |
| Streptococcus thermophilus (ST) | NNAGAAW |
| Treponema denticola (TD) | NAAAAC |

The Cas9-gRNA complex will bind any target genomic sequence with a PAM, but Cas9 only cleaves the target genomic sequence if sufficient homology exists between the gRNA spacer and target genomic sequence. The end result of Cas9-mediated DNA cleavage is a double strand break (DSB) within the target genomic sequence, at a cleavage site that is about 3-4 nucleotides upstream of the PAM sequence.

In some embodiments, double stranded breaks are generated at or on both sides of the target sequence. For example, in those embodiments where the target chromosome comprises a target location, such as a location into which a template sequence is to be inserted with little or no deletion of the target chromosome, then the double stranded break is generated at the target location. Exemplary target locations comprise cleavage sites for any of the nucleases described herein. As a further example, in those embodiments where the target chromosome comprises a target sequence, such as sequence that will be replaced or deleted by insertion of a template sequence, then double stranded breaks are generated on either side of the target sequence (i.e., both 5′ and 3′ of the target sequence).

In certain embodiments, the cleavage site of any selected endonuclease, for example a gNA targeting sequence, is within about 10 bp, about 20 bp, about 30 bp, about 50 bp, about 70, about 100 bp, about 200 bp, about 300 bp, about 400 bp, or about 500, of the target sequence or location.

In certain embodiments, the cleavage site of any selected endonuclease, for example a gNA targeting sequence, is within about 100 bp, about 200 bp, about 300 bp, about 400 bp, about 500 bp, about 600 bp, about 700 bp, about 800 bp, about 900 bp, about 1,000 bp, about 1,100 bp, about 1,200 bp, about 1,300 bp, about 1,400 bp, about 1,500 bp, about 1,600 bp, about 1,700 bp, about 1,800 bp, about 1,900 Or about 2,000, of the template sequence.

In some embodiments, the double stranded breaks are repaired by at least one DNA repair pathway is selected from the group consisting of resection, mismatch repair (MMR), nucleotide excision repair (NER), base excision repair (BER), canonical non-homologous end joining (canonical NHEJ), alternative non-homologous end joining (ALT-NHEJ), canonical homology directed-repair (canonical HDR), alternative homology directed repair (ALT-HDR), microhomology-mediated end joining (MMEJ), Blunt End Joining, Synthesis Dependent Microhomology Mediated End Joining, single strand annealing (SSA), Holliday junction model or double strand break repair (DSBR), synthesis-dependent strand annealing (SDSA), single strand break repair (SSBR), translesion synthesis repair (TLS), and interstrand crosslink repair (ICL), and DNA/RNA processing.

### Recovery of Engineered Chromosomes

The disclosure provides methods of recovering the engineered chromosomes described herein, and transferring said engineered chromosomes to a cellular environment suitable for downstream applications. In some embodiments, recovering the engineered chromosomes described herein comprises Micro-cell Mediated Chromosomal Transfer (MMCT).

Microcell-mediated chromosome transfer (MMCT) is a technique for fusing a microcell prepared from a donor cell with a recipient cell. By this technique, a particular (foreign) DNA (for example, chromosome) in the donor cell can be transferred into the recipient cell. The microcell is usually prepared by treating the donor cell with colcemid, although other methods may be used, and are envisaged within the scope of the instant disclosure.

An exemplary MMCT protocol comprises culturing the cell comprising the engineered chromosome in a cell culture medium comprising at least one micronucleus inducer under conditions sufficient to induce micronucleation thereby producing micronucleated cells, and collecting the micronucleated cells. Exemplary micronucleus inducers include, but are not limited to, microtubule polymerization inhibitors, a microtubule depolymerization inhibitors and spindle checkpoint inhibitors. Exemplary micronucleus inducers known in the art include, but are not limited to colcemid, colchicine, vincristine, or a combination thereof. For example, cells may be treated with 0.05 μg/mL to 0.25 μg/mL to induce micronucleation.

Micronucleated cells can be recovered using any suitable methods known in the art, including centrifugation and filtration.

Accordingly, the disclosure provides methods comprising recovering the engineered chromosome comprises exposing the cells to colcemid under conditions sufficient to induce micronucleation, and collecting micronucleated cells using centrifugation.

In some embodiments, the engineered chromosomes comprise one or more markers, for example the selectable or detectable markers introduced when engineering the chromosome with the template sequence. These markers can be used to follow the engineered chromosome, and select cells comprising the engineered chromosome following fusion with the micronucleated cells described supra.

Accordingly, the disclosure provides methods of generating an embryonic stem cell comprising: (a) fusing micronucleated cells comprising the engineered chromosome produced by the methods of the disclosure to ES cells, wherein (i) the ES cells comprise a chromosome homologous to the engineered chromosome, the homologous chromosome comprising a first fluorescent protein operably linked to a promoter capable of expressing the fluorescent protein in the ES cells, and (ii) at least a subset of the micronucleated cells comprise the engineered chromosome, and wherein the engineered chromosome comprises a second fluorescent protein different from the first fluorescent protein, the second fluorescent protein operably linked to a promoter capable of expressing the fluorescent protein in the ES cells; (b) selecting ES cells that express both the first and second fluorescent proteins; (c) culturing the ES cells selected in step (c) until the homologous chromosome is lost by at least a subset of the ES cells; and (d) selecting ES cells that express the second fluorescent protein and do not express the first fluorescent protein. In some embodiments, the ES cell is a mouse, rat, rabbit, guinea pig, hamster, sheep, goat, donkey, cow, horse, camel, chicken or monkey ES cell. In some embodiments, the ES cell is a, mouse ES cell. In some embodiments, the ES cell is a rat ES cell. In some embodiments, the ES cell is a monkey ES cell.

While the methods of generating an generating an embryonic stem cell described supra use two different fluorescent proteins as markers, the person of ordinary skill will appreciate that other markers can be suitable, as long as the markers on the engineered chromosome and the homologous chromosome are different. For example, two different selectable markers as described herein can be used, as well as two different surface molecules that can be recognized by labeled antibodies, or conjugated to selective marker such as a gold particle, which allows for selection via centrifugation. As a further example, in addition to fluorescent proteins as markers, puromycin and hygromycin/thymidine kinase (TK) marker can be used for positive-negative selection in this step. When thymidine kinase is expressed in the presence of particular thymidine analogues, these analogues are converted to toxic compounds which kill the cell. For example, a puromycin resistance marker and the hygromycin/TK marker are knocked into in the two chromosomes at the same location, and double positive single clones are selected by culturing in puromycin and hygromycin. After culturing for several days, puromycin and the thymidine kinase are used to select clones that have lost one copy of the chromosome, the chromosome bearing the hygromycin/TK marker.

In some embodiments, the methods of generating an embryonic stem cell comprise (a) fusing micronucleated cells comprising the engineered chromosome produced by the methods of the disclosure to ES cells, wherein (i) the ES cells comprise a chromosome homologous to the engineered chromosome, the homologous chromosome comprising a first marker, and (ii) at least a subset of the micronucleated cells comprise the engineered chromosome, and wherein the engineered chromosome comprises a second marker different from the first marker; (b) selecting ES cells that express both the first and second markers; (c) culturing the ES cells selected in step (c) until the homologous chromosome is lost by at least a subset of the ES cells; and (d) selecting ES cells that express the second marker and do not express the first marker.

Micronucleated cells can be fused to ES cells using any suitable methods. Fusion methods include, inter alia, electrofusion, viral induced fusion, and chemically induced fusion, for example through the addition of PEG1000 to the cells.

Given the inherent instability of the trisomy generated by the methods of recovering the engineered chromosome described above, culture the cells generated by fusion to the micronucleated cells for a period of at least 5 days, at least 7 days, at least 10 days, or at least 14 days may be sufficient to derive cells which have lost the homologous chromosome corresponding to the engineered chromosome. Alternatively, selection schemes employing negatively selectable markers, e.g. markers located on the homologous chromosome whose expression kills the cells when exposed to a selective regimen, may be employed. In some embodiments, selecting the cells at steps (b) and (d) comprises fluorescence activated cell sorting (FACS). For example, cells can be FACs sorted cells the express the second fluorescent protein used to mark the engineered chromosome, but not the first fluorescent protein used to mark the homologous chromosome.

### Cells

The disclosure provides cells for use in the methods of the disclosure. In some embodiments, the cells comprise embryonic stem (ES) cells, hybrid embryonic stem (EHS) cells, or zygotic cells. The disclosure also provides cells comprising the engineered chromosomes produced by the methods of the disclosure. The disclosure provides methods of isolating, fusion, and culturing the cells described herein.

Accordingly, the disclosure provides methods of fusing cells, to generate the EHS cells described herein. Cell fusion has been rendered possible through chemical, biological and physical means. Examples of these techniques include polyethylene glycol (PEG) fusion, fusagenic virus fusion and electrofusion, respectively.

The ES cells for use in the methods of the instant disclosure may be obtained from a variety of sources, and may be primary isolated ES cells or an artificially or naturally created ES cell line. The ES cells may also be first genetically modified to introduce useful traits such as expression of one or more markers, either prior to or after cell fusion to generate the EHS cells of the disclosure, or prior to or after the methods described herein

One commonly used technique is chemical fusion using, for example, PEG. This technology has been particularly successful in generating hybridomas. The fusion probability can be improved by exposure of the cells to intense electric fields for very brief periods, chemical agents can be used to effect linkage and proximation of cell pairs of the desired type (i.e. two types of EH cells), in a suspension prior to electric field exposure.

Electrofusion of cells involves bringing cells together in close proximity and exposing them to an alternating electric field. Under appropriate conditions, the cells are pushed together and there is a fusion of cell membranes and then the formation of fusate cells or hybrid cells.

Electrofusion of cells and apparatus for performing same are described in, for example, U.S. Pat. Nos. 4,441,972, 4,578,168 and 5,283,194, International Patent Application No. PCT/AU92/00473. Generally, the method involves selecting the cells and positioning them in a fluid-filled chamber adopted for use as a cell-fusing chamber. Individual pairs of cells may be involved in the fusion process, i.e. single cell fusion, or bulk fusion may occur with two populations each comprising two or more cells. Bulk fusion may be mini-bulk fusion where from about 2 to about 1000 cells are involved or macro-bulk fusion where greater than about 1000 cells are involved. Fusion may be facilitated by chemical means such as in the presence of PEG, biological means, such as in the presence of a fusagenic virus or by electrical means, i.e. electrofusion. The fusion may also involve a combination of these techniques. The cells may also be treated with a cytokine such as interleukin 3 (IL-3) to facilitate fusion.

Following cell fusion, a fused cell (fusate cell) or otherwise known as a hybrid cell is obtained comprising of nuclei of at least two cells encased in a fused lipid bilayer from the cells involved in the fusion. The nuclei of the cells fuse resulting in a hybrid cell with an abnormal number of chromosomes, which might be quadrupled or containing less or a greater number of chromosomes. The hybrid cell has the ability to divide and proliferate under appropriate culture conditions.

In some embodiments, EHS cells are generated through electrofusion. For example, human and mouse, human and rat, or human and monkey ES cells can be fused through electrofusion. In some embodiments, two EHS cells from two species selected from the group consisting of human, mouse, rat, rabbit, guinea pig, hamster, sheep, goat, donkey, cow, horse, camel, chicken and monkey undergo electrofusion to generate an EHS cell.

Generally, once fusion has occurred, the resulting hybrid cell is recovered in a suitable rich medium prior to being expanded in culture for use in in the methods of the disclosure. The recovery medium should contain factors allowing the recovery of the cell fusate following the stress of fusion. Such a supplement could include a high percentage of fetal calf serum, for example 20%.

The hybrid cells generated via cell fusion—may comprise unique cell surface markers which are useful in selecting these cells, monitoring fusion events.

In some embodiments, the cells of the disclosure comprise one or more genetic modifications, such as the introduction of a marker described herein. Genetic modifications can be carried out by any suitable means known in the art. For example, cells can be modified by transfection, transduction, electroporation, lipofection and the like.

Transfection as used herein refers to the introduction of nucleic acids, including naked or purified nucleic acids or vectors carrying a specific nucleic acid into cells, in particular eukaryotic cells, including mammalian cells. Any know transfection method can be employed in the context of the present disclosure. Some of these methods include enhancing the permeability of a biological membrane to bring the nucleic acids into the cell. Prominent examples are electroporation, microporation and lipofection. The methods may be used by themselves or can be supported by some, electromagnetic, and thermal energy, chemical permeation enhancers, pressure, and the like for selectively enhancing flux rate of nucleic acids into a host cell. Other transfection methods are also within the scope of the present disclosure, such as carrier-based transfection including lipofection or viruses (also referred to as transduction) and chemical based transfection. However, any method that brings a nucleic acid inside a cell can be used. A transiently-transfected cell will carry/express transfected RNA/DNA for a short amount of time and not pass it on. A stably-transfected cell will continuously express transfected DNA and pass it on: the exogenous nucleic acid has integrated into the genome of a cell.

A number of viruses have been used as gene transfer vectors or as the basis for preparing gene transfer vectors, including papovaviruses, adenovirus, vaccinia virus, adeno-associated virus, lentiviruses, Sindbis and Semliki Forest virus and retroviruses of avian and human origin.

Chemical techniques of gene transfer including calcium phosphate co-precipitation, mechanical techniques, for example, microinjection, membrane fusion-mediated transfer via liposomes and direct DNA uptake and receptor-mediated DNA transfer. Viral-mediated gene transfer can be combined with direct in vivo gene transfer using liposome delivery, allowing one to direct the viral vectors to particular cells. Alternatively, the retroviral vector producer cell line can be injected into particular tissue. Injection of producer cells would then provide a continuous source of vector particles.

The disclosure provides methods of culturing the cells of the disclosure. Many stem cell media culture or growth environments are envisioned in the embodiments described herein, including defined media, conditioned media, feeder-free media, serum-free media and the like. As used herein, the term “growth environment” equivalents thereof is an environment in which undifferentiated or differentiated stem cells (e.g., embryonic stem cells) will proliferate in vitro. Features of the environment include the medium in which the cells are cultured, and a supporting structure (such as a substrate on a solid surface) if present. Methods for culturing or maintaining cells are also described in PCT/US2007/062755; U.S. application Ser. No. 11/993,399, and U.S. application Ser. No. 11/875,057.

Base cell culture media are known in the art and are commercially available. Exemplary base cell culture media include, but are not limited to, DMEM, CMRL or RPMI based media.

The cell culture media used in the cell culture methods of the instant disclosure can include serum, or be serum-free. Cell culture medium can also include one or more supplements or other media components known in the art, such as B27 supplement, insulin, glucose, growth factors such as EGF and FGF, and cytokines.

The term “feeder cell” refers to a culture of cells that grows in vitro and secretes at least one factor into the culture medium, and that can be used to support the growth of another cell of interest in culture. As used herein, a “feeder cell layer” can be used interchangeably with the term “feeder cell.” A feeder cell can comprise a monolayer, where the feeder cells cover the surface of the culture dish with a complete layer before growing on top of each other, or can comprise clusters of cells. In a preferred embodiment, the feeder cell comprises an adherent monolayer.

Similarly, embodiments in which ES or EHS cell cultures or aggregate suspension cultures are grown in defined conditions or culture systems without the use of feeder cells are “feeder-free”. Feeder-free methods are also described in U.S. Pat. No. 6,800,480. In some embodiments, ES or ESH cell can be cultured in a two or three dimensional environment. In the U.S. Pat. No. 6,800,480, extracellular matrix is prepared by culturing fibroblasts, lysing the fibroblasts in situ, and then washing what remains after lysis. Alternatively, in U.S. Pat. No. 6,800,480 extracellular matrix can also be prepared from an isolated matrix component or a combination of components selected from collagen, placental matrix, fibronectin, laminin, merosin, tenascin, heparin sulfate, chondroitin sulfate, dermatan sulfate, aggrecan, biglycan, thrombospondin, vitronectin, and decorin.

In some embodiments, culturing methods or culturing systems are free of animal sourced products. In other embodiments the culturing methods are xeno-free.

The disclosure contemplates differentiating the ES cells comprising the engineered chromosomes described herein into different cell types for use in various downstream applications. ES cells can be induced to differentiate into a variety of cell types in vitro using a variety of strategies, usually involving supplementing the cell culture medium with exogenous biochemical compositions that direct recapitulate endogenous developmental cell signals and direct cell specific differentiation, strategies for differentiating ES cells are discussed in Vazin and Freed, Restor Neurol Neurosci (2010) 28(4): 589-603, the contents of which are incorporated by reference herein.

For example, the population of ES or EHS cells can be further cultured in the presence of certain supplemental growth factors to obtain a population of cells that are or will develop into different cellular lineages, or can be selectively reversed in order to be able to develop into different cellular lineages. The term “supplemental growth factor” is used in its broadest context and refers to a substance that is effective to promote the growth of an ES cell, maintain the survival of a cell, stimulate the differentiation of a cell, and/or stimulate reversal of the differentiation of a cell. Further, a supplemental growth factor may be a substance that is secreted by a feeder cell into its media. Such substances include, but are not limited to, cytokines, chemokines, small molecules, neutralizing antibodies, and proteins. Growth factors may also include intercellular signaling polypeptides, which control the development and maintenance of cells as well as the form and function of tissues. In preferred embodiments, the supplemental growth factor is selected from the group comprising steel cell factor (SCF), oncostatin M (OSM), ciliary neurotrophic factor (CNTF), Interleukin-6 (IL-6) in combination with soluble Interleukin-6 Receptor (IL-6R), a fibroblast growth factor (FGF), a bone morphogenetic protein (BMP), tumor necrosis factor (TNT), and granulocyte macrophage colony stimulating factor (GMN-CSF).

The progression of stem cells to various multipotent and/or differentiated cells can be monitored by determining the relative expression of genes, or gene markers, characteristic of a specific cell type, as compared to the expression of a second or control gene, e.g., housekeeping genes. In some processes, the expression of certain markers is determined by detecting the presence or absence of the marker. Alternatively, the expression of certain markers can be determined by measuring the level at which the marker is present in the cells of the cell culture or cell population. In such processes, the measurement of marker expression can be qualitative or quantitative. One method of quantitating the expression of markers that are produced by marker genes is through the use of quantitative PCR (Q-PCR). Methods of performing Q-PCR are well known in the art. Other methods which are known in the art can also be used to quantitate marker gene expression. For example, the expression of a marker gene product can be detected by using antibodies specific for the marker gene product of interest.

### Transgenic Animals

The disclosure provides transgenic animals, for example transgenic mice, comprising the engineered chromosomes of the disclosure, and methods of making same.

Selection of suitable methods for making transgenic animals from the ES cells or zygotic cells comprising the engineered chromosomes described herein will depend on the animal, and will be known to persons of skill in the art.

In exemplary methods, ES cells comprising the engineered chromosome incorporated into an embryo at the blastocyst stage of development, which is then implanted in a pregnant or pseudopregnant female and carried to term. The result is a chimeric animal. If the ES cells give rise to germ cells, the progeny of the animal will be fully transgenic, and carry the engineered chromosome.

In some embodiments the transgenic animal is a mouse, rat, rabbit, guinea pig, hamster, sheep, goat, donkey, cow, horse, camel, chicken or monkey.

In some embodiments, the transgenic animal is a mouse. In some embodiments, producing the transgenic mouse comprises injecting the ES cell into a diploid blastocyst, nuclear transfer from the ES cell to an enucleated mouse embryo, or tetraploid embryo complementation.

In some embodiments, the method further comprises transferring the ES cell or the zygote into a pseudo-pregnant female. In mice, pseudopregnant females are readied by mating six- to eight-week-old female mice in natural estrus with vasectomized males. Zygotes processed for same day transfer to pseudopregnant females can be removed from culture and placed into a pre-warmed suitable medium (such as M2 medium) and transferred via the oviduct into 0.5 days post coitum pseudopregnant females (e.g. age 9-11 weeks).

Once the engineered chromosome inserted into a host mammal using the methods of the disclosure, presence of the engineered chromosome can be verified in the resulting transgenic animal (e.g., mouse) or progeny thereof. Such verification typically includes one or more of genotyping animals that potentially carry the engineered chromosome, polymerase chain reaction amplification of junctional sequences, direct sequencing of certain stretches of DNA (e.g., the template sequence), and genetic mapping. Such techniques are well-known in the art.

The disclosure provides transgenic mouse comprising the engineered chromosomes of the disclosure. In some embodiments, the transgenic mouse comprises one or more genes that have been humanized, for example any one of the genes described in Tables 1 and 2. In some embodiments, the animal model comprises more than one humanized gene (for example 1, 2, 5, 10, 20, 50, 100 or more genes). In some embodiments, the transgenic mouse comprises all or part of an immunoglobulin gene that has been humanized. In some embodiments, the transgenic mouse comprises all or part of TCR subunit gene that has been humanized.

In some embodiments of the transgenic mice of the disclosure, mouse chromosome 12 comprises a sequence of a human IGH variable region in place of a mouse Igh variable region. In some embodiments, the mouse Igh variable region comprises V_{H}, D_{H} and J_{H}1-6 gene segments and intervening non-coding sequences. In some embodiments, the human IGH variable region comprises V_{H}, D_{H} and J_{H}1-6 gene segments and intervening non-coding sequences. In some embodiments, the engineered chromosome is a mouse chromosome 6 comprising a sequence of a human IGK variable region in place of a mouse Igk variable region. In some embodiments, the mouse Igk variable region sequence comprises a sequence encoding mouse Vₖ, and Jₖ₁₋₅ gene segments and intervening non-coding sequences. In some embodiments, the template sequence comprises a human IGK variable region sequence. In some embodiments, the human IGK variable region sequence comprises a sequence encoding human Vₖ, and Jₖ₁₋₅ gene segments and intervening non-coding sequences.

### Applications

Downstream applications of the cells and transgenic animals comprising the engineered chromosomes described herein are contemplated as within the scope of the instant disclosure.

Exemplary downstream applications include basic and applied research into animal models of human diseases and disorders using an animal model (e.g., mouse, rat or monkey) that has been humanized for one or more human genes. Exemplary, but non limiting, genes that can be humanized by replacement of the model animal homolog with the human homology are described in Tables 1 and 2. Animal models for human diseases associated with chromosomal aberrations (translocations, inversions and the like), can also be made using the methods described herein. Any animal models that need large scale chromosomal rearrangements for fragments larger than 300 kB, such as, for example, a Duchenne Muscular Dystrophy (DMD) humanized mouse disease model, or that require the large-scale insertion or replacement of arrays of up to hundreds of genes are envisaged as within the scope of the instant disclosure.

In some embodiments, for example those embodiments where the Igh variable regions of the animal have been humanized, transgenic animals of the disclosure can be used to produce humanized antibodies. For example, such animals can produce specific B cells with human, or humanized, antibodies. In some embodiments, for example those embodiments where the Igk or Igl variable regions of the animal have been humanized, transgenic animals of the disclosure can be used to produce humanized antibodies.

In some embodiments, for example those embodiments where a template sequence comprising an antibody or an antigen fragment thereof has been inserted into the target chromosome, the transgenic animals of the disclosure can be used to generate an antibody or antigen binding fragment. For example, transgenic animals can be used to generate single chain variable fragments (scFv), nanobodies, dual-specific antibodies, and multi-specific antibodies, among others. Such antibodies could be used for research or therapeutic purposes.

Exemplary downstream applications include applications where the engineered chromosomes are not incorporated into a transgenic animal. Instead, as one example, ES cells comprising the engineered chromosomes are differentiated into another cell type, which can be used for research or therapeutic purposes.

### Kits

The disclosure provides kits comprising the nucleic acid molecules described herein. In some embodiments, the nucleic acid molecules are vectors, such as plasmids.

In some embodiments of the kits of the disclosure, the kits comprise cells for use in the methods described herein, for example EHS cells that have been cryopreserved. In some embodiments, the kits comprise instructions for use of the nucleic acid molecules, and optionally cells.

### EXAMPLES

### Example 1: Establishment of Embryonic Hybrid Stem (EHS) Cells

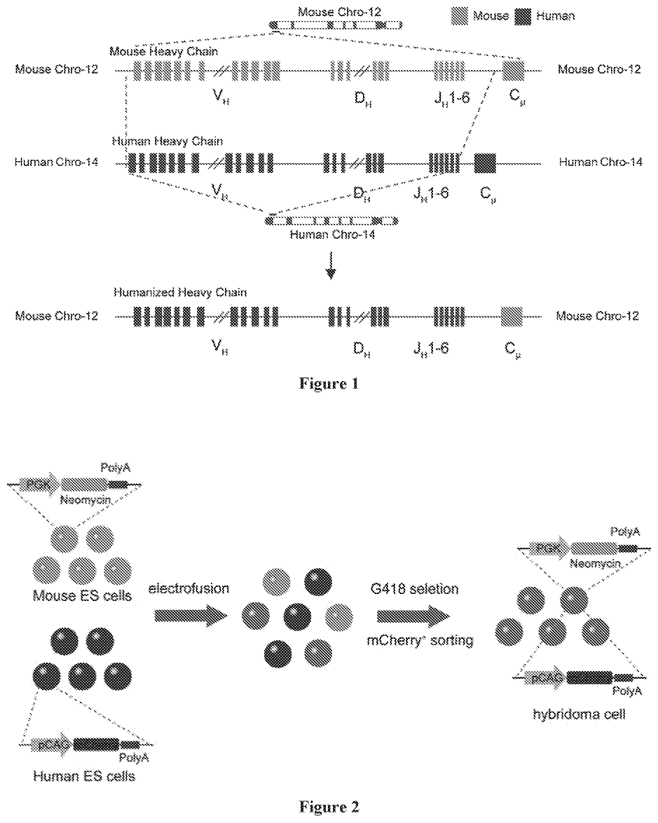

The overall goal of this study was to obtain mice humanized for the variable domains of the Igh and Igk genes. Human and mice show high similarity in arrangement and expression of antibody genes, and the genomic organizations of the heavy chain are also similar in humans and mice. Therefore, a humanized version of the mouse Igh or Igk gene variable domains could be obtained by replacing ˜3 MB mouse genomic sequences containing all the V_{H}, D_{H}, and J_{H} gene segments with roughly 1 MB of contiguous human genomic sequence containing the equivalent human gene fragments (FIG. 1).

The first step towards creating a humanized mouse Igh gene was to create a mouse embryonic hybrid stem (EHS) cell by fusing a mouse embryonic stem (ES) cell to a human ES cell, to create a cell with both mouse and human Igh genes.

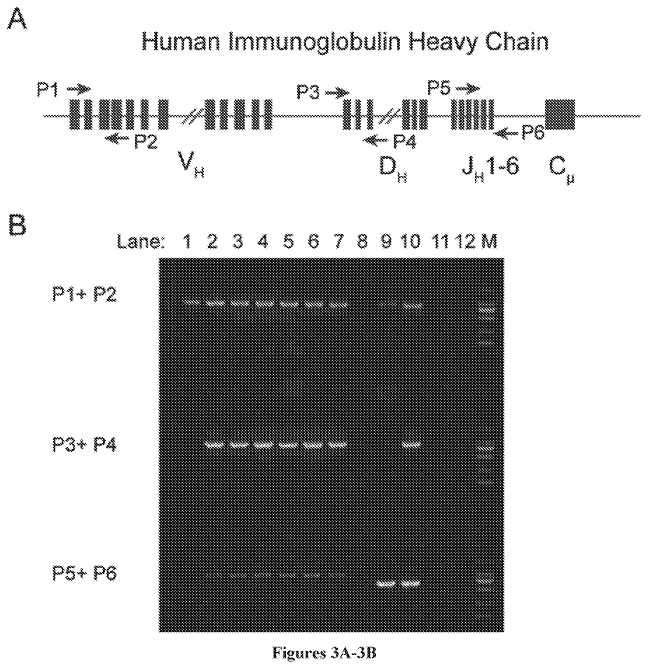

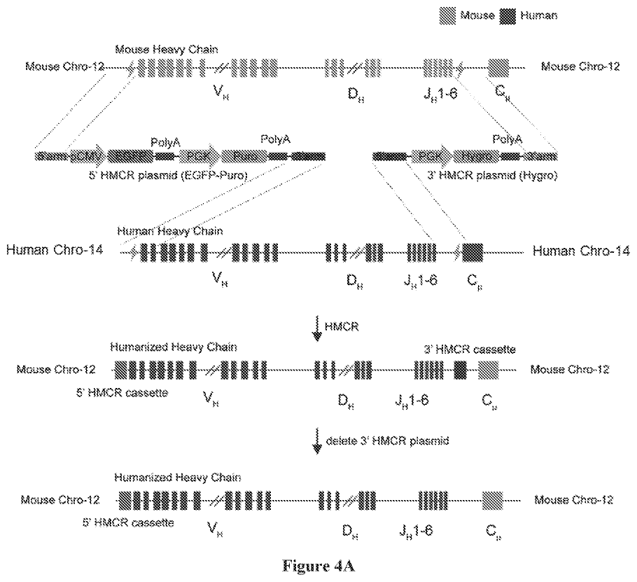

Engineered mouse cells expressing a neomycin resistance gene under the control of a PGK promoter, and engineered human ES cells expressing an mCherry marker under control of the CAG promoter, were fused by electrofusion, according to standard methods supplied by the manufacturer of the electrofusion instrument. Hybrid EHS cells were cultured in mouse ES cell medium containing G418 for 7 days, and surviving cells were sorted by fluorescence activated cell sorting (FACS) according to the expression level of mCherry (FIG. 2). Positive cells were continuously cultured in mouse ES cell medium containing G418, and single cell clones were isolated into separate wells for growth. Next, genomic DNA was extracted for each single cell clone for genotyping. Specifically, three pairs of primers for the V,D, J regions of human immunoglobulin heavy (IGH) chain (FIG. 3A) were used to perform PCR to confirm the presence of the targeted sequences (FIG. 3B) in the EHS clones. Only clones with all the three desired regions were retained for further experiments.

### Example 2: Engineering a Humanized Chromosome

2.1. EHC establishment by HDR-Mediated Chromosome Rearrangement (HMCR)

To obtain mouse embryonic hybrid stem (EHS) cells humanized for their variable domains of the Igh gene, the ˜3 MB variable domains of Igh gene on mouse chromosome 12 were replaced with ˜1 MB variable domains of the human IGH gene on human chromosome 14 by HDR-Mediated Chromosome Rearrangement (HMCR; FIG. 4A).

Two plasmids were designed to mediate the HMCR process, and are shown in FIG. 4A.

The 5′ HMCR plasmid was designed to mediate the replacement of 5′ end of the mouse Igh gene with its human counterpart, and the 3′ HMCR plasmid mediated the replacement of 3′ end of the mouse Igh gene with its human counterpart. The 5′ HMCR plasmid contained a 5′ arm homologous to the 5′ end of the mouse Igh gene, a 3′ arm homologous to the 5′ of the human IGH gene, and a cassette of CMV-EGFP-polyA-PGK-Puromycin-poly, which was inserted between the two homology arms. Similarly, the 3′ HMCR plasmid contained a 5′ arm homologous to the 3′ of human IGH variable loci, a 3′ arm homologous to the 3′ of mouse Igh variable loci and a PGK-Hygromycin-polyA cassette inserted between the two homology arms (see FIG. 4A). Homology arms were between 600 bp and 1000 bp in length. At the same time, four plasmids containing Cas9 and sgRNAs targeting the 5′ and 3′ ends of the Igh variable domains in mouse and human were also designed (see zigzag marks in FIG. 4A, sgRNA targeting sequences provided in Table 7). These six plasmids were co-transfected as circular plasmids into the EHS cells obtained in Example 1 using standard methods, and the resulting cells were cultured in mouse ES cell medium containing Puromycin and Hygromycin for 7 days. Surviving GFP-positive single clones were picked for further culturing.

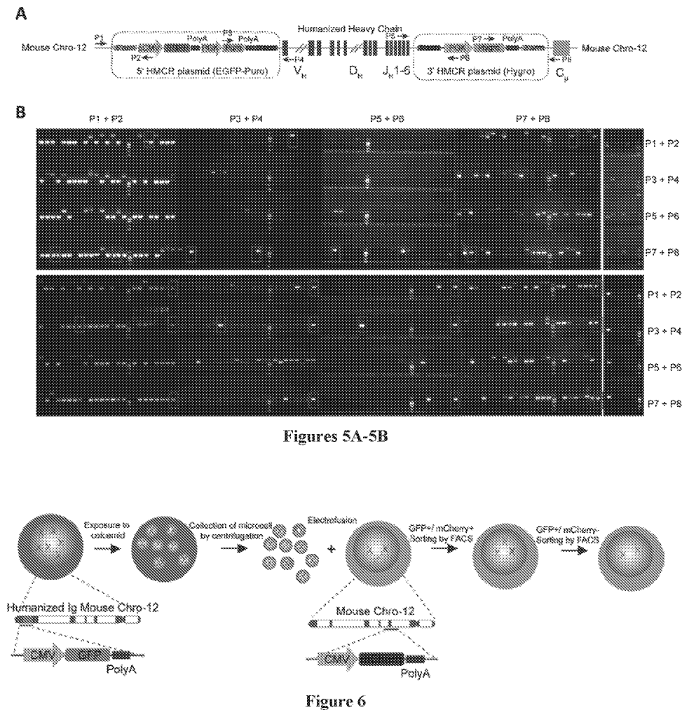

Genotyping was performed to identify the desired single clones with successful HMCR. For genotyping, four pairs of PCR primers were designed as shown in FIG. 5A. For the first pair of primers, the forward primer was designed upstream of the 5′ homology arm of the mouse Igh 5′ HMCR plasmid, and the reverse primer was within the CMV promoter region (FIG. 5A). For the second pair of primers, the forward primer was within the Puromycin gene of the 5′ HMCR plasmid, and the reverse primer was downstream of 5′ homologous arm of human IGH, within the human IGH sequence (FIG. 5A). For the third pair of primers, the forward primer was upstream of the 3′ homologous arm of human IGH variable region, and the reverse primer was in the PGK promoter region of the 3′ HMCR plasmid (FIG. 5A). For the last pair of primers, the forward primer was in the Hygromycin gene of the 3′ HMCR plasmid, and the reverse primer was downstream of 3′ homologous of the 3′ HMCR plasmid, within the mouse Igh variable domain (FIG. 5A). PCR amplification was performed with each primer pair for each clone, and only clones showing positive PCR products for all four genotyping tests were retained for further experiments. Out of 196 isolated clones in this step, 6 were identified as positive for all four PCR amplicons (FIG. 5B).

To facilitate the expression of human IGH gene in the EHS cells with successful HMCR, the 3′ selection marker was deleted from the genome of positive clones by homology directed repair (HDR) (FIG. 4A), although non-homologous end joining (NHEJ), microhomology-mediated end joining (NMEJ) and homology-mediated ed joining (HMEJ) methods could also be used. The process described above successfully established an engineered humanized chromosome (EHC) which had the variable domain encompassing the V_{H}, D_{H}, and J_{H}1-6 gene segments of the mouse Igh gene on mouse chromosome 12 replaced with the equivalent human regions by HMCR in Et-S cells.

Sequences of the plasmids used to mediate the HMCR process are provided in Tables 5 and 6 below.

TABLE 5
Exemplary 5′ plasmid sequences for HMCR mediated replacement of mouse Igh
variable region with corresponding human region

| Name | Sequence |
|---|---|
| 5′ homology arm | ctgaagtcagattgggcaacttcatagtatacaatagaaaatctacctgcagatgagttcagaaccagc |
| (mouse), | agggggcacaatggggccaagaatccctagcagagagatgtggtgtgtgtgcaggggactctgcat |
| coordinates | cctctgtggtttcctttcttaacttacatgtacctgtagtgattgacatgtaacgtttccacgctcaaacactg |
| 115973952- | tgaagatactttgctaaacacttcaaagatttatgttttcttgatgtgtgcatgtgtgtattcttttttgttttta |
| 115974751, | gacacagggtttctctgtgtagtcctggctgccctggaactcactctgtagaccaggctggcctcgaactca |
| of chromosome 12, | gaaatctgcctgcttctgcctcccaagtgctgaagttaaagacatgtgccaccattgcctggccatgtgt |
| GRCm39 | gtattcttgatgcactcttctgttgacagatacacagtttatttccataatttatttattgtgatggtgctgcaat |
| (SEQ ID NO: 1) | aatcacttatgtacaaatgtttctgaagtatatttagttttggtcatttgggtgattatttttttctttctagtat |
| | atagcattttggaaaggtagatattaattgtatgtatgggaaggaggctgtaaattctaataacttagctgctttt |
| | gaaatttgtcctcaattctatcatccttgtaaccaccttaaatccatctattagccttgtcacaagtgagcca |
| | ctgtctcaggctgcaaatctttttatagattaggtcgtgatgttacatccacagcctctgcacaatgctcag |
| pCMV-GFP, GFP | atagtaatcaattacggggtcattagttcatagcccatatatggagttccgcgttacataacttacggtaaa |
| is underlined | tggcccgcctggctgaccgcccaacgacccccgcccattgacgtcaataatgacgtatgttcccatag |
| (SEQ ID NO: 2) | taacgccaatagggactttccattgacgtcaatgggtggagtatttacggtaaactgcccacttggcagt |
| | acatcaagtgtatcatatgccaagtccgccccctattgacgtcaatgacggtaaatggcccgcctggca |
| | ttatgcccagtacatgaccttacgggactttcctacttggcagtacatctacgtattagtcatcgctattacc |
| | atggtgatgcggttttggcagtacaccaatgggcgtggatagcggtttgactcacggggatttccaagt |
| | ctccaccccattgacgtcaatgggagtttgttttggcaccaaaatcaacgggactttccaaaatgtcgta |
| | ataaccccgccccgttgacgcaaatgggcggtaggcgtgtacggtgggaggtctatataagcagagg |
| | tcgtttagtgaaccgtcagatcAcgcgtgccaccatgctcagcaagggcgaggagctgttcaccgg |
| | ggtggtccccatcctggtccagctggacggcgacgtaaacggccacaagttcagcgtctcccccga |
| | gggcgagggcgatgccacctacggcaagctgaccctgaagttcatctgcaccaccggcaagctgcc |
| | cgtgccctcgcccaccctcgtgaccaccctgacctacggcgtccagtgcttcagccgctaccccgac |
| | cacatgaagcagcaccacttcttcaagtcccccatgcccgaaggctacgtccaggagcgcaccatctt |
| | cttcaaggacgacggcaactacaagacccccgcccagctgaagttcgaggccgacaccctggtga |
| | accgcatcgagctgaagggcatcgacttcaaggaggaccgcaacatcctggggcacaagctggagt |
| | acaactacaacagccacaacgtctatatcatggccgacaagcagaagaacggcatcaaggtgaactt |
| | caagatccgccacaacatcgaggacggcagcctgcagctgccgaccactaccagcagaacaccc |
| | ccatcggcgacggccccctgctcctgcccgacaaccactacctgagcacccagtccgccctgagca |
| | aagaccccaacgagaagcgcgatcacatggtcctcctggagttcgtcacccccgcccggatcactct |
| | cggcatggacgagctgtacaagtaa |
| PGK-Puromycin N- | gggttggggttgcgccttttccaaggcagccctgggtttgcgcagggacgcggctgctctgggcgtg |
| acetyltransferase | gttccgggaaacgcagcggcgccgaccctgggactcgcacattcttcacgtccgttcgcagcgtcac |
| (SEQ ID NO: 3) | ccggatcttcgcgctacccttgtgggccccccggcgacgcttcctgctccgcccctaagtcgggaag |
| | gttccttgcggttcgcggcgtgccggacgtgacaaacggaagccgcacgtctcactagtaccctcgc |
| | agacggacagcgccagggagcaatggcagcgcgccgaccgcgatgggctgtggccaatagcggc |
| | tgctcagcagggcgcgccgagagcagcggccgggaaggggcggtgcgggaggcggggtgtgg |
| | ggcggtagtgtgggccctgttcctgcccgcgcggtgttccgcattctgcaagcctccggagcgcacgt |
| | cggcagtcggctccctcgttgaccgaatcaccgacctctctccccagggggatccaccggagcttac |
| | catgaccgagtacaagcccacggtgcgcctcgccacccgcgacgacgtccccagggccgtacgca |
| | ccctcgccgccgcgttcgccgactaccccgccacgcgccacaccgtcgatccggaccgccacatcg |
| | agcgggtcaccgagctgcaagaactcttcctcacgcgcgtcgggctcgacatcggcaaggtgtgggt |
| | cgcggacgacggcgccgcggtggcggtctggaccacgccggagagcgtcgaagcgggggcggt |
| | gttcgccgagatcggcccgcgcatggccgagttgagcggttcccggctggccgcgcagcaacagat |
| | ggaaggcctcctggcgccgcaccggcccaaggagcccgcgtggttcctggccaccgtcggcgtctc |
| | gcccgaccaccagggcaagggtctgggcagcgccgtcgtgctccccggagtggaggcggccgag |
| | cgcgccggggtgcccgccttcctggaaacctccgcgccccgcaacctccccttctacgagcggctcg |
| | gcttcaccgtcaccgccgacgtcgaggtgcccgaaggaccgcgcacctggtgcatgacccgcaagc |
| | ccggtgcctgacgc |
| | agtaggagacatgcaaatagggccctcactctgctgaagaaaaccagccctgcagctctgggagag |
| 3′ homology arm | gagccccagccctgggattcccagctgtttctgcttgctgatcaggactgcacacagagaactcaccat |
| (human), | ggagtttgggctgagctgggttttccttgttgctattttaaaaggtgattcatggagaactggagatatgg |
| coordinates | agtgtgaatggacatgagtgagataagcagtggatgtgtgtggcagtttctgaccagggtgtctctgtgt |
| 106810109- | ttgcaggtgtccagtgtgaggtgcagctggtggagtccgggggaggcttagttcagcctggggggtc |
| 106811028 of | cctgagactctcctgtgcagcctctggattcaccttcagtagctactggatgcactgggtccgccaagct |
| chromosome 14, | ccagggaaggggctggtgtgggtctcacgtattaatagtgatgggagtagcacaagctacgcggact |
| GRCh38.p13 | ccgtgaagggccgattcaccatctccagagacaacgccaagaacacgctgtatctgcaaatgaacag |
| (SEQ ID NO: 4) | tctgagagccgaggacacggctgtgtattactgtgcaagagacacagtgaggggaagtcaatgtgag |
| | cccagacacaaacctcgctgcaggggcatctgagaccacgagggggtgtcctgggccctgtgaact |
| | gggctgctctccgtggcagcggctggtggtgctaaaggctgattttctctcagcatctggggctgattca |
| | tcaagtttcctcagagaacctttcagatttacaattctgtacttacgtttaatgtcttgaatgtgacactttcc |
| | ttccctggtgtgtctttgtttttgtgacaagaggacacattctcacctccacagaagcccgagtgtcacttt |
| | ggggacagaaatgaccctgccct |
| Full length (from | ctgaagtcagattgggcaacttcatagtatacaatagaaaatctacctgcagatgagttcagaaccagc |
| homology arm to | agggggcacaatggggccaagaatccctagcagagagatgtggtgtgtgtgcaggggactctgcat |
| homology arm) | cctctgtggtttcctttcttaacttacatgtacctgtagtgattgacatgtaacgtttccacgctcaaacactg |
| (SEQ ID NO: 5) | tgaagatactttgctaaacacttcaaagatttatgttttcttgatgtgtgcatgtgtgtattcttttttgttttta |
| | gacacagggtttctctgtgtagtcctggctgccctggaactcactctgtagaccaggctggcctcgaactca |
| | gaaatctgcctgcttctgcctcccaagtgctgaagttaaagacatgtgccaccattgcctggccatgtgt |
| | gtattcttgatgcactcttctgttgacagatacacagtttatttccataatttatttattgtgatggtgctgcaat |
| | aatcacttatgtacaaatgtttctgaagtatatttagttttggtcatttgggtgattatttttttctttctagtat |
| | gcattttggaaaggtagatattaattgtatgtatgggaaggaggctgtaaattctaataacttagctgctttt |
| | gaaatttgtcctcaattctatcatccttgtaaccaccttaaatccatctattagccttgtcacaagtgagcca |
| | ctgtctcaggctgcaaatctttttatagattaggtcgtgatgttacatccacagcctctgcacaatgctcag |
| | ttaattaaatagtaatcaattacggggtcattagttcatagcccatatatggagttccgcgttacataactta |
| | cggtaaatggcccgcctggctgaccgcccaacgacccccgcccattgacgtcaataatgacgtatgtt |
| | cccatagtaacgccaatagggactttccattgacgtcaatgggtggagtatttacggtaaactgcccact |
| | tggcagtacatcaagtgtatcatatgccaagtccgccccctattgacgtcaatgacggtaaatggcccg |
| | cctggcattatgcccagtacatgaccttacgggactttcctacttggcagtacatctacgtattagtcatcg |
| | ctattaccatggtgatgcggttttggcagtacaccaatgggcgtggatagcggtttgactcacggggatt |
| | tccaagtctccaccccattgacgtcaatgggagtttgttttggcaccaaaatcaacgggactttccaaaat |
| | gtcgtaataaccccgccccgttgacgcaaatgggggtaggcgtgtacggtgggaggtctatataag |
| | cagaggtcgtttagtgaaccgtcagatcacgcgtgccaccatggtgagcaagggcgaggagctgttc |
| | accggggtggtgcccatcctggtcgagctggacggcgacgtaaacggccacaagttcagcgtgtcc |
| | ggcgagggcgagggcgatgccacctacggcaagctgaccctgaagttcatctgcaccaccggcaa |
| | gctgcccgtgccctggcccaccctcgtgaccaccctgacctacggcgtgcagtgcttcagccgctac |
| | cccgaccacatgaagcagcacgacttcttcaagtccgccatgcccgaaggctacgtccaggagcgc |
| | accatcttcttcaaggacgacggcaactacaagacccgcgccgaggtgaagttcgagggcgacacc |
| | ctggtgaaccgcatcgagctgaagggcatcgacttcaaggaggacggcaacatcctggggcacaag |
| | ctggagtacaactacaacagccacaacgtctatatcatggccgacaagcagaagaacggcatcaagg |
| | tgaacttcaagatccgccacaacatcgaggacggcagcgtgcagctcgccgaccactaccagcaga |
| | acacccccatcggcgacggccccgtgctgctgcccgacaaccactacctgagcacccagtccgccc |
| | tgagcaaagaccccaacgagaagcgcgatcacatggtcctgctggagttcgtgaccgccgccggga |
| | tcactctcggcatggacgagctgtacaagtaactgtgccttctagttgccagccatctgttgtttgcccct |
| | cccccgtgccttccttgaccctggaaggtgccactcccactgtcctttcctaataaaatgaggaaattgc |
| | atcgcattgtctgagtaggtgtcattctattctggggggtggggggggcaggacagcaagggggag |
| | gattgggaagacaatagcaggcatgctggggatgcggtgggctctatggcggcgcgccggggttgg |
| | ggttgcgccttttccaaggcagccctgggtttgcgcagggacgcggctgctctgggcgtggttccggg |
| | aaacgcagcggcgccgaccctgggactcgcacattcttcacgtccgttcgcagcgtcacccggatctt |
| | cgccgctacccttgtgggccccccggcgacgcttcctgctccgcccctaagtcgggaaggttccttgc |
| | ggttcgcggcgtgccggacgtgacaaacggaagccgcacgtctcactagtaccctcgcagacggac |
| | agcgccagggagcaatggcagcgcgccgaccgcgatgggctgtggccaatagcggctgctcagca |
| | gggcgcgccgagagcagcggccgggaaggggcggtgcgggaggggggtgtggggcggtagt |
| | gtgggccctgttcctgcccgcgcggtgttccgcattctgcaagcctccggagcgcacgtcggcagtc |
| | ggctccctcgttgaccgaatcaccgacctctctccccagggggatccaccggagcttaccatgaccga |
| | gtacaagcccacggtgcgcctcgccacccgcgacgacgtccccagggccgtacgcaccctcgccg |
| | ccgcgttcgccgactaccccgccacgcgccacaccgtcgatccggaccgccacatcgagcgggtca |
| | ccgagctgcaagaactcttcctcacgcgcgtcgggctcgacatcggcaaggtgtgggtcgcggacg |
| | acggcgccgcggtggcggtctggaccacgccggagagcgtcgaagcgggggcggtgttcgccga |
| | gatcggcccgcgcatggccgagttgagcggttcccggctggccgcgcagcaacagatggaaggcc |
| | tcctggcgccgcaccggcccaaggagcccgcgtggttcctggccaccgtcggcgtctcgcccgacc |
| | accagggcaagggtctgggcagcgccgtcgtgctccccggagtggaggcggccgagcgcgccgg |
| | ggtgcccgccttcctggaaacctccgcgccccgcaacctccccttctacgagcggctcggcttcaccg |
| | tcaccgccgacgtcgaggtgcccgaaggaccgcgcacctggtgcatgacccgcaagcccggtgcc |
| | tgacgcaagtaactgtgccttctagttgccagccatctgttgtttgcccctcccccgtgccttccttgaccc |
| | tggaaggtgccactcccactgtcctttcctaataaaatgaggaaattgcatcgcattgtctgagtaggtgt |
| | cattctattctggggggtggggtggggcaggacagcaagggggaggattgggaagacaatagcag |
| | gcatgctggggatgcggtgggctctatggcagtaggagacatgcaaatagggccctcactctgctga |
| | agaaaaccagccctgcagctctgggagaggagccccagccctgggattcccagctgtttctgcttgct |
| | gatcaggactgcacacagagaactcaccatggagtttgggctgagctgggttttccttgttgctattttaa |
| | aaggtgattcatggagaactggagatatggagtgtgaatggacatgagtgagataagcagtggatgtg |
| | tgtggcagtttctgaccagggtgtctctgtgtttgcaggtgtccagtgtgaggtgcagctggtggagtcc |
| | gggggaggcttagttcagcctggggggtccctgagactctcctgtgcagcctctggattcaccttcagt |
| | agctactggatgcactgggtccgccaagctccagggaaggggctggtgtgggtctcacgtattaatag |
| | tgatgggagtagcacaagctacgcggactccgtgaagggccgattcaccatctccagagacaacgc |
| | caagaacacgctgtatctgcaaatgaacagtctgagagccgaggacacggctgtgtattactgtgcaa |
| | gagacacagtgaggggaagtcaatgtgagcccagacacaaacctcgctgcaggggcatctgagac |
| | cacgagggggtgtcctgggccctgtgaactgggctgctctccgtggcagcggctggtggtgctaaag |
| | gctgattttctctcagcatctggggctgattcatcaagtttcctcagagaacctttcagatttacaattctgta |
| | cttacgtttaatgtctctgaatgtgacactttccttccctggtgtgtctttgtttttgtgacaagaggacacatt |
| | ctcacctccacagaagcccgagtgtcactttggggacagaaatgaccctgccct |

TABLE 6
Exemplary 3′ plasmid sequences for HMCR mediated replacement of mouse Igh
variable region with corresponding human region

| Name | Sequence |
|---|---|
| 5′ homology arm | gccagggtctcagggtcagagtcttggaggcattttggaggtcaggaaagaaagctggggagaggg |
| (human), | acccttcgaatgggaacccagcctgtcctccccaagtccggccacagatgtcggcagctggggggct |
| coordinates | ccttcggctggtctggggtgacctctctccgcttcacctggagcattctcaggggctgtcgtgatgattg |
| 105862994- | cgtggtgggactctgtcccgctccaaggcacccgctctctgggacgggtgccccccggggtttttgga |
| 105863764 of | ctcctgggggtgacttagcagccgtctgcttgcagttggacttcccaggccgacagtggtctggcttct |
| chromosome 14, | gaggggtcaggccagaatgtggggtacgtgggaggccagcagagggttccatgagaagggcagg |
| GRCh38.p13 | acagggccacggacagtcagcttccatgtgacgcccggagacagaaggtctctgggtggctgggttt |
| (SEQ ID NO: 6) | ttgtggggtgaggatggacattctgccattgtgattactactactactactacatggacgtctggggcaa |
| | agggaccacggtcaccgtctcctcaggtaagaatggccactctagggcctttgttttctgctactgcctg |
| | tggggtttcctgagcattgcaggttggtcctcggggcatgttccgaggggacctgggggactggcca |
| | ggaggggatgggcactggggtgccttgaggatctgggagcctctgtggattttccgatgcctttggaa |
| | aatgggactcaggttgggtgcgtc |
| PGK-hygromycin | gggtaggggaggcgcttttcccaaggcagtctggagcatgcgctttagcagccccgctgggcacttg |
| phosphotransferase, | gcgctacacaagtggcctctggcctcgcacacattccacatccaccggtaggcgccaaccggctccg |
| hygromycin is | ttctttggtggccccttcgcgccaccttctactcctcccctagtcaggaagttcccccccgccccgcagc |
| underlined | tcgcgtcgtgcaggacgtgacaaatggaagtagcacgtctcactagtctcgtgcagatggacagcac |
| (SEQ ID NO: 7) | cgctgagcaatggaagcgggtaggcctttggggcagcggccaatagcagctttgctccttcgctttctg |
| | ggctcagaggctgggaaggggtgggtccggggggggctcaggggcgggctcagggggggg |
| | cgggcgcccgaaggtcctccggaggcccggcattctgcacgcttcaaaagcgcacgtctgccgcgc |
| | tgttctcctcttcctcatctccgggcctttcgataacttcgtataatgtatgctatacgaagttatatgaa |
| | aaagcctgaactcaccgcgacgtctgtcgagaagtttctgatcgaaaagttcgacagcgtctccgacctgat |
| | gcagctctcggagggcgaagaatctcgtgctttcagcttcgatgtaggagggcgtggatatgtcctgc |
| | gggtaaatagctgcgccgatggtttctacaaagatcgttatgtttatcggcactttgcatcggccgcgctc |
| | ccgattccggaagtgcttgacattggggaattcagcgagagcctgacctattgcatctcccgccgtgca |
| | cagggtgtcacgttgcaagacctgcctgaaaccgaactgcccgctgttctgcagccggtcgcggagg |
| | ccatggatgcgatcgctgcggccgatcttagccagacgagcgggttcggcccattcggaccgcaag |
| | gaatcgctcaatacactacatggcgtgatttcatatgcgcgattcctgatccccatgtgtatcactggcaa |
| | actctgatggacgacaccctcagtccctccgtcgcccaggctctcgatgagctgatgctttgggccga |
| | ggactgccccgaagtccggcacctcgtgcacgcggatttcggctccaacaatgtcctgacggacaat |
| | ggccgcataacagccgtcattgactggagcgagccgatcttcggggattcccaatacgagctcgcca |
| | acatcttcttctggagcccgtggttggcttctatggagcagcagacgcgctacttcgagcggaggcatc |
| | cggagcttgcaggatcgccgcggctccgggcctatatgctccgcattggtcttgaccaactctatcaga |
| | gcttggttgacggcaatttcgatgatgcagcttgggcgcagggtcgatgcgacgcaatcgtccgatcc |
| | ggagccgggactctcgggcctacacaaatgcccgcagaagcgcggccgtctggaccgatggctg |
| | tgtagaagt |
| 3′ homology arm | agttggagattttcagtttttagaataaaagtattagttgtggaatatacttcaggaccacctctgtgacag |
| (mouse), | catttatacagtatccgatgcatagggacaaagagtggagtggggcactttctttagatttgtgaggaat |
| coordinates | gttccgcactagattgtttaaaacttcatttgttggaaggagagctgtcttagtgattgagtcaagggaga |
| 113391186- | aaggcatctagcctcggtctcaaaagggtagttgctgtctagagaggtctggtggagcctgcaaaagt |
| 113391842 of | ccagctttcaaaggaacacagaagtatgtgtatggaatattagaagatgttgcttttactcttaagttggtt |
| chromosome 12, | cctaggaaaaatagttaaatactgtgactttaaaatgtgagagggttttcaagtactcatttttttaaatgt |
| GRCm39 | ccaaaattcttgtcaatcagtttgaggtcttgtttgtgtagaactgatattacttaaagtttaaccgaggaa |
| (SEQ ID NO: 8) | tgggagtgaggctctctcataacctattcagaactgacttttaacaataataaattaagtttcaaatatttt |
| | taaatgaattgagcaatgttgagttggagtcaagatggccgatcagaaccagaacacctgcagcagctggca |
| | ggaagcaggtcatctg |
| Full length (from | gccagggtctcagggtcagagtcttggaggcattttggaggtcaggaaagaaagctggggagaggg |
| homology arm to | acccttcgaatgggaacccagcctgtcctccccaagtccggccacagatgtcggcagctggggggct |
| homology arm) | ccttcggctggtctggggtgacctctctccgcttcacctggagcattctcaggggctgtcgtgatgattg |
| (SEQ ID NO: 9) | cgtggtgggactctgtcccgctccaaggcacccgctctctgggacgggtgccccccggggtttttgga |
| | ctcctgggggtgacttagcagccgtctgcttgcagttggacttcccaggccgacagtggtctggcttct |
| | gaggggtcaggccagaatgtggggtacgtgggaggccagcagagggttccatgagaagggcagg |
| | acagggccacggacagtcagcttccatgtgacgcccggagacagaaggtctctgggtggctgggttt |
| | ttgtggggtgaggatggacattctgccattgtgattactactactactactacatggacgtctggggcaa |
| | agggaccacggtcaccgtctcctcaggtaagaatggccactctagggcctttgttttctgctactgcctg |
| | tggggtttcctgagcattgcaggttggtcctcggggcatgttccgaggggacctgggcggactggcca |
| | ggaggggatgggcactggggtgccttgaggatctgggagcctctgtggattttccgatgcctttggaa |
| | aatgggactcaggttgggtgcgtcgggtaggggaggcgcttttcccaaggcagtctggagcatgcgc |
| | tttagcagccccgctgggcacttggcgctacacaagtggcctctggcctcgcacacattccacatcca |
| | ccggtaggcgccaaccggctccgttctttggtggccccttcgcgccaccttctactcctcccctagtca |
| | ggaagttcccccccgccccgcagctcgcgtcgtgcaggacgtgacaaatggaagtagcacgtctca |
| | ctagtctcgtgcagatggacagcaccgctgagcaatggaagcgggtaggcctttggggcagcggcc |
| | aatagcagctttgctccttcgctttctgggctcagaggctgggaaggggtgggtccggggggggctc |
| | aggggcgggctcaggggggggcgggcgcccgaaggtcctccggaggcccggcattctgcacgc |
| | ttcaaaagcgcacgtctgccgcgctgttctcctcttcctcatctccgggcctttcgataacttcgtataatg |
| | tatgctatacgaagttatatgaaaaagcctgaactcaccgcgacgtctgtcgagaagtttctgatcgaaa |
| | agttcgacagcgtctccgacctgatgcagctctcggagggcgaagaatctcgtgctttcagcttcgatg |
| | taggagggcgtggatatgtcctgcgggtaaatagctgcgccgatggtttctacaaagatcgttatgtttat |
| | cggcactttgcatcggccgcgctcccgattccggaagtgcttgacattggggaattcagcgagagcct |
| | gacctattgcatctcccgccgtgcacagggtgtcacgttgcaagacctgcctgaaaccgaactgcccg |
| | ctgttctgcagccggtcgcggaggccatggatgcgatcgctgcggccgatcttagccagacgagcg |
| | ggttcggcccattcggaccgcaaggaatcggtcaatacactacatggcgtgatttcatatgcgcgattg |
| | ctgatccccatgtgtatcactggcaaactgtgatggacgacaccgtcagtgcgtccgtcgcgcaggct |
| | ctcgatgagctgatgctttgggccgaggactgccccgaagtccggcacctcgtgcacgcggatttcg |
| | gctccaacaatgtcctgacggacaatggccgcataacagcggtcattgactggagcgaggcgatgtt |
| | cggggattcccaatacgaggtcgccaacatcttcttctggaggccgtggttggcttgtatggagcagca |
| | gacgcgctacttcgagcggaggcatccggagcttgcaggatcgccgcggctccgggcgtatatgctc |
| | cgcattggtcttgaccaactctatcagagcttggttgacggcaatttcgatgatgcagcttgggcgcagg |
| | gtcgatgcgacgcaatcgtccgatccggagccgggactgtcgggcgtacacaaatcgcccgcagaa |
| | gcgcggccgtctggaccgatggctgtgtagaagtcgcgtctgcgttcgaccaggctgcgcgttctcgc |
| | ggccatagcaaccgacgtacggcgttgcgccctcgccggcagcaagaagccacggaagtccgccc |
| | ggagcagaaaatgcccacgctactgcgggtttatatagacggtccccacgggatggggaaaaccac |
| | caccacgcaactgctggtggccctgggttcgcgcgacgatatcgtctacgtacccgagccgatgactt |
| | actggcgggtgctgggggcttccgagacaatcgcgaacatctacaccacacaacaccgcctcgacc |
| | agggtgagatatcggccggggacgcggcggtggtaatgacaagcgcccagataacaatgggcatg |
| | ccttatgccgtgaccgacgccgttctggctcctcatatcgggggggaggctgggagctcacatgcccc |
| | gcccccggccctcaccctcatcttcgaccgccatcccatcgccgccctcctgtgctacccggccgcgc |
| | ggtaccttatgggcagcatgaccccccaggccgtgctggcgttcgtggccctcatcccgccgaccttg |
| | cccggcaccaacatcgtgcttggggcccttccggaggacagacacatcgaccgcctggccaaacgc |
| | cagcgccccggcgagcggctggacctggctatgctggctgcgattcgccgcgtttacgggctacttg |
| | ccaatacggtgcggtatctgcagtgcggcgggtcgtgggggaggactggggacagctttcgggga |
| | cggccgtgccgccccagggtgccgagccccagagcaacggggcccacgaccccatatcgggga |
| | cacgttatttaccctgtttcgggcccccgagttgctggcccccaacggcgacctgtataacgtgtttgcc |
| | tgggccttggacgtcttggccaaacgcctccttccatgcacgtctttatcctggattacgaccaatcgc |
| | ccgccggctgccgggacgccctgctgcaacttacctccgggatggtccagacccacgtcaccaccc |
| | ccggctccataccgacgatatgcgacctggcgcgcacgtttgcccgggagatgggggaggctaact |
| | gagtcgacgactgtgccttctagttgccagccatctgttgtttgcccctcccccgtgccttccttgaccct |
| | ggaaggtgccactcccactgtcctttcctaataaaatgaggaaattgcatcgcattgtctgagtaggtgt |
| | cattctattctggggggtggggggggcaggacagcaagggggaggattgggaagacaatagcag |
| | gcatgctggggatgcggtgggctctatggagttggagattttcagtttttagaataaaagtattagttgtg |
| | gaatatacttcaggaccacctctgtgacagcatttatacagtatccgatgcatagggacaaagagtgga |
| | gtggggcactttctttagatttgtgaggaatgttccgcactagattgtttaaaacttcatttgttggaagga |
| | gagctgtcttagtgattgagtcaagggagaaaggcatctagcctcggtctcaaaagggtagttgctgtcta |
| | gagaggtctggtggagcctgcaaaagtccagctttcaaaggaacacagaagtatgtgtatggaatatta |
| | gaagatgttgcttttactcttaagttggttcctaggaaaaatagttaaatactgtgactttaaaatgtgaga |
| | gggttttcaagtactcatttttttaaatgtccaaaattcttgtcaatcagtttgaggtcttgtttgtgtaga |
| | actgatattacttaaagtttaaccgaggaatgggagtgaggctctctcataacctattcagaactgactttt |
| | aacaataataaattaagtttcaaatatttttaaatgaattgagcaatgttgagttggagtcaagatggccga |
| | tcagaaccagaacacctgcagcagctggcaggaagcaggtcatctg |

TABLE 7
sgRNA sequence

| sgRNA | sequence | SEQ ID NO |
|---|---|---|
| Mouse igh 5′ | agcctctgcacaatgctcagNGG | 10 |
| Mouse igh 3′ | tgctaaaacaatcctatggcNGG | 11 |
| Human IGH 5′ | cccagagcttgatatatagtNGG | 12 |
| Human IGH 3′ | ctcaggttgggtgcgtctgaNGG | 13 |

In Table 7, sgRNA sequences with the PAM sequence (NGG) located on the non-target strand 3′ of the sgRNA targeting sequence are provided The corresponding sgRNA targeting sequences without the PAM are provided as SEQ ID NOS: 14-17.

2.2. EHC establishment by CRE-Loxp mediated chromosome rearrangement (CMCR)

To obtain mouse EHS cells humanized for their variable domains of the Igh gene, the ˜3 MB variable domains of Igh gene on mouse chromosome 12 was replaced with ˜1 MB variable domains of IGH gene on human chromosome 14 by CRE-Loxp mediated chromosome rearrangement (CMCR; FIG. 4B). Four plasmids were designed to mediate the CMCR process.

The mouse Igh 5′ (pCM V-GFP-BGH-1 PolyA-Loxp) and 3′ (BGH poly A-Loxp-511-Hygromycin-BGH poly A-PGK-BSD-BGH PolyA) plasmids were designed to insert into 5′ and 3′ end of the mouse Igh variable loci, respectively. Simultaneously, the human 1GH 5′ (BGH polyA-Loxp-Puro-BGH PolyA-PGK-Neomycin-BGH Poly A) and 3′ (pCMV-BGP-BGH Poly A-PGK-Loxp-511) plasmids were designed to insert into 5′ and 3′ end of the human IGH variable loci, respectively (FIG. 5). The EHS cells after transfection were cultured in mouse ES cell medium containing BSD and Neomycin for 7 days. Survived GFP- and BFP-double positive cells were picked for further culturing. Genotyping was performed to identify the desired single clones with successful integration of the above plasmids. Cre was transfected into the successfully integrated EHS cells for CMCR, and the successfully rearranged cells could survive in medium containing Puromycin and Hygromycin. The survived cells were then pocked for genotyping. To facilitate the expression of human IGH gene in the EHS cells with successful CMCR, the 3′ selection marker was next deleted from the genome (FIG. 5). Following the above processes, engineered humanized chromosome (EHC; the Igh gene of mouse chromosome 12 were humanized for their variable domains) was successfully established by CMCR in EHS cells

### Example 3: Chromosomal Replacement in Mouse Embryonic Stem Cells via Micro-Cell Mediated Chromosome Transfer

Having obtained the EHS cells with an engineered humanized chromosome (EHC) as described in Examples 1 and 2, the EHC was next transferred to mouse ES cells by micro-cell mediated chromosome transfer (MMCT) to establish mouse ES cells humanized for the variable domains of Igh gene.

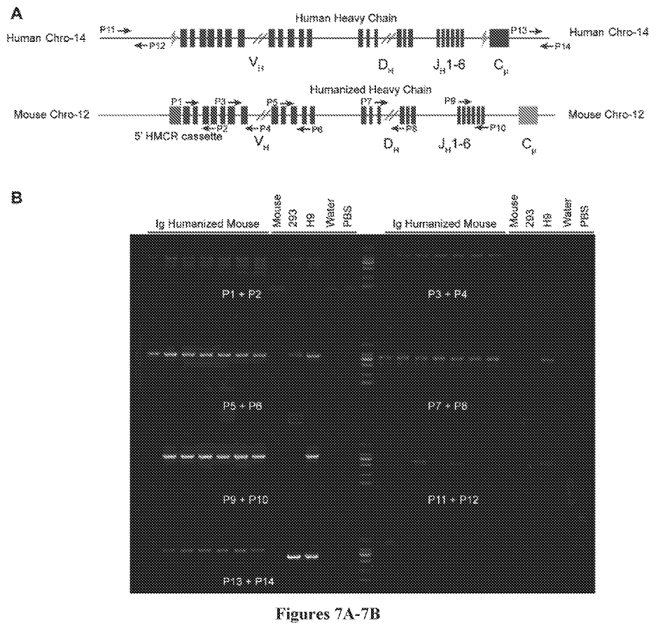

EHS cells carrying the EHC were treated with 0.2 pg/ml colcemid at 37° C. for 48 hours. Prolonged mitotic arrest induced the formation of microcells, which were collected by centrifugation (FIG. 6). Simultaneously, mouse ES cells expressing and an mCherry fluorescent marker on chromosome 12 were obtained (FIG. 6). These cells were obtained by inserting a cassette of CMV-mCherry-polyA into one copy of mouse chromosome 12.

Next, the microcells were hybridized with mouse ES cells by electrofusion, and the resulting cells were sorted by using GFP+ and mCherry+ markers by FACS to obtain mouse ES cells that were GFP-+ and mCherry-. GFP+ indicated that the EHC was successfully transferred into the mouse ES cells, while the mCherry+ marker indicated that the cells also carried the mCherry+ chromosome 12. Positive cells were continuously cultured in mouse ES cell medium for 2 weeks, and mCherry- and GFP+ mouse ES cells, i.e. cells that had lost the extra chromosome 12 marked with mCherry+, were sorted by FACS and cultured for 7 days. Single clones were isolated into separate wells for growth and karyotype analysis, and clones with the right karyotype were retained. The result was mouse ES cells humanized for their variable regions of Igh gene.

### Example 4: Production Igh Humanized Mice

The mouse ES cells humanized for their variable regions of Igh gene obtained in Example 3 were injected into blastocysts from the B6D2F1 (C57BL/6 X DBA2) mouse strain according to standard procedures. Alternatively, nuclear transfer or tetraploid embryo complementation could also have been used to generate humanized mice.

Injected blastocysts were transferred to the uteri of pseudopregnant ICR females at 2.5 days post coitus (dpc). Igh humanized mice were identified by the expression level of GFP under a fluorescence stereomicroscope, and GFP+ mice were further analyzed.

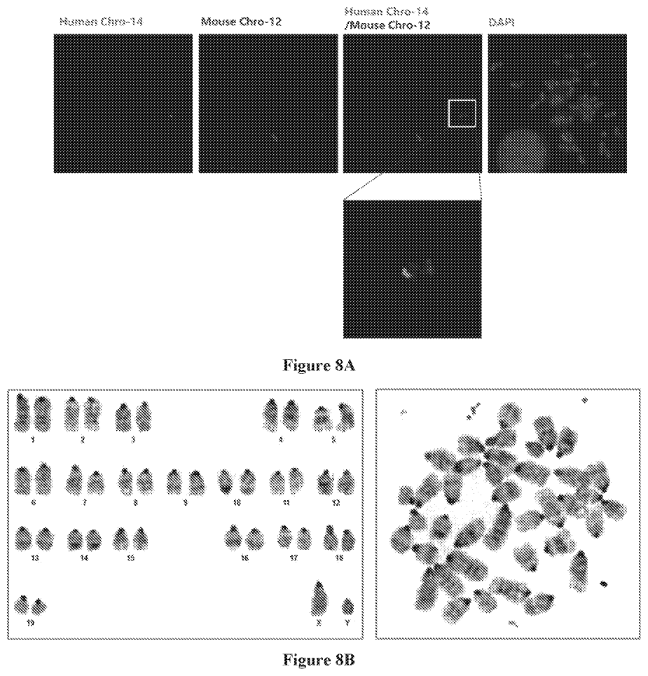

Next, a series of PCR experiments were designed to validate the Igh humanized mice. The first set of PCR experiments were designed to validate the completeness of human IGH variable regions. Five pairs of primers to different regions of human IGH variable regions were designed (see FIG. 7A, arrows that indicate PCR primers 1-10). Igh humanized mice showed positive PCR products for all the five PCR primer pairs (FIG. 7B). We also designed primers on the upstream and downstream of human IGH variable regions (FIG. 7A), and no products were observed for either of the PCR experiments for our Igh humanized mice, while the ITEK293T showed right bands of the PCR products (FIG. 7B).

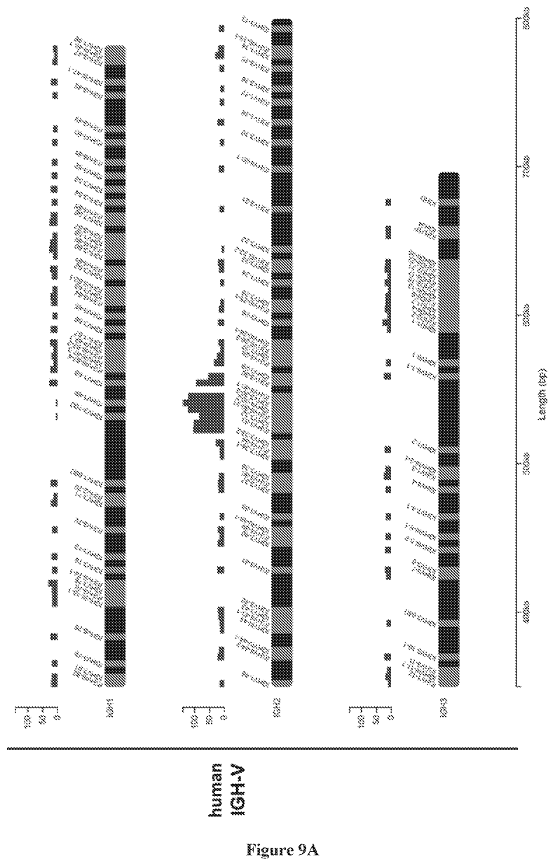

Fibroblasts were isolated from the tails of Igh humanized mice, and used to perform Fluorescence In Situ Hybridization (FISH). The FISH results showed that the chromosome 12 of Igh humanized mice contained a fragment of human chromosome 14 (FIG. 8A), indicating the variable domains of human IGH gene were successfully inserted into the chromosome 12 of mice in situ.

G-banding karyotype analysis was also performed to rule out any abnormal chromosomes (FIG. 8B).

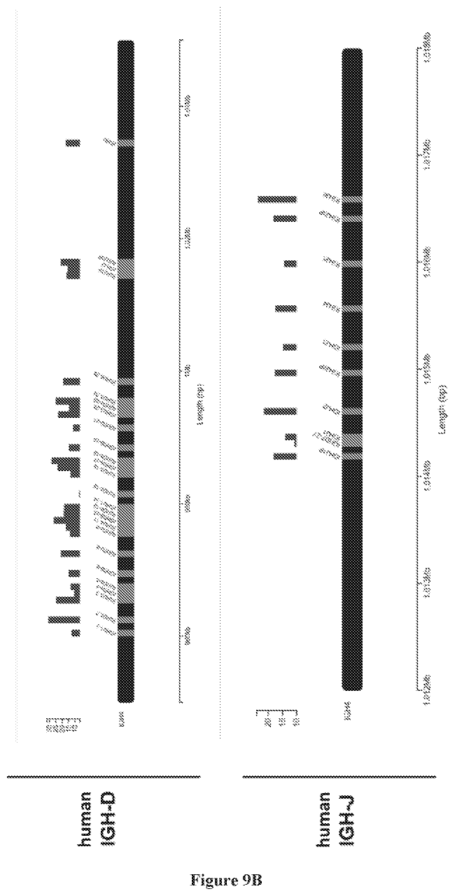

Genomic DNA of Igh humanized mice was also extracted, and whole genome sequencing (WGS) analysis was performed. WGS sequences were mapped to a reference genome containing all the chromosomes of mouse and human chromosome 14. All the variable domains of human IGH genes (VH, D_{H}, and J_{H} gene segments) were covered by the whole genome sequence reads. In addition, no off-target editing was found in other genomic regions (FIGS. 9A-9B).

### Example 5: Production Igk Humanized Mice

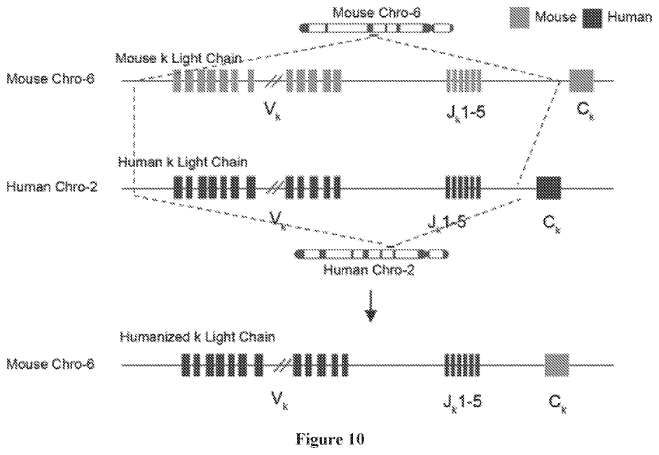

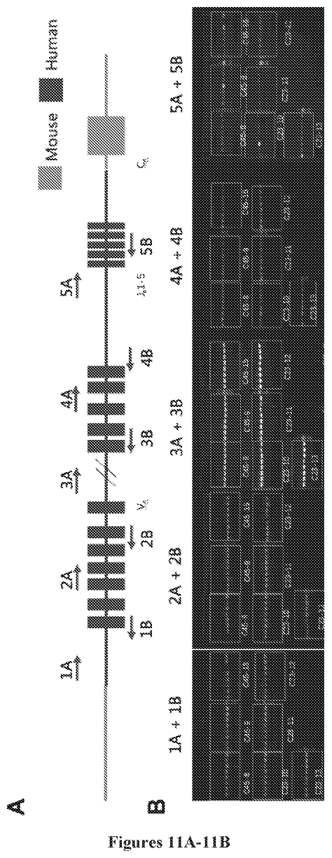

MASIRT was applied to obtain mice humanized for their variable domains of Igk gene (FIG. 10). Using similar approaches as those for Igh gene described above, we also obtained Igk humanized mice. To validate the Igk humanized mice, we firstly performed PCR experiments to validate the completeness of human IGK variable regions. Five pairs of primers on different loci of human IGK variable regions were designed (FIG. 11A), and the obtained Igk humanized mice showed positive PCR products for all the five experiments (FIG. 11B). The primers on the upstream and downstream of human IGK variable regions were also designed (FIG. 11A), and no products were observed for either of the PCR experiments for the obtained Igk humanized mice, while the HEK293T showed right bands of the PCR products (FIG. 11B). Lastly, the genomic DNA of Igk humanized mice was also extracted and performed whole genome sequencing (WGS) analysis.

TABLE 8
Exemplary 5′ plasmid sequences for HMCR mediated replacement of mouse Igk
variable region with corresponding human region

| Name | Sequence |
|---|---|
| 5′ homology arm | gggtttcccttggaattggggcttaacagcaggaactaaaaatcattggtcatcaatatctctcaacatca |
| (mouse), | atggtctcaattccccaataaaagacacaaactaacagagtggatctgtaaacagaatccatcattctgtt |
| (SEQ ID NO: 18) | gcatacaagaaacacatctcagcaaaaaagatgatcattacttcataatatagggctagaaaaaatggg |
| | cccaagaaacaagctggagtatccattctaatatcaagtgtgcatatttctaaaaggtactgctctgtaga |
| | tttggagacatattcttcagctgaagcctcagggcttgagttgcagaattgtcatctcaatttcttgagttct |
| | aatatggaacaaacactatttaaatcttcccactttgaatgagctcatggcttgctgtgtctgcctgttgact |
| | gtatctaagtggtaaaatattaactaataacgcttaattaagtaataactgaccattgggaattagatgtgtt |
| | ttttgtaacttcattgctcttttccgggcttcgttgtatcaacatttttttggtaaatggtcatcagagagtcat |
| | tccttttatagatatgcggactacttttccatttagtttccttcattggggtcccacgtctggcaaaattaaaac |
| | aaaaattcagataggattagacaggaagatgctatgctgaaaattataccctttttttgtggtgctatcttaa |
| | catgccagtgttcttgtagattgtgtctccactgatgctgacccagcctttcacagtggagtccaactgct |
| | ccctgc |
| pCMV-GFP, GFP | atagtaatcaattacggggtcattagttcatagcccatatatggagttccgcgttacataacttacggtaaa |
| is underlined | tggcccgcctggctgaccgcccaacgacccccgcccattgacgtcaataatgacgtatgttcccatag |
| | taacgccaatagggactttccattgacgtcaatgggtggagtatttacggtaaactgcccacttggcagt |
| | acatcaagtgtatcatatgccaagtccgccccctattgacgtcaatgacggtaaatggcccgcctggca |
| | ttatgcccagtacatgaccttacgggactttcctacttggcagtacatctacgtattagtcatcgctattacc |
| | atggtgatgcggttttggcagtacaccaatgggcgtggatagcggtttgactcacggggatttccaagt |
| | ctccaccccattgacgtcaatgggagtttgttttggcaccaaaatcaacgggactttccaaaatgtcgta |
| | ataaccccgccccgttgacgcaaatgggcggtaggcgtgtacggtgggaggtctatataagcagagg |
| | tcgtttagtgaaccgtcagatcAcgcgtgccaccatggtgagcaagggcgaggagctgttcaccgg |
| | ggtggtgcccatcctggtcgagctggacggcgacgtaaacggccacaagttcagcgtgtccggcga |
| | gggcgagggcgatgccacctacggcaagctgaccctgaagttcatctgcaccaccggcaagctgcc |
| | cgtgccctggcccaccctcgtgaccaccctgacctacggcgtgcagtgcttcagccgctaccccgac |
| | cacatgaagcagcacgacttcttcaagtccgccatgcccgaaggctacgtccaggagcgcaccatctt |
| | cttcaaggacgacggcaactacaagacccgcgccgagctgaagttcgagggcgacaccctggtga |
| | accgcatcgagctgaagggcatcgacttcaaggaggaccgcaacatcctggggcacaagctggagt |
| | acaactacaacagccacaacgtctatatcatggccgacaagcagaagaacggcatcaagctgaactt |
| | caagatccgccacaacatcgaggacggcagcgtgcagctcgccgaccactaccagcagaacaccc |
| | ccatcggcgacggccccgtcctgctgcccgacaaccactacctgagcacccagtccgccctgagca |
| | aagaccccaacgagaagcgcgatcacatggtcctgctggagttcgtgaccgccgccgggatcactct |
| | cggcatggacgagctgtacaagtaa |
| PGK-Puromycin N- | gggttggggttgcgccttttccaaggcagccctgggtttgcgcagggacgcggctgctctgggcgtg |
| acetyltransferase | gttccgggaaacgcagcggcgccgaccctgggactcgcacattcttcacgtccgttcgcagcgtcac |
| (SEQ ID NO: 3) | ccggatcttcgccgctacccttgtgggccccccggcgacgcttcctgctccgcccctaagtcgggaag |
| | gttccttgcggttcgcggcgtgccggacgtgacaaacggaagccgcacgtctcactagtaccctcgc |
| | agacggacagcgccagggagcaatggcagcgcgccgaccgcgatgggctgtggccaatagcggc |
| | tgctcagcagggcgcgccgagagcagcggccgggaaggggcggtgcgggaggcggggtgtgg |
| | ggcggtagtgtgggccctgttcctgcccgcgcggtgttccgcattctgcaagcctccggagcgcacgt |
| | cggcagtcggctccctcgttgaccgaatcaccgacctctctccccagggggatccaccggagcttac |
| | catgaccgagtacaagcccacggtgcgcctcgccacccgcgacgacgtccccagggccgtacgca |
| | ccctcgccgccgcgttcgccgactaccccgccacgcgccacaccgtcgatccggaccgccacatcg |
| | agcgggtcaccgagctgcaagaactcttcctcacgcgcgtcgggctcgacatcggcaaggtgtgggt |
| | cgcggacgacggcgccgcggtggcggtctggaccacgccggagagcgtcgaagcgggggcggt |
| | gttcgccgagatcggcccgcgcatggccgagttgagcggttcccggctggccgcgcagcaacagat |
| | ggaaggcctcctggcgccgcaccggcccaaggagcccgcgtggttcctggccaccgtcggcgtctc |
| | gcccgaccaccagggcaagggtctgggcagcgccgtgtgctccccggagtggaggcggccgag |
| | cgcgccggggtgcccgccttcctggaaacctccgcgccccgcaacctccccttctacgagcggctcg |
| | gcttcaccgtcaccgccgacgtcgaggtgcccgaaggaccgcgcacctggtgcatgacccgcaagc |
| | ccggtgcctgacgc |
| 3′ homology arm | ccagggatagcagggggaggaggagggaaattctgagaggtgtgccaacaggcatgaaccttgag |
| (human), | gacattgtgctagatggaataagccagccacaaaaggacaaatacattgcgatttcacttacatgaggg |
| (SEQ ID NO: 19) | gcccattatgggcaaattcaaatacagaaagagcaatggttaacaaaaggaggaagttggtgttcaat |
| | gggtatggtttccttttgcgaagatgaagaagttctggagatggacggtggtaggggatatgcgacaat |
| | gtgagtgcacttaatgccagttataacacggggagtgcgtgtgcacacggctctgggagttctcgtgca |
| | gcactcagagctcagcgtgggcgagggcgtcacccctctgggggcgtccatggggccttggagaa |
| | gggaggctccggggcaccagagcagtctaccgggagaggccgggccgagcgcttgttcaccccca |
| | gccctcttagggaactttcacatgcttctcccactaggcctaggcacccctccccaccctccctaccctc |
| | cctaccttcctggttccctgaccctcggtgactgtgtccttcaagactgaactccagagtccccacccga |
| | ggacccgcagtgcccagcccccgcgagctcacggggtgtatgcccaccccgaggctccaccgcgc |
| | ctgtgtgctgggaagcctggctccatgggaccctcgggctctgagcgcgccgtcgctgcagctgcca |
| | gcagctcctgaggaagtggctcgaggccc |
| Full length (from | gggtttcccttggaattggggcttaacagcaggaactaaaaatcattggtcatcaatatctctcaacatca |
| homology arm to | atggtctcaattccccaataaaagacacaaactaacagagtggatctgtaaacagaatccatcattctgtt |
| homology arm) | gcatacaagaaacacatctcagcaaaaaagatgatcattacttcataatatagggctagaaaaaatggg |
| (SEQ ID NO: 20) | cccaagaaacaagctggagtatccattctaatatcaagtgtgcatatttctaaaaggtactgctctgtaga |
| | tttggagacatattcttcagctgaagcctcagggcttgagttgcagaattgtcatctcaatttcttgagttct |
| | aatatggaacaaacactatttaaatcttcccactttgaatgagctcatggcttgctgtgtctgcctgttgact |
| | gtatctaagtggtaaaatattaactaataacgcttaattaagtaataactgaccattgggaattagatgtgtt |
| | ttttgtaacttcattgctcttttccgggcttcgttgtatcaacatttttttggtaaatggtcatcagagagtcat |
| | tccttttatagatatgcggactacttttccatttagtttccttcattggggtcccacgtctggcaaaattaaaac |
| | aaaaattcagataggattagacaggaagatgctatgctgaaaattataccctttttttgtggtgctatcttaa |
| | catgccagtgttcttgtagattgtgtctccactgatgctgacccagcctttcacagtggagtccaactgct |
| | ccctgcatagtaatcaattacggggtcattagttcatagcccatatatggagttccgcgttacataacttac |
| | ggtaaatggcccgcctggctgaccgcccaacgacccccgcccattgacgtcaataatgacgtatgttc |
| | ccatagtaacgccaatagggactttccattgacgtcaatgggtggagtatttacggtaaactgcccactt |
| | ggcagtacatcaagtgtatcatatgccaagtccgccccctattgacgtcaatgacggtaaatggcccgc |
| | ctggcattatgcccagtacatgaccttacgggactttcctacttggcagtacatctacgtattagtcatcgc |
| | tattaccatggtgatgcggttttggcagtacaccaatgggcgtggatagcggtttgactcacggggattt |
| | ccaagtctccaccccattgacgtcaatgggagtttgttttggcaccaaaatcaacgggactttccaaaat |
| | gtcgtaataaccccgccccgttgacgcaaatgggggtaggcgtgtacggtgggaggtctatataag |
| | cagaggtcgtttagtgaaccgtcagatcACGCGTgccaccATGGTGAGCAAGGGC |
| | GAGGAGCTGTTCACCGGGGTGGTGCCCATCCTGGTCGAGCTG |
| | GACGGCGACGTAAACGGCCACAAGTTCAGCGTGTCCGGCGA |
| | GGGCGAGGGCGATGCCACCTACGGCAAGCTGACCCTGAAGTT |
| | CATCTGCACCACCGGCAAGCTGCCCGTGCCCTGGCCCACCCT |
| | CGTGACCACCCTGACCTACGGCGTGCAGTGCTTCAGCCGCTA |
| | CCCCGACCACATGAAGCAGCACGACTTCTTCAAGTCCGCCAT |
| | GCCCGAAGGCTACGTCCAGGAGCGCACCATCTTCTTCAAGGA |
| | CGACGGCAACTACAAGACCCGCGCCGAGGTGAAGTTCGAGG |
| | GCGACACCCTGGTGAACCGCATCGAGCTGAAGGGCATCGACT |
| | TCAAGGAGGACGGCAACATCCTGGGGCACAAGCTGGAGTAC |
| | AACTACAACAGCCACAACGTCTATATCATGGCCGACAAGCAG |
| | AAGAACGGCATCAAGGTGAACTTCAAGATCCGCCACAACATC |
| | GAGGACGGCAGCGTGCAGCTCGCCGACCACTACCAGCAGAA |
| | CACCCCCATCGGCGACGGCCCCGTGCTGCTGCCCGACAACCA |
| | CTACCTGAGCACCCAGTCCGCCCTGAGCAAAGACCCCAACGA |
| | GAAGCGCGATCACATGGTCCTGCTGGAGTTCGTGACCGCCGC |
| | CGGGATCACTCTCGGCATGGACGAGCTGTACAAGTAActgtgcctt |
| | ctagttgccagccatctgttgtttgcccctcccccgtgccttccttgaccctggaaggtgccactcccact |
| | gtcctttcctaataaaatgaggaaattgcatcgcattgtctgagtaggtgtcattctattctgggggggg |
| | ggtggggcaggacagcaagggggaggattgggaagacaatagcaggcatgctggggatgcggtg |
| | ggctctatggcggcgcgccggggttggggttgcgccttttccaaggcagccctgggtttgcgcaggg |
| | acgcggctgctctgggcgtggttccgggaaacgcagcggcgccgaccctgggactcgcacattcttc |
| | acgtccgttcgcagcgtcacccggatcttcgccgctacccttgtgggccccccggcgacgcttcctgc |
| | tccgcccctaagtcgggaaggttccttgcggttcgcggcgtgccggacgtgacaaacggaagccgc |
| | acgtctcactagtaccctcgcagacggacagcgccagggagcaatggcagcgcgccgaccgcgat |
| | gggctgtggccaatagcggctgctcagcagggcgcgccgagagcagcggccgggaaggggcgg |
| | tgcgggaggcggggtgtggggcggtagtgtgggccctgttcctgcccgcgcggtgttccgcattctg |
| | caagcctccggagcgcacgtcggcagtcggctccctcgttgaccgaatcaccgacctctctccccag |
| | ggggatccaccggagcttaccatgaccgagtacaagcccacggtgcgcctcgccacccgcgacga |
| | cgtccccagggccgtacgcaccctcgccgccgcgttcgccgactaccccgccacgcgccacaccgt |
| | cgatccggaccgccacatcgagcgggtcaccgagctgcaagaactcttcctcacgcgcgtcgggct |
| | cgacatcggcaaggtgtgggtcgcggacgacggcgccgcggtggcggtctggaccacgccggag |
| | agcgtcgaagcgggggcggtgttcgccgagatcggcccgcgcatggccgagttgagcggttcccg |
| | gctggccgcgcagcaacagatggaaggcctcctggcgccgcaccggcccaaggagcccgcgtgg |
| | ttcctggccaccgtcggcgtctcgcccgaccaccagggcaagggtctgggcagcgccgtcgtgctc |
| | cccggagtggaggcggccgagcgcgccggggtgcccgccttcctggaaacctccgcgccccgca |
| | acctccccttctacgagcggctcggcttcaccgtcaccgccgacgtcgaggtgcccgaaggaccgc |
| | gcacctggtgcatgacccgcaagcccggtgcctgacgcAAGTAActgtgccttctagttgccag |
| | ccatctgttgtttgcccctcccccgtgccttccttgaccctggaaggtgccactcccactgtcctttcctaa |
| | taaaatgaggaaattgcatcgcattgtctgagtaggtgtcattctattctggggggggggtggggcag |
| | gacagcaagggggaggattgggaagacaatagcaggcatgctggggatgcgggggctctatggc |
| | ccagggatagcagggggaggaggagggaaattctgagaggtgtgccaacaggcatgaaccttgag |
| | gacattgtgctagatggaataagccagccacaaaaggacaaatacattgcgatttcacttacatgaggg |
| | gcccattatgggcaaattcaaatacagaaagagcaatggttaacaaaaggaggaagttggtgttcaat |
| | gggtatggtttccttttgcgaagatgaagaagttctggagatggacggtggtaggggatatgcgacaat |
| | gtgagtgcacttaatgccagttataacacggggagtgcgtgtgcacacggctctgggagttctcgtgca |
| | gcactcagagctcagcgtgggcgagggcgtcacccctctgggggcgtccatggggccttggagaa |
| | gggaggctccggggcaccagagcagtctaccgggagaggccgggccgagcgcttgttcaccccca |
| | gccctcttagggaactttcacatgcttctcccactaggcctaggcacccctccccaccctccctaccctc |
| | cctaccttcctggttccctgaccctcggtgactgtgtccttcaagactgaactccagagtccccacccga |
| | ggacccgcagtgcccagcccccgcgagctcacggggtgtatgcccaccccgaggctccaccgcgc |
| | ctgtgtgctgggaagcctggctccatgggaccctcgggctctgaggcgccgtcgctgcagctgcca |
| | gcagctcctgaggaagtggctcgaggccc |

TABLE 9
Exemplary 3′ plasmid sequences for HMCR mediated replacement of mouse
Igk variable region with corresponding human region

| Name | Sequence |
|---|---|
| 5′ homology arm | aaaatcagcagcaatgttgtttttagagtctgtaataagtaataaactcaaaaagacacattctataggaat |
| (human), | aagggcttcacagatagagctcattttttaaaaatccaatttgtacattagactaaacgtgaaattatctctt |
| (SEQ ID NO: 21) | attgtaatggtggaaaggtggttattcccaaaagctcaatctcaaagaaatgtgtttaaatgaaaaaaagt |
| | aaataattgcattttttaatgaccgtgggtctgtgaaaaaaataggaaatattttaaagagtatgttctttca |
| | ttatcctctgttattacttgtctacatttttattctgccaagaaggccgtggcaccgcgagctgtagacagag |
| | ccgcggtctttctcgattgagtggctttggtggccatgccaccgcgctcttggggcagccgccttgccg |
| | ctagtggccgtggccaccctgtgtctgcccgattgatgctgccgtagccagctttcctgatgcacagtg |
| | atacaaataatgccactaagggaaagagaacagaaacgtaatgggcgctgagctgggaaaaccagg |
| | gagaagactgatttattagagatttcagaaataaaattcacattcattatgatatctcattagtgaaaatttc |
| | cattaggggattgtaaataatttaaagcttttttttttttcagtgctatttaattatttcaatatcctctcat |
| | caaatgtatttaaataacaaaagctcaaccaaaaagaaagaaatatgtaattctttcagagtaaaaatcacac |
| | ccatgacctggccac |
| PGK-hygromycin | gggtaggggaggcgcttttcccaaggcagtctggagcatgcgctttagcagccccgctgggcacttg |
| phosphotransferase, | gcgctacacaagtggcctctggcctcgcacacattccacatccaccggtaggcgccaaccggctccg |
| hygromycin is | ttctttggtggccccttcgcgccaccttctactcctcccctagtcaggaagttcccccccgccccgcagc |
| underlined | tcgcgtcgtgcaggacgtgacaaatggaagtagcacgtctcactagtctcgtgcagatggacagcac |
| (SEQ ID NO: 7) | cgctgagcaatggaagcgggtaggcctttggggcagcggccaatagcagctttgctccttcgctttctg |
| | ggctcagaggctgggaaggggtgggtccggggggggctcaggggcgggctcagggggggg |
| | cgggcgcccgaaggtcctccggaggcccggcattctgcacgcttcaaaagcgcacgtctgccgcgc |
| | tgttctcctcttcctcatctccgggcctttcgataacttcgtataatgtatgctatacgaagttatatgaaaa |
| | agcctgaactcaccgcgacgtctgtcgagaagtttctgatcgaaaagttcgacagcgtctccgacctgat |
| | gcagctctcggagggcgaagaatctcgtgctttcagcttcgatgtaggagggcgtggatatgtcctgc |
| | gggtaaatagctgcgccgatgctttctacaaagatcgttatgtttatcggcactttgcatcggccgcgctc |
| | ccgattccggaagtgcttgacattggggaattcagcgagagcctgacctattgcatctcccgccgtgca |
| | cagggtctcacgttccaagacctccctgaaaccgaactgcccgctcttctgcagccggtcgcggagg |
| | ccatggatgcgatcgctgcggccgatcttagccagacgagcgggttcggcccattcggaccgcaag |
| | gaatcggtcaatacactacatggcgtgatttcatatgcgcgattgctgatccccatgtgtatcactggcaa |
| | actgtgatggacgacaccgtcagtgcgtccgtcgcgcaggctctcgatgagctgatgctttgggccga |
| | ggactgccccgaagtccggcacctcgtgcacgcggatttcggctccaacaatgtcctgacggacaat |
| | ggccgcataacagcggtcattgactggagcgaggcgatgttcggggattcccaatacgaggtcgcca |
| | acatcttcttctggaggccgtggttggcttgtatggagcagcagacgcgctacttcgagcggaggcatc |
| | cggagcttgcaggatcgccgcggctccgggcgtatatgctccgcattggtcttgaccaactctatcaga |
| | gcttgcttgacggcaatttcgatgatgcagcttgggcccagggtcgatgcgacgcaatcgtccgatcc |
| | ggagccgggactgtcgggcctacacaaatcgcccgcagaagcgcggccgtctggaccgatggctg |
| | tctagaagt |
| 3′ homology arm | tcaaggggtttttttcctttgtctcatttctacatgaaagtaaatttgaaatgatcttttttattataagagt |
| (mouse), | agaaatacagttgggtttgaactatatgttttaatggccacggttttgtaagacatttggtcctttgttttcc |
| coordinates | cagttattactcgattgtaattttatatcgccagcaatggactgaaacggtccgcaacctcttctttacaact |
| 113391186- | gggtgacctcgcggctgtgccagccatttggcgttcaccctgccgctaagggccatgtgaacccccgcggt |
| 113391842 of | agcatcccttgctccgcgtggaccactttcctgaggcacagtgataggaacagagccactaatctgaa |
| chromosome 12, | gagaacagagatgtgacagactacactaatgtgagaaaaacaaggaaagggtgacttattggagattt |
| GRCm39 | cagaaataaaatgcatttattattatattcccttattttaattttctattagggaattagaaagggcataaac |
| (SEQ ID NO: 22) | tgctttatccagtgttatattaaaagcttaatgtatataatcttttagaggtaaaatctacagccagcaaaag |
| | tcatggtaaatattctttgactgaactctcactaaactcctctaaattatatgtcatattaactggttaaatt |
| | aatataaatttgtgacatgaccttaactggttaggtaggatatttttcttcatgcaaaaatatgactaataat |
| | aatttagcacaaaaatatttcccaatactttaattctgtgatagaaaaatgtttaactcagctactataatcc |
| | c |
| Full length (from | aaaatcagcagcaatgttgtttttagagtctgtaataagtaataaactcaaaaagacacattctataggaat |
| homology arm to | aagggcttcacagatagagctcattttttaaaaatccaatttgtacattagactaaacgtgaaattatctctt |
| homology arm) | attgtaatggtggaaaggtggttattcccaaaagctcaatctcaaagaaatgtgtttaaatgaaaaaaagt |
| (SEQ ID NO: 23) | aaataattgcattttttaatgaccgtgggtctgtgaaaaaaataggaaatattttaaagagtatgttctttca |
| | ttatcctctgttattacttgtctacatttttattctgccaagaaggccgtggcaccgcgagctgtagacagag |
| | ccgcggtctttctcgattgagtggctttggtggccatgccaccgcgctcttggggcagccgccttgccg |
| | ctagtggccgtggccaccctgtgtctgcccgattgatgctgccgtagccagctttcctgatgcacagtg |
| | atacaaataatgccactaagggaaagagaacagaaacgtaatgggcgctgagctgggaaaaccagg |
| | gagaagactgatttattagagatttcagaaataaaattcacattcattatgatatctcattagtgaaaatttc |
| | cattaggggattgtaaataatttaaagcttttttttttttcagtgctatttaattatttcaatatcctctcat |
| | caaatgtatttaaataacaaaagctcaaccaaaaagaaagaaatatgtaattctttcagagtaaaaatcacac |
| | ccatgacctggccacGGGTAGGGGAGGCGCTTTTCCCAAGGCAGTCTGGA |
| | GCATGCGCTTTAGCAGCCCCGCTGGGCACTTGGCGCTACACA |
| | AGTGGCCTCTGGCCTCGCACACATTCCACATCCACCGGTAGG |
| | CGCCAACCGGCTCCGTTCTTTGGTGGCCCCTTCGCGCCACCTT |
| | CTACTCCTCCCCTAGTCAGGAAGTTCCCCCCCGCCCCGCAGCT |
| | CGCGTCGTGCAGGACGTGACAAATGGAAGTAGCACGTCTCAC |
| | TAGTCTCGTGCAGATGGACAGCACCGCTGAGCAATGGAAGCG |
| | GGTAGGCCTTTGGGGCAGCGGCCAATAGCAGCTTTGCTCCTT |
| | CGCTTTCTGGGCTCAGAGGCTGGGAAGGGGTGGGTCCGGGGG |
| | CGGGCTCAGGGGCGGGCTCAGGGGGGGGGGGGCGCCCGAA |
| | GGTCCTCCGGAGGCCCGGCATTCTGCACGCTTCAAAAGCGCA |
| | CGTCTGCCGCGCTGTTCTCCTCTTCCTCATCTCCGGGCCTTTCG |
| | ATAACTTCGTATAATGTATGCTATACGAAGTTATatgaaaaagcctga |
| | actcaccgcgacgtctgtcgagaagtttctgatcgaaaagttcgacagcgtctccgacctgatgcagct |
| | ctcggagggcgaagaatctcgtgctttcagcttcgatgtaggagggcgtggatatgtcctgcgggtaa |
| | atagctgcgccgatggtttctacaaagatcgttatgtttatcggcactttgcatcggccgcgctcccgatt |
| | ccggaagtgcttgacattggggaattcagcgagagcctgacctattgcatctcccgccgtgcacaggg |
| | tgtcacgttgcaagacctgcctgaaaccgaactgcccgctgttctgcagccggtcgcggaggccatg |
| | gatgcgatcgctgcggccgatcttagccagacgagcgggttcggcccattcggaccgcaaggaatc |
| | ggtcaatacactacatggcgtgatttcatatgcgcgattgctgatccccatgtgtatcactggcaaactgt |
| | gatggacgacaccgtcagtgcgtccgtcgcgcaggctctcgatgagctgatgctttgggccgaggac |
| | tgccccgaagtccggcacctcgtgcacgcggatttcggctccaacaatgtcctgacggacaatggcc |
| | gcataacagcggtcattgactggagcgaggcgatgttcggggattcccaatacgaggtcgccaacat |
| | cttcttctggaggccgtggttggcttgtatggagcagcagacgcgctacttcgagcggaggcatccgg |
| | agcttgcaggatcgccgcggctccgggcgtatatgctccgcattggtcttgaccaactctatcagagct |
| | tggttgacggcaatttcgatgatgcagcttgggcgcagggtcgatgcgacgcaatcgtccgatccgga |
| | gccgggactgtcgggcgtacacaaatcgcccgcagaagcgcggccgtctggaccgatggctgtgta |
| | gaagtcgcgtctgcgttcgaccaggctgcgcgttctcgcggccatagcaaccgacgtacggcgttgc |
| | gccctcgccggcagcaagaagccacggaagtccgcccggagcagaaaatgcccacgctactgcg |
| | ggtttatatagacggtccccacgggatggggaaaaccaccaccacgcaactgctggtggccctgggt |
| | tcgcgcgacgatatcgtctacgtacccgagccgatgacttactggcgggtgctgggggcttccgaga |
| | caatcgcgaacatctacaccacacaacaccgcctcgaccagggtgagatatcggccggggacgcg |
| | gcggtggtaatgacaagcgcccagataacaatgggcatgccttatgccgtgaccgacgccgttctgg |
| | ctcctcatatcgggggggaggctgggagctcacatgccccgcccccggccctcaccctcatcttcga |
| | ccgccatcccatcgccgccctcctgtgctacccggccgcgcggtaccttatgggcagcatgaccccc |
| | caggccgtgctggcgttcgtggccctcatcccgccgaccttgcccggcaccaacatcgtgcttgggg |
| | cccttccggaggacagacacatcgaccgcctggccaaacgccagcgccccggcgagcggctgga |
| | cctggctatgctggctgcgattcgccgcgtttacgggctacttgccaatacggtgcggtatctgcagtg |
| | cggcgggtcgtggcgggaggactggggacagctttcggggacggccgtgccgccccagggtgcc |
| | gagccccagagcaacgcgggcccacgaccccatatcggggacacgttatttaccctgtttcgggccc |
| | ccgagttgctggcccccaacggcgacctgtataacgtgtttgcctgggccttggacgtcttggccaaac |
| | gcctccgttccatgcacgtctttatcctggattacgaccaatcgcccgccggctgccgggacgccctgc |
| | tgcaacttacctccgggatggtccagacccacgtcaccacccccggctccataccgacgatatgcga |
| | cctggcgcgcacgtttgcccgggagatgggggaggctaacTGAGTCGACgactgtgccttct |
| | agttgccagccatctgttgtttgcccctcccccgtgccttccttgaccctggaaggtgccactcccactgt |
| | cctttcctaataaaatgaggaaattgcatcgcattgtctgagtaggtgtcattctattctggggggtgggg |
| | tggggcaggacagcaagggggaggattgggaagacaatagcaggcatgctggggatgcggtggg |
| | ctctatggtcaaggggtttttttcctttgtctcatttctacatgaaagtaaatttgaaatgatcttttttatt |
| | ataagagtagaaatacagttgggtttgaactatatgttttaatggccacggttttgtaagacatttggtcctt |
| | tgttttcccagttattactcgattgtaattttatatcgccagcaatggactgaaacggtccgcaacctcttct |
| | ttacaactgggtgacctcgcggctgtgccagccatttggcgttcaccctgccgctaagggccatgtgaacccc |
| | cgcggtagcatcccttgctccgcgtggaccactttcctgaggcacagtgataggaacagagccactaa |
| | tctgaagagaacagagatgtgacagactacactaatgtgagaaaaacaaggaaagggtgacttattgg |
| | agatttcagaaataaaatgcatttattattatattcccttattttaattttctattagggaattagaaagggc |
| | ataaactgctttatccagtgttatattaaaagcttaatgtatataatcttttagaggtaaaatctacagccag |
| | caaaagtcatggtaaatattctttgactgaactctcactaaactcctctaaattatatgtcatattaactggt |
| | taaattaatataaatttgtgacatgaccttaactggttaggtaggatatttttcttcatgcaaaaatatgact |
| | aataataatttagcacaaaaatatttcccaatactttaattctgtgatagaaaaatgtttaactcagctacta |
| | taatccc |

TABLE 10
sgRNA sequence for replacement of mouse Igk variable region with
corresponding human region

| sgRNA | sequence | SEQ ID NO |
|---|---|---|
| Mouse igk 5′ with PAM | agtctctgctgcctacagcaNGG | 24 |
| Mouse igk 3′ with PAM | agtccttgacagacagctcaNGG | 25 |
| Human IGK 5′ with PAM | gcctatgatattacccagccNGG | 26 |
| Human IGK 3′ with PAM | acccatgacctggccactgaNGG | 27 |

In Table 10, sgRNA sequences with the PAM sequence (NGG) located on the non-target strand 3′ of the sgRNA targeting sequence are provided. The corresponding sgRNA targeting sequences without the PAM are provided as SEQ ID NOS: 28-31.

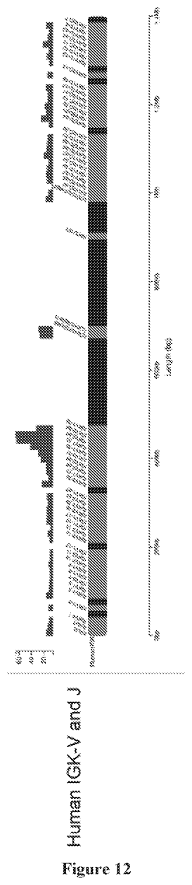

The whole genome sequences to the reference genome containing all the chromosomes of mouse and chromosome 2 of human were mapped. It shows that all the variable domains of human IGK genes (VP, and J_{H} gene segments) were covered by the whole genome sequences. Besides, no off-target edits were found in other genomic regions (FIG. 12).

1. A method of generating an engineered chromosome, comprising:
a. providing a cell comprising a target chromosome comprising a target sequence and a template chromosome comprising a template sequence;
b. contacting the cell with
i. a first nucleic acid molecule comprising from 5′ to 3′, a 5′ homology arm comprising a nucleotide sequence upstream of the 5′ end of the target sequence, at least a first marker, and a 3′ homology arm comprising a nucleotide sequence upstream of the 5′ end of the template sequence; and
ii. a second nucleic acid molecule comprising from 5′ to 3′, a 5′ homology arm comprising a nucleotide sequence downstream of the 3′ end of the template sequence, at least a second marker, and a 3′ homology arm comprising a nucleotide sequence downstream of the 3′ end of the target sequence;
c. generating a double strand break at or on both sides of the target sequence, and at the 5′ and 3′ ends of the template sequence, whereby the template sequence and the first and second markers are inserted into the target chromosome; and
d. selecting a cell or cells expressing the first and second markers.

2. The method of claim 1, wherein the first marker is located at the 5′ end of the template sequence and the second marker is located at the 3′ end of the template sequence following insertion of the template sequence.

3. The method of claim 1, wherein the 5′ and 3′ homology arms of the first and second nucleic acid molecules are between about 20 and 2,000 bp, between about 50 and 1,500 bp, between about 100 and 1,400 bp, between about 150 and 1,300 bp, between about 200 and 1,200 bp, between about 300 and 1,100 bp, between about 400 and 1,000 bp, or between about 500 and 900 bp, or between about 600 bp and 800 bp in length.

4. (canceled)

5. (canceled)

6. (canceled)

7. The method of claim 1, wherein the template sequence is between 50 KB and 250 MB, 50 KB and 100 MB, 50 KB and 50 MB, 50 KB and 20 MB, 50 KB and 10 MB, 50 KB and 5 MB, 50 KB and 3 MB, 50 KB and 2 MB, 50 KB and 1 MB, 100 KB and 200 MB, 100 KB and 100 MB, 100 KB and 50 MB, 100 KB and 20 MB, 100 KB and 10 MB, 100 KB and 5 MB, 100 KB and 3 MB, 100 KB and 2 MB, 100 KB and 1 MB, 100 KB and 500 KB, 200 KB and 100 MB, 200 KB and 50 MB, 200 KB and 20 MB, 200 KB and 10 MB, 200 KB and 5 MB, 200 KB and 3 MB, 200 KB and 2 MB, 200 KB and 1 MB, 200 KB and 500 KB, 500 KB and 100 MB, 500 KB and 50 MB, 500 KB and 20 MB, 500 KB and 10 MB, 500 KB and 5 MB, 500 KB and 3 MB, 500 KB and 2 MB, 500 KB and 1 MB, 1 MB and 100 MB, 1 MB and 50 MB, 1 MB and 20 MB, 1 MB and 10 MB, 1 MB and 5 MB, 1 MB and 3 MB, 1 MB and 2 MB, 3 MB and 100 MB, 3 MB and 50 MB, 3 MB and 20 MB, 3 MB and 10 MB, 3 MB and 5 MB, 5 MB and 100 MB, 5 MB and 50 MB, 5 MB and 20 MB, 5 MB and 10 MB, 10 MB and 100 MB, 10 MB and 50 MB, or 10 MB and 20 MB, in length.

8. (canceled)

9. The method of claim 1, wherein generating the double strand breaks at (c) comprises using a CRISPR/Cas endonuclease and one or more guide nucleic acids (gNAs), one or more zinc finger nucleases, one or more Transcription Activator-Like Effector Nucleases (TALENs), or one or more CRE recombinase to induce the double strand breaks.

10. (canceled)

11. (canceled)

12. (canceled)

13. (canceled)

14. The method of claim 1, wherein the target chromosome comprises, from 5′ to 3′, the sequence of the 5′ homology arm of the first nucleic acid molecule, the target sequence, and the sequence of 3′ homology arm of the second nucleic acid molecule.

15. The method of claim 1, wherein the template chromosome comprises, from 5′ to 3′, the sequence of the 3′ homology arm of the first nucleic acid molecule, the template sequence, and the sequence of the 5′ homology arm of the second nucleic acid molecule.

16. (canceled)

17. The method of claim 1, wherein the target sequence comprises one or more genes that are homologous to one or more genes of the template sequence.

18. The method of claim 1, wherein the template sequence comprises a naturally occurring sequence.

19. The method of claim 18, wherein the template sequence comprises one or more modifications to the naturally occurring sequence.

20. (canceled)

21. The method of claim 1, wherein the template sequence comprises an artificial sequence.

22. The method of claim 21, wherein the artificial sequence comprises a sequence encoding one or more antibodies or antigen binding fragments thereof.

23. (canceled)

24. The method of claim 1, wherein the target sequence is deleted by the insertion of the template sequence.

25. The method of claim 24, wherein:
a. the target chromosome comprises, from 5′ to 3′, the sequence of the 5′ homology arm of the first nucleic acid molecule, a first sgRNA target sequence, the target sequence, a second sgRNA target sequence, and the sequence of 3′ homology arm of the second nucleic acid molecule; and
b. the template chromosome comprises, from 5′ to 3′, a third sgRNA target sequence, the sequence of the 3′ homology arm of the first nucleic acid molecule, the template sequence, the sequence of the 5′ homology arm of the second nucleic acid molecule, and a fourth sgRNA target sequence.

26. The method of claim 25, wherein generating the double stranded breaks comprises contacting the cell with a CRISPR/Cas endonuclease, and the first, second, third, and fourth sgRNAs.

27. (canceled)

28. (canceled)

29. The method of claim 1, wherein inserting the template sequence comprises little or no deletion of a sequence of the target sequence.

30. (canceled)

31. (canceled)

32. The method of claim 29, wherein
a. the target chromosome comprises, from 5′ to 3′, the sequence of the 5′ homology arm of the first nucleic acid molecule, a first sgRNA target sequence, and the sequence of 3′ homology arm of the second nucleic acid molecule; and
b. the template chromosome comprises, from 5′ to 3′, a second sgRNA target sequence, the sequence of the 3′ homology arm of the first nucleic acid molecule, the template sequence, the sequence of the 5′ homology arm of the second nucleic acid molecule, and a third sgRNA target sequence.

33. The method of claim 32, wherein generating the double stranded breaks comprises contacting the cell with a CRISPR/Cas endonuclease, and a first, second, and third sgRNA.

34. (canceled)

35. (canceled)

36. The method of claim 1, wherein the first or second marker comprises a fluorescent protein operably linked to a promoter capable of expressing the fluorescent protein in the cell.

37. (canceled)

38. (canceled)

39. The method of claim 1, wherein the first marker further comprises a selectable marker, and/or the second marker further comprises a selectable marker and/or the second marker further comprises a selectable marker.

40. (canceled)

41. The method of claim 39, wherein the selectable marker is selected from the group consisting of Dihydrofolate reductase (DHFR), Glutamine synthase (GS), Puromycin acetyltransferase, Blasticidin deaminase, Histidinol dehydrogenase, Hygromycin phosphotransferase (hph), Bleomycin resistance gene and Aminoglycoside phosphotransferase (Neomycin resistance).

42. (canceled)

43. (canceled)

44. The method of claim 1, further comprising (e) deleting all or a part of the first or second marker after step (d).

45. (canceled)

46. The method of claim 1, wherein the cells comprise hybrid cells, embryonic hybrid stem (EHS) cells or zygotes.

47. (canceled)

48. The method of claim 46, wherein the EHS cells are generated by fusing human embryonic stem cells to embryonic stem cells from a non-human species.

49. (canceled)

50. The method of claim 46, wherein the EHS cells are generated by fusing ES cells from any two different species selected from the group consisting of mouse, rat, rabbit, guinea pig, hamster, sheep, goat, donkey, cow, horse, camel, chicken and monkey.

51. The method of claim 46, wherein generating the hybrid cells comprises:
a. generating micronucleated human cells; and
b. fusing the micronucleated human cells with a cell from a non-human species, thereby generating a hybrid cell.

52. (canceled)

53. (canceled)

54. (canceled)

55. (canceled)

56. (canceled)

57. The method of claim 1, wherein the target chromosome comprises mouse chromosome 12 and the template chromosome comprises human chromosome 14, or wherein the target chromosome comprises mouse chromosome 6 and the template chromosome comprises human chromosome 2.

58. The method of claim 57, wherein the target sequence comprises a mouse Igh variable region sequence, a mouse Igk variable region sequence, and/or a mouse Igl variable region sequence.

59. (canceled)

60. The method of claim 57, wherein the template sequence comprises a human IGH variable region sequence, a human IGK variable region sequence, and/or a human IGL variable region sequence.

61. (canceled)

62. The method of claim 1, further comprising recovering the engineered chromosome from the cells selected at step (d).

63. (canceled)

64. (canceled)

65. An engineered chromosome produced by the methods of claim 1.

66. (canceled)

67. (canceled)

68. (canceled)

69. A cell comprising the engineered chromosome of claim 65.

70. (canceled)

71. (canceled)

72. (canceled)

73. (canceled)

74. (canceled)

75. A method of generating a mouse embryonic stem cell, comprising:
a. fusing micronucleated cells comprising the engineered chromosome produced by the methods of claim 1 to mouse ES cells, wherein:
i. the mouse ES cells comprise a chromosome homologous to the engineered chromosome, the homologous chromosome comprising a first fluorescent protein operably linked to a promoter capable of expressing the fluorescent protein in the ES cells, and
ii. at least a subset of the micronucleated cells comprise the engineered chromosome, and wherein the engineered chromosome comprises a second fluorescent protein different from the first fluorescent protein, the second fluorescent protein operably linked to a promoter capable of expressing the fluorescent protein in the ES cells;
b. selecting ES cells that express both the first and second fluorescent proteins;
c. culturing the ES cells selected in step (c) until the homologous chromosome is lost by at least a subset of the ES cells; and
d. selecting ES cells that express the second fluorescent protein and do not express the first fluorescent protein.

76. (canceled)

77. (canceled)

78. (canceled)

79. A transgenic mouse, produced from the mouse ES cell produced by the method of claim 75.

80. (canceled)

81. (canceled)

82. (canceled)

83. (canceled)

84. A method of generating an antibody comprising:
a. challenging the transgenic mouse of claim 79 with an antigen, whereby the transgenic mouse generates a plurality of antibodies comprising human V, D, and J segments from the human IGH variable region; and
b. isolating an antibody specific to the antigen.

85-105. (canceled)


SEQUENCE LISTING (31 sequences)

SEQ ID NO: 1 - DNA, 800 bp, synthetic construct
  misc_feature 1..800: note = 5 homology arm (mouse)
  source 1..800: mol_type = other DNA
ctgaagtcagattgggcaacttcatagtatacaatagaaaatctacctgcagatgagttcagaaccagcagggggcacaatggggccaagaatccctagcagagagatgtggtgtgtgtgcaggggactctgcatcctctgtggtttcctttcttaacttacatgtacctgtagtgattgacatgtaacgtttccacgctcaaacactgtgaagatactttgctaaacacttcaaagatttatgttttcttgatgtgtgcatgtgtgtattcttttttgtttttagacacagggtttctctgtgtagtcctggctgccctggaactcactctgtagaccaggctggcctcgaactcagaaatctgcctgcttctgcctcccaagtgctgaagttaaagacatgtgccaccattgcctggccatgtgtgtattcttgatgcactcttctgttgacagatacacagtttatttccataatttatttattgtgatggtgctgcaataatcacttatgtacaaatgtttctgaagtatatttagttttggtcatttgggtgattatttttttctttctagtatatagcattttggaaaggtagatattaattgtatgtatgggaaggaggctgtaaattctaataacttagctgcttttgaaatttgtcctcaattctatcatccttgtaaccaccttaaatccatctattagccttgtcacaagtgagccactgtctcaggctgcaaatctttttatagattaggtcgtgatgttacatccacagcctctgcacaatgctcag

SEQ ID NO: 2 - DNA, 1312 bp, synthetic construct
  misc_feature 1..1312: note = pCMV-GFP
  source 1..1312: mol_type = other DNA
atagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcacgcgtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 3 - DNA, 1134 bp, synthetic construct
  misc_feature 1..1134: note = PGK-Puromycin N-acetyltransferase
  source 1..1134: mol_type = other DNA
gggttggggttgcgccttttccaaggcagccctgggtttgcgcagggacgcggctgctctgggcgtggttccgggaaacgcagcggcgccgaccctgggactcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgacgc

SEQ ID NO: 4 - DNA, 920 bp, synthetic construct
  misc_feature 1..920: note = 3 homology arm (human)
  source 1..920: mol_type = other DNA
agtaggagacatgcaaatagggccctcactctgctgaagaaaaccagccctgcagctctgggagaggagccccagccctgggattcccagctgtttctgcttgctgatcaggactgcacacagagaactcaccatggagtttgggctgagctgggttttccttgttgctattttaaaaggtgattcatggagaactggagatatggagtgtgaatggacatgagtgagataagcagtggatgtgtgtggcagtttctgaccagggtgtctctgtgtttgcaggtgtccagtgtgaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagagacacagtgaggggaagtcaatgtgagcccagacacaaacctcgctgcaggggcatctgagaccacgagggggtgtcctgggccctgtgaactgggctgctctccgtggcagcggctggtggtgctaaaggctgattttctctcagcatctggggctgattcatcaagtttcctcagagaacctttcagatttacaattctgtacttacgtttaatgtctctgaatgtgacactttccttccctggtgtgtctttgtttttgtgacaagaggacacattctcacctccacagaagcccgagtgtcactttggggacagaaatgaccctgccct

SEQ ID NO: 5 - DNA, 4641 bp, synthetic construct
  misc_feature 1..4641: note = Full length (from homology arm to homology arm)
  source 1..4641: mol_type = other DNA
ctgaagtcagattgggcaacttcatagtatacaatagaaaatctacctgcagatgagttcagaaccagcagggggcacaatggggccaagaatccctagcagagagatgtggtgtgtgtgcaggggactctgcatcctctgtggtttcctttcttaacttacatgtacctgtagtgattgacatgtaacgtttccacgctcaaacactgtgaagatactttgctaaacacttcaaagatttatgttttcttgatgtgtgcatgtgtgtattcttttttgtttttagacacagggtttctctgtgtagtcctggctgccctggaactcactctgtagaccaggctggcctcgaactcagaaatctgcctgcttctgcctcccaagtgctgaagttaaagacatgtgccaccattgcctggccatgtgtgtattcttgatgcactcttctgttgacagatacacagtttatttccataatttatttattgtgatggtgctgcaataatcacttatgtacaaatgtttctgaagtatatttagttttggtcatttgggtgattatttttttctttctagtatatagcattttggaaaggtagatattaattgtatgtatgggaaggaggctgtaaattctaataacttagctgcttttgaaatttgtcctcaattctatcatccttgtaaccaccttaaatccatctattagccttgtcacaagtgagccactgtctcaggctgcaaatctttttatagattaggtcgtgatgttacatccacagcctctgcacaatgctcagttaattaaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcacgcgtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggcgcgccggggttggggttgcgccttttccaaggcagccctgggtttgcgcagggacgcggctgctctgggcgtggttccgggaaacgcagcggcgccgaccctgggactcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgacgcaagtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcagtaggagacatgcaaatagggccctcactctgctgaagaaaaccagccctgcagctctgggagaggagccccagccctgggattcccagctgtttctgcttgctgatcaggactgcacacagagaactcaccatggagtttgggctgagctgggttttccttgttgctattttaaaaggtgattcatggagaactggagatatggagtgtgaatggacatgagtgagataagcagtggatgtgtgtggcagtttctgaccagggtgtctctgtgtttgcaggtgtccagtgtgaggtgcagctggtggagtccgggggaggcttagttcagcctggggggtccctgagactctcctgtgcagcctctggattcaccttcagtagctactggatgcactgggtccgccaagctccagggaaggggctggtgtgggtctcacgtattaatagtgatgggagtagcacaagctacgcggactccgtgaagggccgattcaccatctccagagacaacgccaagaacacgctgtatctgcaaatgaacagtctgagagccgaggacacggctgtgtattactgtgcaagagacacagtgaggggaagtcaatgtgagcccagacacaaacctcgctgcaggggcatctgagaccacgagggggtgtcctgggccctgtgaactgggctgctctccgtggcagcggctggtggtgctaaaggctgattttctctcagcatctggggctgattcatcaagtttcctcagagaacctttcagatttacaattctgtacttacgtttaatgtctctgaatgtgacactttccttccctggtgtgtctttgtttttgtgacaagaggacacattctcacctccacagaagcccgagtgtcactttggggacagaaatgaccctgccct

SEQ ID NO: 6 - DNA, 771 bp, synthetic construct
  misc_feature 1..771: note = 5 homology arm (human)
  source 1..771: mol_type = other DNA
gccagggtctcagggtcagagtcttggaggcattttggaggtcaggaaagaaagctggggagagggacccttcgaatgggaacccagcctgtcctccccaagtccggccacagatgtcggcagctggggggctccttcggctggtctggggtgacctctctccgcttcacctggagcattctcaggggctgtcgtgatgattgcgtggtgggactctgtcccgctccaaggcacccgctctctgggacgggtgccccccggggtttttggactcctgggggtgacttagcagccgtctgcttgcagttggacttcccaggccgacagtggtctggcttctgaggggtcaggccagaatgtggggtacgtgggaggccagcagagggttccatgagaagggcaggacagggccacggacagtcagcttccatgtgacgcccggagacagaaggtctctgggtggctgggtttttgtggggtgaggatggacattctgccattgtgattactactactactactacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgtggattttccgatgcctttggaaaatgggactcaggttgggtgcgtc

SEQ ID NO: 7 - DNA, 1505 bp, synthetic construct
  misc_feature 1..1505: note = PGK-hygromycin phosphotransferase
  source 1..1505: mol_type = other DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgataacttcgtataatgtatgctatacgaagttatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagt

SEQ ID NO: 8 - DNA, 657 bp, synthetic construct
  misc_feature 1..657: note = 3 homology arm (mouse)
  source 1..657: mol_type = other DNA
agttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggccgatcagaaccagaacacctgcagcagctggcaggaagcaggtcatctg

SEQ ID NO: 9 - DNA, 4271 bp, synthetic construct
  misc_feature 1..4271: note = Full length
  source 1..4271: mol_type = other DNA
gccagggtctcagggtcagagtcttggaggcattttggaggtcaggaaagaaagctggggagagggacccttcgaatgggaacccagcctgtcctccccaagtccggccacagatgtcggcagctggggggctccttcggctggtctggggtgacctctctccgcttcacctggagcattctcaggggctgtcgtgatgattgcgtggtgggactctgtcccgctccaaggcacccgctctctgggacgggtgccccccggggtttttggactcctgggggtgacttagcagccgtctgcttgcagttggacttcccaggccgacagtggtctggcttctgaggggtcaggccagaatgtggggtacgtgggaggccagcagagggttccatgagaagggcaggacagggccacggacagtcagcttccatgtgacgcccggagacagaaggtctctgggtggctgggtttttgtggggtgaggatggacattctgccattgtgattactactactactactacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgtggattttccgatgcctttggaaaatgggactcaggttgggtgcgtcgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgataacttcgtataatgtatgctatacgaagttatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtcgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgagtcgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggagttggagattttcagtttttagaataaaagtattagttgtggaatatacttcaggaccacctctgtgacagcatttatacagtatccgatgcatagggacaaagagtggagtggggcactttctttagatttgtgaggaatgttccgcactagattgtttaaaacttcatttgttggaaggagagctgtcttagtgattgagtcaagggagaaaggcatctagcctcggtctcaaaagggtagttgctgtctagagaggtctggtggagcctgcaaaagtccagctttcaaaggaacacagaagtatgtgtatggaatattagaagatgttgcttttactcttaagttggttcctaggaaaaatagttaaatactgtgactttaaaatgtgagagggttttcaagtactcatttttttaaatgtccaaaattcttgtcaatcagtttgaggtcttgtttgtgtagaactgatattacttaaagtttaaccgaggaatgggagtgaggctctctcataacctattcagaactgacttttaacaataataaattaagtttcaaatatttttaaatgaattgagcaatgttgagttggagtcaagatggccgatcagaaccagaacacctgcagcagctggcaggaagcaggtcatctg

SEQ ID NO: 10 - RNA, 23 nt, synthetic construct
  modified_base 5: mod_base = OTHER; note = thymine
  modified_base 7: mod_base = OTHER; note = thymine
  modified_base 14: mod_base = OTHER; note = thymine
  modified_base 17: mod_base = OTHER; note = thymine
  misc_feature 1..23: note = Mouse igh 5' sgRNA with PAM
  misc_feature 21: note = n = a, c, g or t
  source 1..23: mol_type = other RNA
agcctctgcacaatgctcagngg

SEQ ID NO: 11 - RNA, 23 nt, synthetic construct
  modified_base 1: mod_base = OTHER; note = thymine
  modified_base 4: mod_base = OTHER; note = thymine
  modified_base 12: mod_base = OTHER; note = thymine
  modified_base 15: mod_base = OTHER; note = thymine
  modified_base 17: mod_base = OTHER; note = thymine
  misc_feature 1..23: note = Mouse igh 3' sgRNA with PAM
  misc_feature 21: note = n = a, c, g or t
  source 1..23: mol_type = other RNA
tgctaaaacaatcctatggcngg

SEQ ID NO: 12 - RNA, 23 nt, synthetic construct
  modified_base 9..10: mod_base = OTHER; note = thymine
  modified_base 13: mod_base = OTHER; note = thymine
  modified_base 15: mod_base = OTHER; note = thymine
  modified_base 17: mod_base = OTHER; note = thymine
  modified_base 20: mod_base = OTHER; note = thymine
  misc_feature 1..23: note = Human IGH 5' sgRNA with PAM
  misc_feature 21: note = n = a, c, g or t
  source 1..23: mol_type = other RNA
cccagagcttgatatatagtngg

SEQ ID NO: 13 - RNA, 23 nt, synthetic construct
  modified_base 2: mod_base = OTHER; note = thymine
  modified_base 7..8: mod_base = OTHER; note = thymine
  modified_base 12: mod_base = OTHER; note = thymine
  modified_base 16: mod_base = OTHER; note = thymine
  modified_base 18: mod_base = OTHER; note = thymine
  misc_feature 1..23: note = Human IGH 3' sgRNA with PAM
  misc_feature 21: note = n = a, c, g or t
  source 1..23: mol_type = other RNA
ctcaggttgggtgcgtctgangg

SEQ ID NO: 14 - RNA, 20 nt, synthetic construct
  modified_base 5: mod_base = OTHER; note = thymine
  modified_base 7: mod_base = OTHER; note = thymine
  modified_base 14: mod_base = OTHER; note = thymine
  modified_base 17: mod_base = OTHER; note = thymine
  misc_feature 1..20: note = Mouse igh 5' sgRNA
  source 1..20: mol_type = other RNA
agcctctgcacaatgctcag

SEQ ID NO: 15 - RNA, 20 nt, synthetic construct
  modified_base 1: mod_base = OTHER; note = thymine
  modified_base 4: mod_base = OTHER; note = thymine
  modified_base 12: mod_base = OTHER; note = thymine
  modified_base 15: mod_base = OTHER; note = thymine
  modified_base 17: mod_base = OTHER; note = thymine
  misc_feature 1..20: note = Mouse igh 3' sgRNA
  source 1..20: mol_type = other RNA
tgctaaaacaatcctatggc

SEQ ID NO: 16 - RNA, 20 nt, synthetic construct
  modified_base 9..10: mod_base = OTHER; note = thymine
  modified_base 13: mod_base = OTHER; note = thymine
  modified_base 15: mod_base = OTHER; note = thymine
  modified_base 17: mod_base = OTHER; note = thymine
  modified_base 20: mod_base = OTHER; note = thymine
  misc_feature 1..20: note = Human IGH 5' sgRNA
  source 1..20: mol_type = other RNA
cccagagcttgatatatagt

SEQ ID NO: 17 - RNA, 20 nt, synthetic construct
  modified_base 2: mod_base = OTHER; note = thymine
  modified_base 7..8: mod_base = OTHER; note = thymine
  modified_base 12: mod_base = OTHER; note = thymine
  modified_base 16: mod_base = OTHER; note = thymine
  modified_base 18: mod_base = OTHER; note = thymine
  misc_feature 1..20: note = Human IGH 3' sgRNA
  source 1..20: mol_type = other RNA
ctcaggttgggtgcgtctga

SEQ ID NO: 18 - DNA, 801 bp, synthetic construct
  misc_feature 1..801: note = 5 homology arm (mouse) for Igk
  source 1..801: mol_type = other DNA
gggtttcccttggaattggggcttaacagcaggaactaaaaatcattggtcatcaatatctctcaacatcaatggtctcaattccccaataaaagacacaaactaacagagtggatctgtaaacagaatccatcattctgttgcatacaagaaacacatctcagcaaaaaagatgatcattacttcataatatagggctagaaaaaatgggcccaagaaacaagctggagtatccattctaatatcaagtgtgcatatttctaaaaggtactgctctgtagatttggagacatattcttcagctgaagcctcagggcttgagttgcagaattgtcatctcaatttcttgagttctaatatggaacaaacactatttaaatcttcccactttgaatgagctcatggcttgctgtgtctgcctgttgactgtatctaagtggtaaaatattaactaataacgcttaattaagtaataactgaccattgggaattagatgtgttttttgtaacttcattgctcttttccgggcttcgttgtatcaacatttttttggtaaatggtcatcagagagtcattccttttatagatatgcggactacttttccatttagtttccttcattggggtcccacgtctggcaaaattaaaacaaaaattcagataggattagacaggaagatgctatgctgaaaattataccctttttttgtggtgctatcttaacatgccagtgttcttgtagattgtgtctccactgatgctgacccagcctttcacagtggagtccaactgctccctgc

SEQ ID NO: 19 - DNA, 771 bp, synthetic construct
  misc_feature 1..771: note = 3 homology arm (human) for Igk
  source 1..771: mol_type = other DNA
ccagggatagcagggggaggaggagggaaattctgagaggtgtgccaacaggcatgaaccttgaggacattgtgctagatggaataagccagccacaaaaggacaaatacattgcgatttcacttacatgaggggcccattatgggcaaattcaaatacagaaagagcaatggttaacaaaaggaggaagttggtgttcaatgggtatggtttccttttgcgaagatgaagaagttctggagatggacggtggtaggggatatgcgacaatgtgagtgcacttaatgccagttataacacggggagtgcgtgtgcacacggctctgggagttctcgtgcagcactcagagctcagcgtgggcgagggcgtcacccctctgggggcgtccatggggccttggagaagggaggctccggggcaccagagcagtctaccgggagaggccgggccgagcgcttgttcacccccagccctcttagggaactttcacatgcttctcccactaggcctaggcacccctccccaccctccctaccctccctaccttcctggttccctgaccctcggtgactgtgtccttcaagactgaactccagagtccccacccgaggacccgcagtgcccagcccccgcgagctcacggggtgtatgcccaccccgaggctccaccgcgcctgtgtgctgggaagcctggctccatgggaccctcgggctctgagcgcgccgtcgctgcagctgccagcagctcctgaggaagtggctcgaggccc

SEQ ID NO: 20 - DNA, 4485 bp, synthetic construct
  misc_feature 1..4485: note = Full length (from homology arm to homology arm) for Igk
  source 1..4485: mol_type = other DNA
gggtttcccttggaattggggcttaacagcaggaactaaaaatcattggtcatcaatatctctcaacatcaatggtctcaattccccaataaaagacacaaactaacagagtggatctgtaaacagaatccatcattctgttgcatacaagaaacacatctcagcaaaaaagatgatcattacttcataatatagggctagaaaaaatgggcccaagaaacaagctggagtatccattctaatatcaagtgtgcatatttctaaaaggtactgctctgtagatttggagacatattcttcagctgaagcctcagggcttgagttgcagaattgtcatctcaatttcttgagttctaatatggaacaaacactatttaaatcttcccactttgaatgagctcatggcttgctgtgtctgcctgttgactgtatctaagtggtaaaatattaactaataacgcttaattaagtaataactgaccattgggaattagatgtgttttttgtaacttcattgctcttttccgggcttcgttgtatcaacatttttttggtaaatggtcatcagagagtcattccttttatagatatgcggactacttttccatttagtttccttcattggggtcccacgtctggcaaaattaaaacaaaaattcagataggattagacaggaagatgctatgctgaaaattataccctttttttgtggtgctatcttaacatgccagtgttcttgtagattgtgtctccactgatgctgacccagcctttcacagtggagtccaactgctccctgcatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagaggtcgtttagtgaaccgtcagatcacgcgtgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcggcgcgccggggttggggttgcgccttttccaaggcagccctgggtttgcgcagggacgcggctgctctgggcgtggttccgggaaacgcagcggcgccgaccctgggactcgcacattcttcacgtccgttcgcagcgtcacccggatcttcgccgctacccttgtgggccccccggcgacgcttcctgctccgcccctaagtcgggaaggttccttgcggttcgcggcgtgccggacgtgacaaacggaagccgcacgtctcactagtaccctcgcagacggacagcgccagggagcaatggcagcgcgccgaccgcgatgggctgtggccaatagcggctgctcagcagggcgcgccgagagcagcggccgggaaggggcggtgcgggaggcggggtgtggggcggtagtgtgggccctgttcctgcccgcgcggtgttccgcattctgcaagcctccggagcgcacgtcggcagtcggctccctcgttgaccgaatcaccgacctctctccccagggggatccaccggagcttaccatgaccgagtacaagcccacggtgcgcctcgccacccgcgacgacgtccccagggccgtacgcaccctcgccgccgcgttcgccgactaccccgccacgcgccacaccgtcgatccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggaaacctccgcgccccgcaacctccccttctacgagcggctcggcttcaccgtcaccgccgacgtcgaggtgcccgaaggaccgcgcacctggtgcatgacccgcaagcccggtgcctgacgcaagtaactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcccagggatagcagggggaggaggagggaaattctgagaggtgtgccaacaggcatgaaccttgaggacattgtgctagatggaataagccagccacaaaaggacaaatacattgcgatttcacttacatgaggggcccattatgggcaaattcaaatacagaaagagcaatggttaacaaaaggaggaagttggtgttcaatgggtatggtttccttttgcgaagatgaagaagttctggagatggacggtggtaggggatatgcgacaatgtgagtgcacttaatgccagttataacacggggagtgcgtgtgcacacggctctgggagttctcgtgcagcactcagagctcagcgtgggcgagggcgtcacccctctgggggcgtccatggggccttggagaagggaggctccggggcaccagagcagtctaccgggagaggccgggccgagcgcttgttcacccccagccctcttagggaactttcacatgcttctcccactaggcctaggcacccctccccaccctccctaccctccctaccttcctggttccctgaccctcggtgactgtgtccttcaagactgaactccagagtccccacccgaggacccgcagtgcccagcccccgcgagctcacggggtgtatgcccaccccgaggctccaccgcgcctgtgtgctgggaagcctggctccatgggaccctcgggctctgagcgcgccgtcgctgcagctgccagcagctcctgaggaagtggctcgaggccc

SEQ ID NO: 21 - DNA, 800 bp, synthetic construct
  misc_feature 1..800: note = 5 homology arm (human) for Igk
  source 1..800: mol_type = other DNA
aaaatcagcagcaatgttgtttttagagtctgtaataagtaataaactcaaaaagacacattctataggaataagggcttcacagatagagctcattttttaaaaatccaatttgtacattagactaaacgtgaaattatctcttattgtaatggtggaaaggtggttattcccaaaagctcaatctcaaagaaatgtgtttaaatgaaaaaaagtaaataattgcattttttaatgaccgtgggtctgtgaaaaaaataggaaatattttaaagagtatgttctttcattatcctctgttattacttgtctacatttttattctgccaagaaggccgtggcaccgcgagctgtagacagagccgcggtctttctcgattgagtggctttggtggccatgccaccgcgctcttggggcagccgccttgccgctagtggccgtggccaccctgtgtctgcccgattgatgctgccgtagccagctttcctgatgcacagtgatacaaataatgccactaagggaaagagaacagaaacgtaatgggcgctgagctgggaaaaccagggagaagactgatttattagagatttcagaaataaaattcacattcattatgatatctcattagtgaaaatttccattaggggattgtaaataatttaaagcttttttttttttcagtgctatttaattatttcaatatcctctcatcaaatgtatttaaataacaaaagctcaaccaaaaagaaagaaatatgtaattctttcagagtaaaaatcacacccatgacctggccac

SEQ ID NO: 22 - DNA, 792 bp, synthetic construct
  misc_feature 1..792: note = 3 homology arm (mouse) for Igk
  source 1..792: mol_type = other DNA
tcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccgcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactataatccc

SEQ ID NO: 23 - DNA, 4435 bp, synthetic construct
  misc_feature 1..4435: note = Full length for Igk
  source 1..4435: mol_type = other DNA
aaaatcagcagcaatgttgtttttagagtctgtaataagtaataaactcaaaaagacacattctataggaataagggcttcacagatagagctcattttttaaaaatccaatttgtacattagactaaacgtgaaattatctcttattgtaatggtggaaaggtggttattcccaaaagctcaatctcaaagaaatgtgtttaaatgaaaaaaagtaaataattgcattttttaatgaccgtgggtctgtgaaaaaaataggaaatattttaaagagtatgttctttcattatcctctgttattacttgtctacatttttattctgccaagaaggccgtggcaccgcgagctgtagacagagccgcggtctttctcgattgagtggctttggtggccatgccaccgcgctcttggggcagccgccttgccgctagtggccgtggccaccctgtgtctgcccgattgatgctgccgtagccagctttcctgatgcacagtgatacaaataatgccactaagggaaagagaacagaaacgtaatgggcgctgagctgggaaaaccagggagaagactgatttattagagatttcagaaataaaattcacattcattatgatatctcattagtgaaaatttccattaggggattgtaaataatttaaagcttttttttttttcagtgctatttaattatttcaatatcctctcatcaaatgtatttaaataacaaaagctcaaccaaaaagaaagaaatatgtaattctttcagagtaaaaatcacacccatgacctggccacgggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcgataacttcgtataatgtatgctatacgaagttatatgaaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggccatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtcgcgtctgcgttcgaccaggctgcgcgttctcgcggccatagcaaccgacgtacggcgttgcgccctcgccggcagcaagaagccacggaagtccgcccggagcagaaaatgcccacgctactgcgggtttatatagacggtccccacgggatggggaaaaccaccaccacgcaactgctggtggccctgggttcgcgcgacgatatcgtctacgtacccgagccgatgacttactggcgggtgctgggggcttccgagacaatcgcgaacatctacaccacacaacaccgcctcgaccagggtgagatatcggccggggacgcggcggtggtaatgacaagcgcccagataacaatgggcatgccttatgccgtgaccgacgccgttctggctcctcatatcgggggggaggctgggagctcacatgccccgcccccggccctcaccctcatcttcgaccgccatcccatcgccgccctcctgtgctacccggccgcgcggtaccttatgggcagcatgaccccccaggccgtgctggcgttcgtggccctcatcccgccgaccttgcccggcaccaacatcgtgcttggggcccttccggaggacagacacatcgaccgcctggccaaacgccagcgccccggcgagcggctggacctggctatgctggctgcgattcgccgcgtttacgggctacttgccaatacggtgcggtatctgcagtgcggcgggtcgtggcgggaggactggggacagctttcggggacggccgtgccgccccagggtgccgagccccagagcaacgcgggcccacgaccccatatcggggacacgttatttaccctgtttcgggcccccgagttgctggcccccaacggcgacctgtataacgtgtttgcctgggccttggacgtcttggccaaacgcctccgttccatgcacgtctttatcctggattacgaccaatcgcccgccggctgccgggacgccctgctgcaacttacctccgggatggtccagacccacgtcaccacccccggctccataccgacgatatgcgacctggcgcgcacgtttgcccgggagatgggggaggctaactgagtcgacgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtcaaggggtttttttcctttgtctcatttctacatgaaagtaaatttgaaatgatcttttttattataagagtagaaatacagttgggtttgaactatatgttttaatggccacggttttgtaagacatttggtcctttgttttcccagttattactcgattgtaattttatatcgccagcaatggactgaaacggtccgcaacctcttctttacaactgggtgacctcgcggctgtgccagccatttggcgttcaccctgccgctaagggccatgtgaacccccgcggtagcatcccttgctccgcgtggaccactttcctgaggcacagtgataggaacagagccactaatctgaagagaacagagatgtgacagactacactaatgtgagaaaaacaaggaaagggtgacttattggagatttcagaaataaaatgcatttattattatattcccttattttaattttctattagggaattagaaagggcataaactgctttatccagtgttatattaaaagcttaatgtatataatcttttagaggtaaaatctacagccagcaaaagtcatggtaaatattctttgactgaactctcactaaactcctctaaattatatgtcatattaactggttaaattaatataaatttgtgacatgaccttaactggttaggtaggatatttttcttcatgcaaaaatatgactaataataatttagcacaaaaatatttcccaatactttaattctgtgatagaaaaatgtttaactcagctactataatccc

SEQ ID NO: 24 - RNA, 23 nt, synthetic construct
  modified_base 3: mod_base = OTHER; note = thymine
  modified_base 5: mod_base = OTHER; note = thymine
  modified_base 7: mod_base = OTHER; note = thymine
  modified_base 10: mod_base = OTHER; note = thymine
  modified_base 14: mod_base = OTHER; note = thymine
  misc_feature 1..23: note = Mouse igk 5' with PAM
  misc_feature 21: note = n = a, c, g or t
  source 1..23: mol_type = other RNA
agtctctgctgcctacagcangg

SEQ ID NO: 25 - RNA, 23 nt, synthetic construct
  modified_base 3: mod_base = OTHER; note = thymine
  modified_base 6..7: mod_base = OTHER; note = thymine
  modified_base 18: mod_base = OTHER; note = thymine
  misc_feature 1..23: note = Mouse igk 3' with PAM
  misc_feature 21: note = n = a, c, g or t
  source 1..23: mol_type = other RNA
agtccttgacagacagctcangg

SEQ ID NO: 26 - RNA, 23 nt, synthetic construct
  modified_base 4: mod_base = OTHER; note = thymine
  modified_base 6: mod_base = OTHER; note = thymine
  modified_base 9: mod_base = OTHER; note = thymine
  modified_base 11..12: mod_base = OTHER; note = thymine
  misc_feature 1..23: note = Human IGK 5' with PAM
  misc_feature 21: note = n = a, c, g or t
  source 1..23: mol_type = other RNA
gcctatgatattacccagccngg

SEQ ID NO: 27 - RNA, 23 nt, synthetic construct
  modified_base 6: mod_base = OTHER; note = thymine
  modified_base 11: mod_base = OTHER; note = thymine
  modified_base 18: mod_base = OTHER; note = thymine
  misc_feature 1..23: note = Human IGK 3' with PAM
  misc_feature 21: note = n = a, c, g or t
  source 1..23: mol_type = other RNA
acccatgacctggccactgangg

SEQ ID NO: 28 - RNA, 20 nt, synthetic construct
  modified_base 3: mod_base = OTHER; note = thymine
  modified_base 5: mod_base = OTHER; note = thymine
  modified_base 7: mod_base = OTHER; note = thymine
  modified_base 10: mod_base = OTHER; note = thymine
  modified_base 14: mod_base = OTHER; note = thymine
  misc_feature 1..20: note = Mouse igk 5'
  source 1..20: mol_type = other RNA
agtctctgctgcctacagca

SEQ ID NO: 29 - RNA, 20 nt, synthetic construct
  modified_base 3: mod_base = OTHER; note = thymine
  modified_base 6..7: mod_base = OTHER; note = thymine
  modified_base 18: mod_base = OTHER; note = thymine
  misc_feature 1..20: note = Mouse igk 3'
  source 1..20: mol_type = other RNA
agtccttgacagacagctca

SEQ ID NO: 30 - RNA, 20 nt, synthetic construct
  modified_base 4: mod_base = OTHER; note = thymine
  modified_base 6: mod_base = OTHER; note = thymine
  modified_base 9: mod_base = OTHER; note = thymine
  modified_base 11..12: mod_base = OTHER; note = thymine
  misc_feature 1..20: note = Human IGK 5'
  source 1..20: mol_type = other RNA
gcctatgatattacccagcc

SEQ ID NO: 31 - RNA, 20 nt, synthetic construct
  misc_feature 1..20: note = Human IGK 3'
  source 1..20: mol_type = other RNA
  modified_base 6: mod_base = OTHER; note = thymine
  modified_base 11: mod_base = OTHER; note = thymine
  modified_base 18: mod_base = OTHER; note = thymine
acccatgacctggccactga